(19) United States
(12) Patent Application Publication
(10) Pub. No.: US 2025/0026741 A1
(43) Pub. Date: Jan. 23, 2025

(54) INHIBITORS OF THE ONCOGENIC SHP2 PHOSPHATASE AND USES THEREOF

(71) Applicant: Sanford Burnham Prebys Medical Discovery Institute, La Jolla, CA (US)

(72) Inventors: Nicholas David Peter COSFORD, San Diego, CA (US); Lutz TAUTZ, San Diego, CA (US); Dhanya RAVEENDRA-PANICKAR, San Diego, CA (US); Darren FINLAY, San Diego, CA (US); Lester LAMBERT, San Marcos, CA (US)

(21) Appl. No.: 18/710,429

(22) PCT Filed: Nov. 18, 2022
(86) PCT No.: PCT/US2022/050424

§ 371 Date: May 15, 2024

Related U.S. Application Data
(60) Provisional application No. 63/281,258, filed on Nov. 19, 2021.

(51) Int. Cl. C07D 409/14; A61K 31/194; A61K 31/341; A61K 31/343; A61K 31/381; A61K 31/427; A61K 31/4535; A61K 31/4709; A61K 31/496; C07C 65/40; C07D 307/54; C07D 307/56; C07D 333/64; C07D 409/06; C07D 417/06

(52) U.S. Cl. CPC C07D 409/14; A61K 31/194; A61K 31/341; A61K 31/343; A61K 31/381; A61K 31/427; A61K 31/4535; A61K 31/4709; A61K 31/496; C07C 65/40; C07D 307/54; C07D 307/56; C07D 333/64; C07D 409/06; C07D 417/06

(57) ABSTRACT

Described herein are compounds which are inhibitors of oncogenic and wild-type SHP2 and methods of treatment using SHP2 inhibitors.

### CROSS-REFERENCE

This application claims the benefit of U.S. Provisional Patent Application Ser. No. 63/281,258, filed Nov. 19, 2021; which is incorporated herein by reference in its entirety.

### STATEMENT AS TO FEDERALLY SPONSORED RESEARCH OR DEVELOPMENT

This invention was made with government support under R21 NS067502, HHSN261200800001E, and R21 CA195422 awarded by the National Institutes of Health. The government has certain rights in the invention.

### BACKGROUND

Protein tyrosine phosphorylation is a key mechanism for signal transduction. Disturbance of the dynamic balance between tyrosine phosphorylation and dephosphorylation of signaling molecules, controlled by protein tyrosine kinases (PTKs) and protein tyrosine phosphatases (PTPs), is known to be crucial for the development of cancer. In fact, most approved targeted cancer therapies are tyrosine kinase inhibitors (TKIs). PTPs on the other hand have long been stigmatized as undruggable. One PTP target is the Src-homology 2 domain-containing phosphatase 2 (SHP2), which is implicated in tumor initiation, progression, metastasis, and treatment resistance, primarily because of its role as a signaling nexus of the extracellular-signal-regulated kinase (ERK) pathway, acting upstream of Ras.

SHP2 may be an attractive therapeutic target for cancers with upregulated RTK and Ras signaling. Several compounds that allosterically inhibit wild-type SHP2 are in Phase I/II clinical trials, however, because of the unique mechanism of action, known SHP2 inhibitors are likely ineffective for patients with cancers that are driven by oncogenic mutant forms of SHP2, including pediatric and acute leukemias. Among all blood cancers, the highest rates of SHP2 gain-of-function mutations occur in juvenile myelomonocytic leukemia (JMML), with leukemogenesis driven by SHP2 variants in up to 42% of these children (source: COSMIC database, March 2021). Acute leukemias with larger patient populations such as AML and B cell acute lymphoblastic leukemia (B-ALL) have SHP2 mutation rates between 5 and 10%, and those mutations are associated with poor clinical outcomes as well as resistance to targeted therapies. Cancers driven by aberrant FGF signaling, including but not limited to many breast cancers, may be inherently resistant to known SHP2 allosteric inhibitors. The reason for this resistance may be a rapid feedback activation of the FGF receptor that leads to increased recruitment and activation of SHP2.

### BRIEF SUMMARY

Recognized herein is an urgent need for novel SHP2 inhibitory compounds that may act through different mechanisms and exhibit a new inhibition paradigm. Provided in the present disclosure are compounds, pharmaceutical composition and method of modulating the activity of SHP2 phosphatase. Also described herein are inhibitors of SHP2 phosphatase, methods of making such compounds, pharmaceutical compositions and medicaments comprising such compounds, and methods of using such compounds in the treatment of conditions, diseases, or disorders associated with SHP2 phosphatase activity.

In one aspect, described herein are compounds of Formula (I):

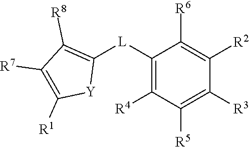

or a pharmaceutically acceptable salt thereof, wherein,
R¹ is:

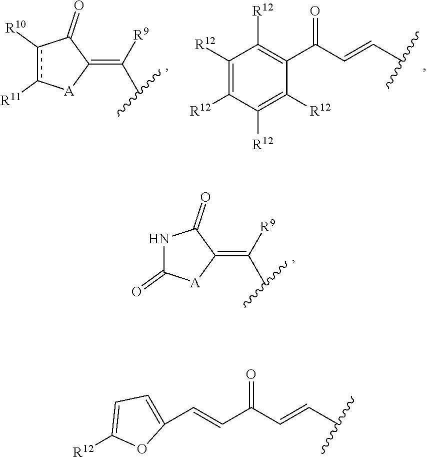

R² is —H halogen, —OH, —C(O)N(R¹²)₂, —CN, —COOH, —C(O)NR¹²N(R¹²)₂, —C(O)R¹²,

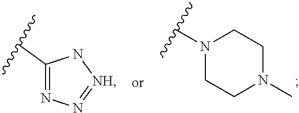

A is —CH₂—, —S—, —O—, —CH₂—CH₂—, —C(R¹²)₂—, or —C(R¹²)₂—C(R¹²)₂—;
Y is —O—, —S—, —NH— or —CH═CH—;
L is a bond, —CH═, or —C(═O)CH═CH—.
R³ is hydrogen, —OH, —F, —Cl, —CF₃, —OCF₃, or —Br;
R⁴, R⁵, R⁶, R⁷, R⁸, and R⁹ are each independently hydrogen, halogen, —OR¹³, —SR¹³, —N(R¹³)₂, —NO₂, —C(O)OR¹³, —C(O)N(R¹³)₂, —C(S)OR¹³, —C(S)SR¹³, —C(O)SR¹³, C₁-C₁₆ substituted or unsubstituted alkyl, C₁-C₁₆ substituted or unsubstituted alkenyl, substituted or unsubstituted C₆ aryl, substituted or unsubstituted C₃-C₈ cycloalkyl, substituted or unsubstituted C₃-C₈ cycloalkenyl, substituted or unsubstituted C₃-C₈ cycloalkynyl, substituted or unsubstituted heterocycloalkyl, substituted or unsubstituted heterocycloalkenyl, substituted or unsubstituted heterocycloalkynyl, or C₁-C₁₆ substituted or unsubstituted alkynyl;
R¹⁰ and R¹¹ either together form a substituted or unsubstituted 6 membered aryl ring, or are each independently hydrogen, —OR¹³, —SR¹³, —N(R¹³)₂, —NO₂, —CN, —C(O)OR¹³, —C(O)N(R¹³)₂, —C(S)OR¹³, —C(S)SR¹³, —C(O)SR¹³, C₁-C₁₆ substituted or unsubstituted alkyl, C₁-C₁₆ substituted or unsubstituted alkenyl, substituted or unsubstituted C₆ aryl, substituted or unsubstituted C₃-C₈ cycloalkyl, substituted or unsubstituted C₃-C₈ cycloalkenyl, substituted or unsubstituted C₃-C₈ cycloalkynyl, substituted or unsubstituted heterocycloalkyl, substituted or unsubstituted heterocycloalkenyl, substituted or unsubstituted heterocycloalkynyl, or C₁-C₁₆ substituted or unsubstituted alkynyl;
R¹² are each independently hydrogen, halogens, —OH, —CN, —SH, —NO₂,

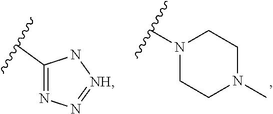

C₁-C₁₆ substituted or unsubstituted alkyl, C₁-C₁₆ substituted or unsubstituted alkyl, C₁-C₁₆ substituted or unsubstituted alkenyl, substituted or unsubstituted C₆ aryl, substituted or unsubstituted C₃-C₈ cycloalkyl, substituted or unsubstituted C₃-C₈ cycloalkenyl, substituted or unsubstituted C₃-C₈ cycloalkynyl, substituted or unsubstituted heterocycloalkyl, substituted or unsubstituted heterocycloalkenyl, substituted or unsubstituted heterocycloalkynyl, or C₁-C₁₆ substituted or unsubstituted alkynyl;
R¹³ are each independently C1-C₁₆ substituted or unsubstituted alkyl, C₁-C₁₆ substituted or unsubstituted alkyl, C₁-C₁₆ substituted or unsubstituted alkenyl, substituted or unsubstituted C₆ aryl, substituted or unsubstituted C₃-C₈ cycloalkyl, substituted or unsubstituted C₃-C₈ cycloalkenyl, substituted or unsubstituted C₃-C₈ cycloalkynyl, substituted or unsubstituted heterocycloalkyl, substituted or unsubstituted heterocycloalkenyl, substituted or unsubstituted heterocycloalkynyl, or C₁-C₁₆ substituted or unsubstituted alkynyl;
wherein
when R¹ is hydrogen, R² is

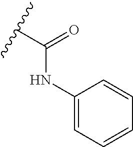 [SMILES: *C(=O)Nc1ccccc1]

### and R³ is —OH;

when A is —C(R¹²)₂—C(R¹²)₂—, R² is —C(O)OH and R³ is —OH or R³ is —C(O)OH and R² is —OH; with the proviso that the compound is not a molecule from Table 1.

In some embodiments, the compound may be a compound of Formula I where R¹ is

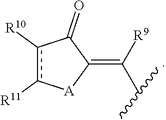

In some embodiments, R⁹ may be hydrogen. In some embodiments, A is —O—, —S—, —CH₂— or —CH₂CH₂—. In some embodiments of a compound of Formula I, R¹ is

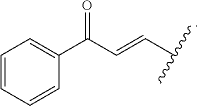 [SMILES: */C=C/C(=O)c1ccccc1]

In some embodiments, R³ is —OH or a halogen. In some embodiments of, R² is —C(═O)—N(R¹³)₂, or —C(═O)—OR¹³. In some embodiments of, R¹ is H. In some embodiments, R³ is —OH or a halogen. In some embodiments, R² is —C(═O)N(R¹³)₂. In some embodiments, R³ is —OH. In some embodiments, R² is —C═O—NHR¹³. In some embodiments, R² is

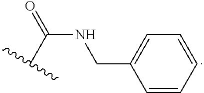 [SMILES: *C(=O)NCc1ccccc1]

In some embodiments, R¹⁰ and R¹¹ may together form a substituted or unsubstituted 6 membered aryl ring. In some embodiments, R¹⁰ and R¹¹ may each be independently selected from the group of hydrogen, —OR¹³, —SR¹³, —N(R¹³)₂, —NO₂, —CN, —C(═O)OR¹³, —C(═O)N(R¹³)₂, —C(═S)OR¹³, —C(═S)SR¹³, —C(═O)SR¹³, C₁-C₁₆ substituted or unsubstituted alkyl, C₁-C₁₆ substituted or unsubstituted alkenyl, substituted or unsubstituted C₆ aryl, substituted or unsubstituted C₃-C₈ cycloalkyl, substituted or unsubstituted C₃-C₈ cycloalkenyl, substituted or unsubstituted C₃-C₈ cycloalkynyl, substituted or unsubstituted heterocycloalkyl, substituted or unsubstituted heterocycloalkenyl, substituted or unsubstituted heterocycloalkynyl, or C₁-C₁₆ substituted or unsubstituted alkynyl.

In some embodiments, R⁹ is hydrogen. In some embodiments, R⁹ is hydrogen and R³ is —OH or a halogen. In some embodiments, R² is —C(═O)—N(R¹³)₂, or —C(═O)—OR¹³. In some embodiments, the compound is:

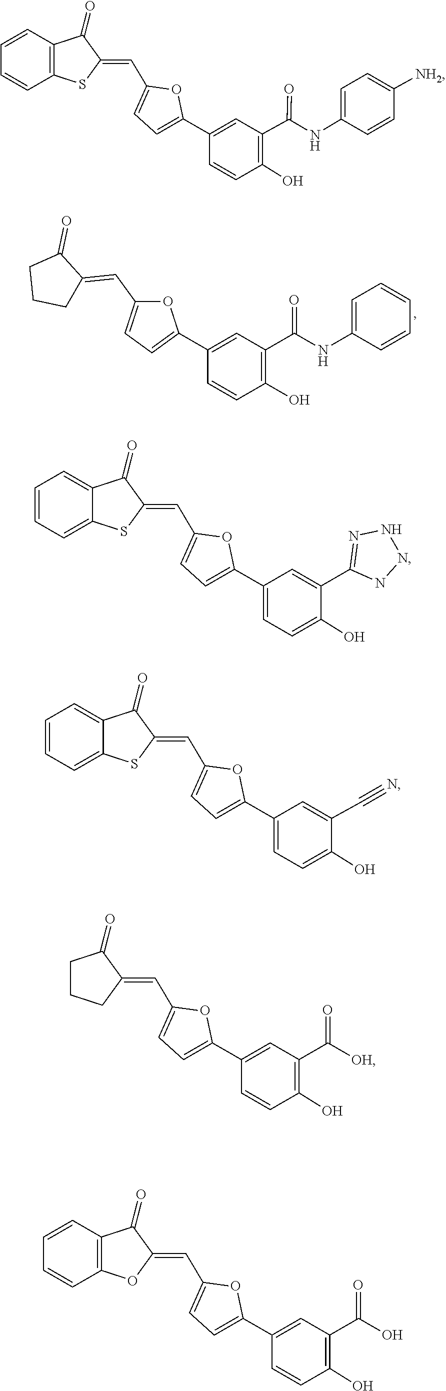

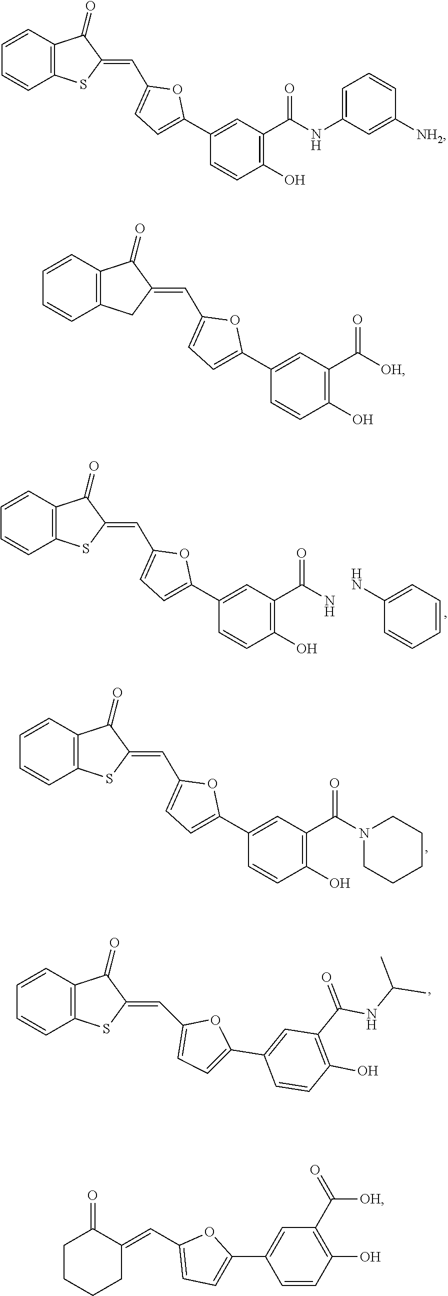

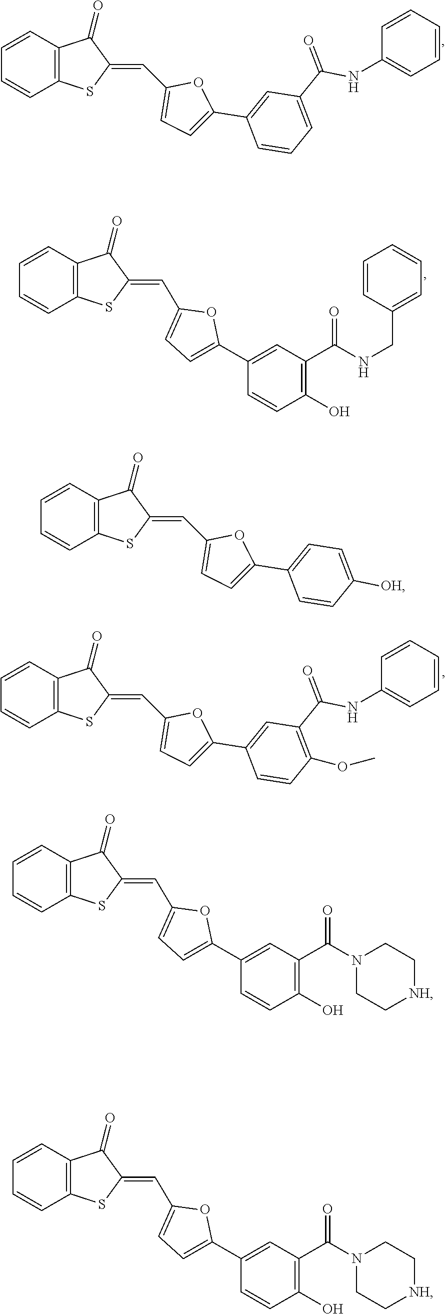 [SMILES: O=C(Nc1ccccc1)c1cccc(-c2ccc(/C=C3\Sc4ccccc4C3=O)o2)c1 ; O=C(NCc1ccccc1)c1cc(-c2ccc(/C=C3\Sc4ccccc4C3=O)o2)ccc1O ; O=C1/C(=C/c2ccc(-c3ccc(O)cc3)o2)Sc2ccccc21 ; COc1ccc(-c2ccc(/C=C3\Sc4ccccc4C3=O)o2)cc1C(=O)Nc1ccccc1 ; O=C1/C(=C/c2ccc(-c3ccc(O)c(C(=O)N4CCNCC4)c3)o2)Sc2ccccc21 ; O=C1/C(=C/c2ccc(-c3ccc(O)c(C(=O)N4CCNCC4)c3)o2)Sc2ccccc21]

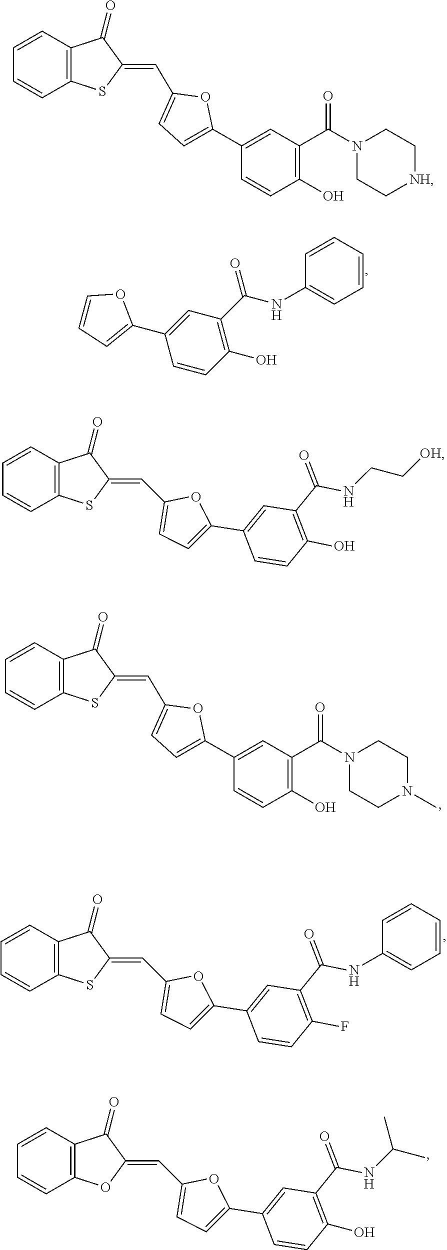

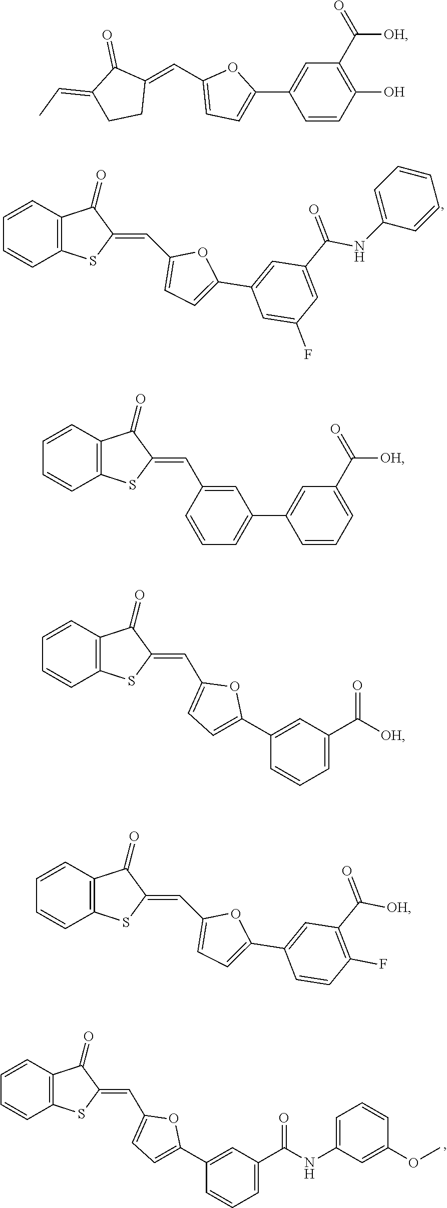 [SMILES: C/C=C1\CC/C(=C\c2ccc(-c3ccc(O)c(C(=O)O)c3)o2)C1=O ; O=C(Nc1ccccc1)c1cc(F)cc(-c2ccc(/C=C3\Sc4ccccc4C3=O)o2)c1 ; O=C(O)c1cccc(-c2cccc(/C=C3\Sc4ccccc4C3=O)c2)c1 ; O=C(O)c1cccc(-c2ccc(/C=C3\Sc4ccccc4C3=O)o2)c1 ; O=C(O)c1cc(-c2ccc(/C=C3\Sc4ccccc4C3=O)o2)ccc1F ; COc1cccc(NC(=O)c2cccc(-c3ccc(/C=C4\Sc5ccccc5C4=O)o3)c2)c1]

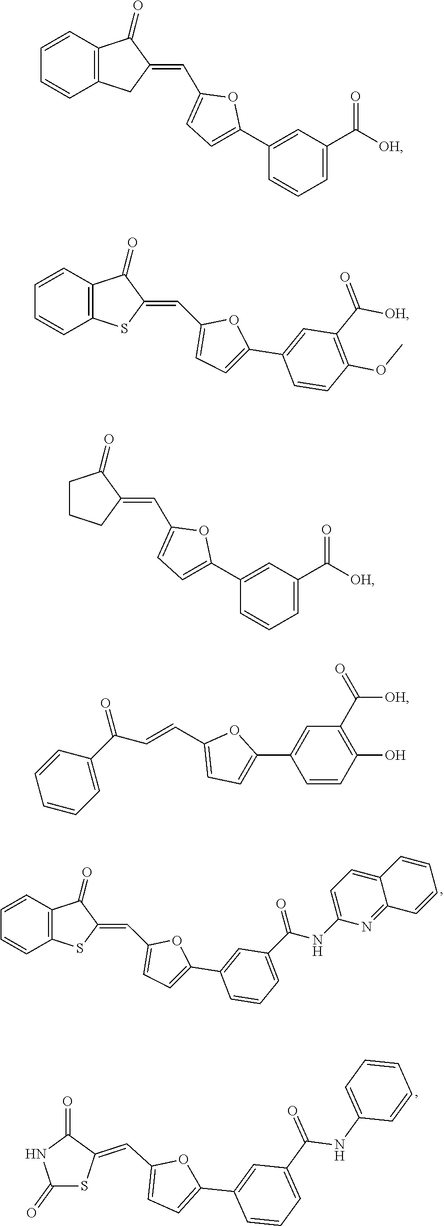 [SMILES: O=C(O)c1cccc(-c2ccc(/C=C3\Cc4ccccc4C3=O)o2)c1 ; COc1ccc(-c2ccc(/C=C3\Sc4ccccc4C3=O)o2)cc1C(=O)O ; O=C1CCC/C1=C\c1ccc(-c2cccc(C(=O)O)c2)o1 ; O=C(/C=C/c1ccc(-c2ccc(O)c(C(=O)O)c2)o1)c1ccccc1 ; O=C(Nc1ccc2ccccc2n1)c1cccc(-c2ccc(/C=C3\Sc4ccccc4C3=O)o2)c1 ; O=C1NC(=O)/C(=C/c2ccc(-c3cccc(C(=O)Nc4ccccc4)c3)o2)S1]

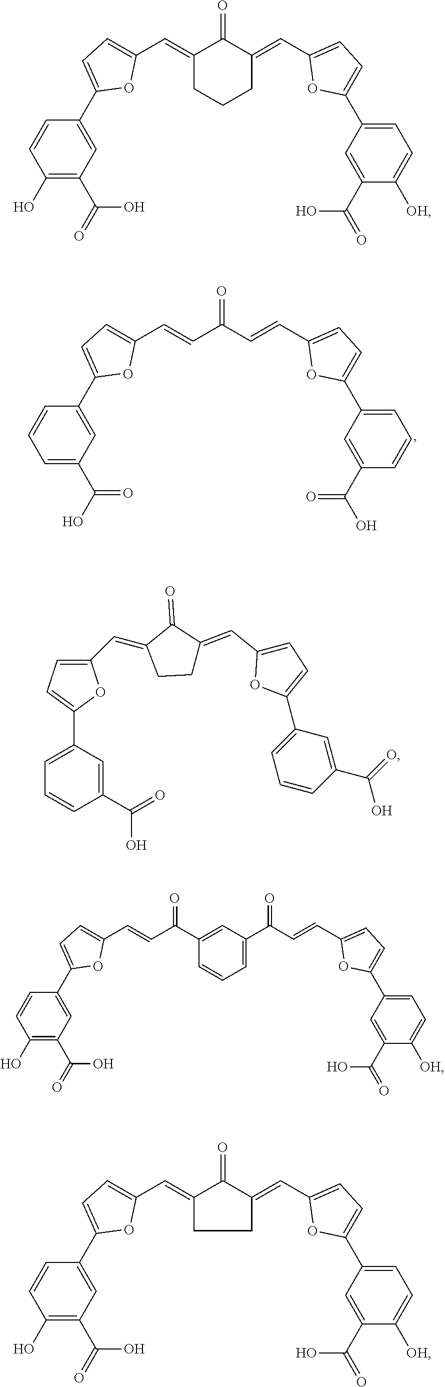 [SMILES: O=C1/C(=C/c2ccc(-c3ccc(O)c(C(=O)O)c3)o2)CCC/C1=C\c1ccc(-c2ccc(O)c(C(=O)O)c2)o1 ; O=C(/C=C/c1ccc(-c2cccc(C(=O)O)c2)o1)/C=C/c1ccc(-c2cccc(C(=O)O)c2)o1 ; O=C1/C(=C/c2ccc(-c3cccc(C(=O)O)c3)o2)CC/C1=C\c1ccc(-c2cccc(C(=O)O)c2)o1 ; O=C(/C=C/c1ccc(-c2ccc(O)c(C(=O)O)c2)o1)c1cccc(C(=O)/C=C/c2ccc(-c3ccc(O)c(C(=O)O)c3)o2)c1 ; O=C1/C(=C/c2ccc(-c3ccc(O)c(C(=O)O)c3)o2)CC/C1=C\c1ccc(-c2ccc(O)c(C(=O)O)c2)o1]

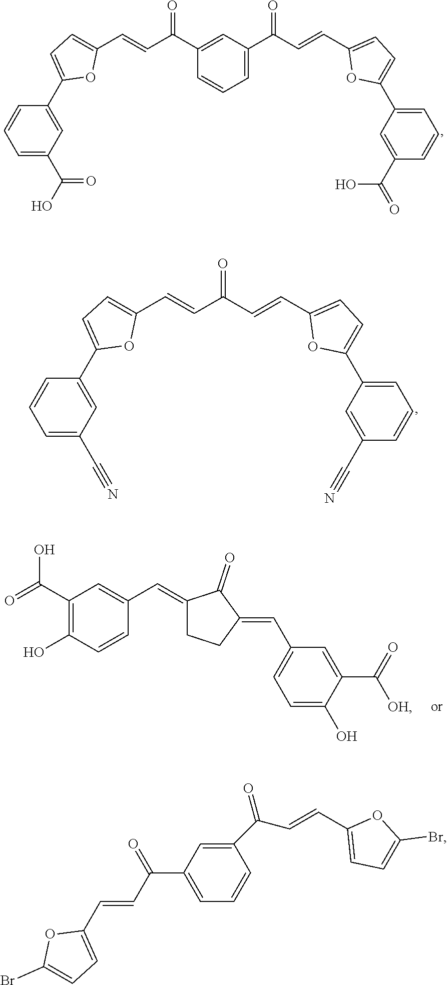 [SMILES: O=C(O)c1cccc(-c2ccc(/C=C/C(=O)c3cccc(C(=O)/C=C/c4ccc(-c5cccc(C(=O)O)c5)o4)c3)o2)c1 ; N#Cc1cccc(-c2ccc(/C=C/C(=O)/C=C/c3ccc(-c4cccc(C#N)c4)o3)o2)c1 ; O=C1/C(=C/c2ccc(O)c(C(=O)O)c2)CC/C1=C\c1ccc(O)c(C(=O)O)c1 ; O=C(/C=C/c1ccc(Br)o1)c1cccc(C(=O)/C=C/c2ccc(Br)o2)c1]

or a pharmaceutically acceptable salt thereof.

In some embodiments, R¹ is CH═Z; Z is

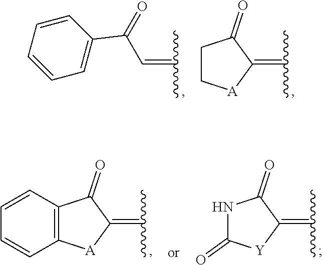

A is —CH₂—, —S—, —O—, or —CH₂—CH₂—;
Y is —O— or —S—,
R² is —CN, —C(O)N(R¹²)₂, —C(O)N(R¹²)N(R¹²)₂, —C(O)R¹² or

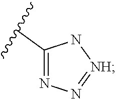

R⁴, R⁵, and R⁶ are each independently selected from hydrogen, halogen, and C₁₋₆ alkyl;
R⁷ and R⁸ are each independently selected from hydrogen, halogen, C₁₋₆ alkyl, —OR¹³, and —SR¹³;
R¹² are each independently selected from hydrogen, C₁₋₆ alkyl, C₆-C₁₀ aryl, C₃₋₆ cycloalkyl, 3-10 membered heterocycloalkyl, wherein each alkyl, aryl, cycloalkyl, or heterocycloalkyl is optionally substituted with 1-5 substituents independently selected from halogen, hydroxyl, amino, C₁₋₆ alkyl, C₁₋₆ alkoxyl, and phenyl; and
R¹³ are each independently C₁-C₆ alkyl.

In another aspect, described herein is a method of treating cancer comprising administering a therapeutically effective dose of an SHP2 inhibiting compound of Formula Ia:

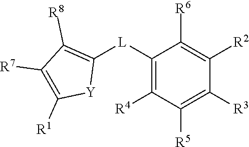

or a pharmaceutically acceptable salt thereof, wherein,
R¹ is:

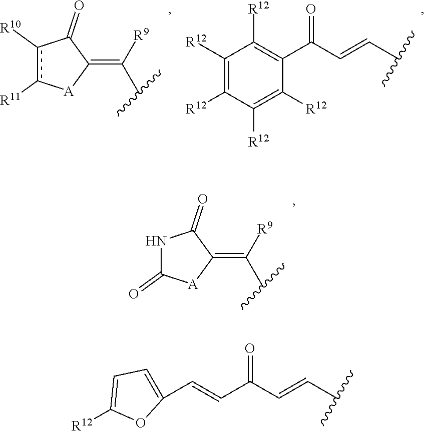

or hydrogen;
R² is selected from the group of: —H, halogens, —OH, —C═O—N(R¹³)₂, —CN, —C═O—OH, —C═O—NR¹³—N(R¹³)₂, —C═O—R¹², —C(═O)O—R¹³, —R¹², —O—R¹³, C₁-C₁₆ substituted or unsubstituted alkyl, C₁-C₁₆ substituted or unsubstituted alkenyl, substituted or unsubstituted C₆ aryl, substituted or unsubstituted C₃-C₈ cycloalkyl, substituted or unsubstituted C₃-C₈ cycloalkenyl, substituted or unsubstituted C₃-C₈ cycloalkynyl, substituted or unsubstituted heterocycloalkyl, substituted or unsubstituted heterocycloalkenyl, substituted or unsubstituted heterocycloalkynyl, C₁-C₁₆ substituted or unsubstituted alkynyl,

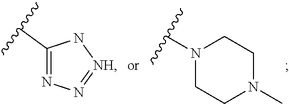

A is —CH₂—, —S—, —O—, —CH₂—CH₂—, —C(R¹²)₂—, or —C(R¹²)₂—C(R¹²)₂—;
Y is —O—, —S—, —NH— or —CH═CH—;
L is a bond, —CH═, or —C(═O)CH═CH—.

R³ is selected from the group of hydrogen, halogens, —C═O—N(R¹³)₂, —CN, —C═O—OH, —C═O—NR¹³—N(R¹³)₂, —C═O—R¹², —C(═O)O—R¹³, —R¹², —O—R¹³, —OH, —F, —Cl, —CF₃, —OCF₃, C₁-C₁₆ substituted or unsubstituted alkyl, C₁-C₁₆ substituted or unsubstituted alkenyl, substituted or unsubstituted C₆ aryl, substituted or unsubstituted C₃-C₈ cycloalkyl, substituted or unsubstituted C₃-C₈ cycloalkenyl, substituted or unsubstituted C₃-C₈ cycloalkynyl, substituted or unsubstituted heterocycloalkyl, substituted or unsubstituted heterocycloalkenyl, substituted or unsubstituted heterocycloalkynyl, or C₁-C₁₆ substituted or unsubstituted alkynyl;
R⁴, R⁵, R⁶, R⁷, R⁸, and R⁹ are each independently hydrogen, halogen, —OR¹³, —SR¹³, —N(R¹³)₂, —NO₂, —C(O)OR¹³, —C(O)N(R¹³)₂, —C(S)OR¹³, —C(S)SR¹³, —C(O)SR¹³, C₁-C₁₆ substituted or unsubstituted alkyl, C₁-C₁₆ substituted or unsubstituted alkenyl, substituted or unsubstituted C₆ aryl, substituted or unsubstituted C₃-C₈ cycloalkyl, substituted or unsubstituted C₃-C₈ cycloalkenyl, substituted or unsubstituted C₃-C₈ cycloalkynyl, substituted or unsubstituted heterocycloalkyl, substituted or unsubstituted heterocycloalkenyl, substituted or unsubstituted heterocycloalkynyl, or C₁-C₁₆ substituted or unsubstituted alkynyl;
R¹⁰ and R¹¹ either together form a substituted or unsubstituted 6 membered aryl ring, or are each independently hydrogen, —OR¹³, —SR¹³, —N(R¹³)₂, —NO₂, —CN, —C(O)OR¹³, —C(O)N(R¹³)₂, —C(S)OR¹³, —C(S)SR¹³, —C(O)SR¹³, C₁-C₁₆ substituted or unsubstituted alkyl, C₁-C₁₆ substituted or unsubstituted alkenyl, substituted or unsubstituted C₆ aryl, substituted or unsubstituted C₃-C₈ cycloalkyl, substituted or unsubstituted C₃-C₈ cycloalkenyl, substituted or unsubstituted C₃-C₈ cycloalkynyl, substituted or unsubstituted heterocycloalkyl, substituted or unsubstituted heterocycloalkenyl, substituted or unsubstituted heterocycloalkynyl, or C₁-C₁₆ substituted or unsubstituted alkynyl;
R¹² are each independently hydrogen, halogens, —OH, —CN, —SH, —NO₂,

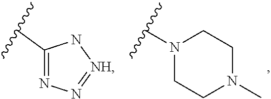

C₁-C₁₆ substituted or unsubstituted alkyl, C₁-C₁₆ substituted or unsubstituted alkyl, C₁-C₁₆ substituted or unsubstituted alkenyl, substituted or unsubstituted C aryl, substituted or unsubstituted C₃-C₈ cycloalkyl, substituted or unsubstituted C₃-C₈ cycloalkenyl, substituted or unsubstituted C₃-C₈ cycloalkynyl, substituted or unsubstituted heterocycloalkyl, substituted or unsubstituted heterocycloalkenyl, substituted or unsubstituted heterocycloalkynyl, or C₁-C₁₆ substituted or unsubstituted alkynyl;
R¹³ are each independently C₁-C₁₆ substituted or unsubstituted alkyl, C₁-C₁₆ substituted or unsubstituted alkyl, C₁-C₁₆ substituted or unsubstituted alkenyl, substituted or unsubstituted C₆ aryl, substituted or unsubstituted C₃-C₈ cycloalkyl, substituted or unsubstituted C₃-C₈ cycloalkenyl, substituted or unsubstituted C₃-C₈ cycloalkynyl, substituted or unsubstituted heterocycloalkyl, substituted or unsubstituted heterocycloalkenyl, substituted or unsubstituted heterocycloalkynyl, or C₁-C₁₆ substituted or unsubstituted alkynyl; with the proviso that the compound is not:

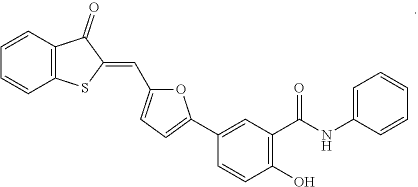 [SMILES: O=C(Nc1ccccc1)c1cc(-c2ccc(/C=C3\Sc4ccccc4C3=O)o2)ccc1O]

Some embodiments provide a method of treating cancer comprising administering a therapeutically effective dose of an SHP2 inhibiting compound to a patient in need thereof, wherein the SHP2 inhibiting compound is a compound of Formula (I) or a compound as disclosed and described herein, or a pharmaceutically acceptable salt thereof. In some embodiments, the cancer comprises cells expressing SHP2 that comprises a E76K mutation. In some embodiments, the SHP2 inhibiting compound inhibits wild type SHP2. In some embodiments, the SHP2 inhibiting compound inhibits SHP2 oncogenic variant E76K. Some embodiments provide use of a compound of Formula (I) or a compound as disclosed and described herein, or a pharmaceutically acceptable salt thereof, as a treatment for cancer or in the manufacture of a medicament for the treatment for cancer. In some embodiments, the cancer comprises cells expressing SHP2 oncogenic variant E76K.

In some embodiments of the compound of Formula Ia, R¹ is

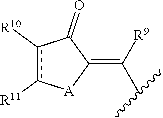

In some embodiments of the compound of Formula Ia, R⁹ is hydrogen. In some embodiments of a compound of Formula Ia, A is —O—, —S—, —CH₂—, or —CH₂CH₂—. In some embodiments of the compound of Formula Ia, R¹ is

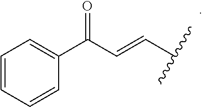 [SMILES: */C=C/C(=O)c1ccccc1]

In some embodiments of the compound of Formula Ia, R³ is —OH or a halogen. In some embodiments of the compound of Formula Ia, R² is —C(═O)—N(R¹²)₂, or —C(═O)—OR¹². In some embodiments of the compound of Formula Ia, R¹ is H. In some embodiments of the compound of Formula Ia, R³ is —OH or a halogen. In some embodiments of the compound of Formula Ia, R² is —C(═O)N(R¹²)₂. In some embodiments of the compound of Formula Ia, R³ is —OH. In some embodiments of the compound of Formula Ia, R² is —C═O—NHR¹². In some embodiments of the compound of Formula Ia, R² is

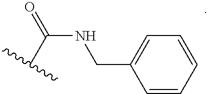 [SMILES: *C(=O)NCc1ccccc1]

In some embodiments of the compound of Formula Ia, R¹⁰ and R¹¹ together form a substituted or unsubstituted 6 membered aryl ring. In some embodiments of the compound of Formula Ia, R¹⁰ and R¹¹ are each independently selected from the group of hydrogen, —OR¹², —SR¹², —N(R¹²)₂, —NO₂, —CN, —C(═O)OR¹², —C(═O)N(R¹²)₂, —C(═S)OR¹², —C(═S)SR¹², —C(═O)SR¹², C₁-C₁₆ substituted or unsubstituted alkyl, C₁-C₁₆ substituted or unsubstituted alkenyl, substituted or unsubstituted C₆ aryl, substituted or unsubstituted C₃-C₈ cycloalkyl, substituted or unsubstituted C₃-C₈ cycloalkenyl, substituted or unsubstituted C₃-C₈ cycloalkynyl, substituted or unsubstituted heterocycloalkyl, substituted or unsubstituted heterocycloalkenyl, substituted or unsubstituted heterocycloalkynyl, or C₁-C₁₆ substituted or unsubstituted alkynyl;

In some embodiments of the compound of Formula Ia, R³ is —OH or a halogen. In some embodiments of the compound of Formula Ia, R⁹ is hydrogen. In some embodiments of the compound of Formula Ia, R⁹ is hydrogen and R³ is —OH or a halogen. In some embodiments of the compound of Formula Ia, R² is —C(═O)—N(R¹²)₂, or —C(═O)—OR¹². In some embodiments of the compound of Formula Ia, R⁹ is hydrogen and R³ is —OH or a halogen. In some embodiments of the compound of Formula Ia, R² is —C(═O)—N(R¹²)₂, or —C(═O)—OR¹².

In some embodiments of the compound of Formula Ia, the compound is selected from

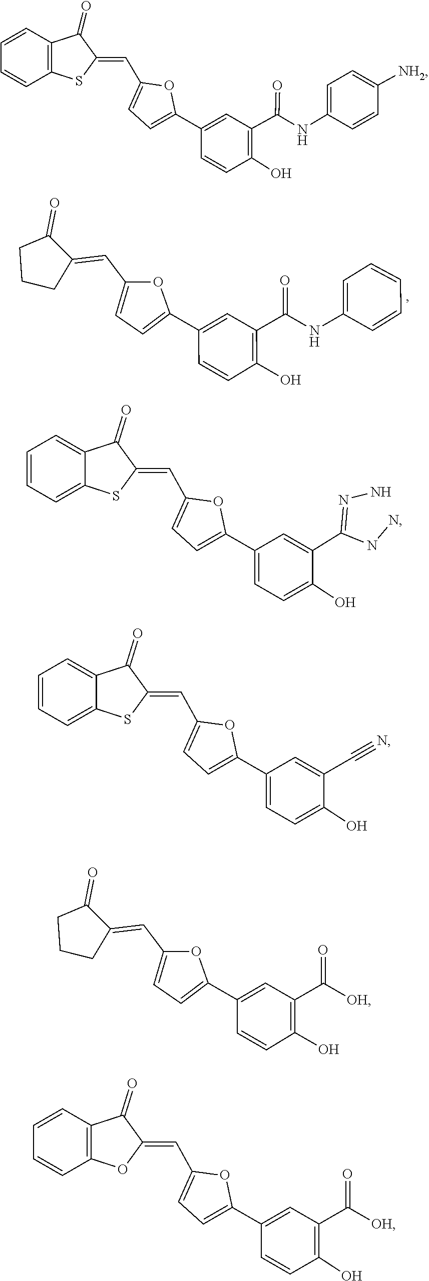

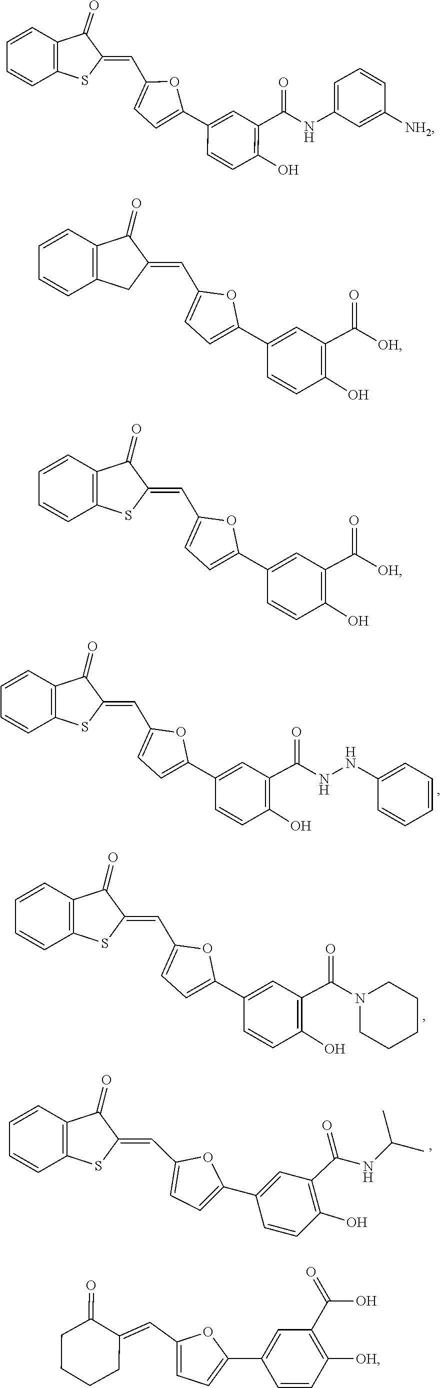

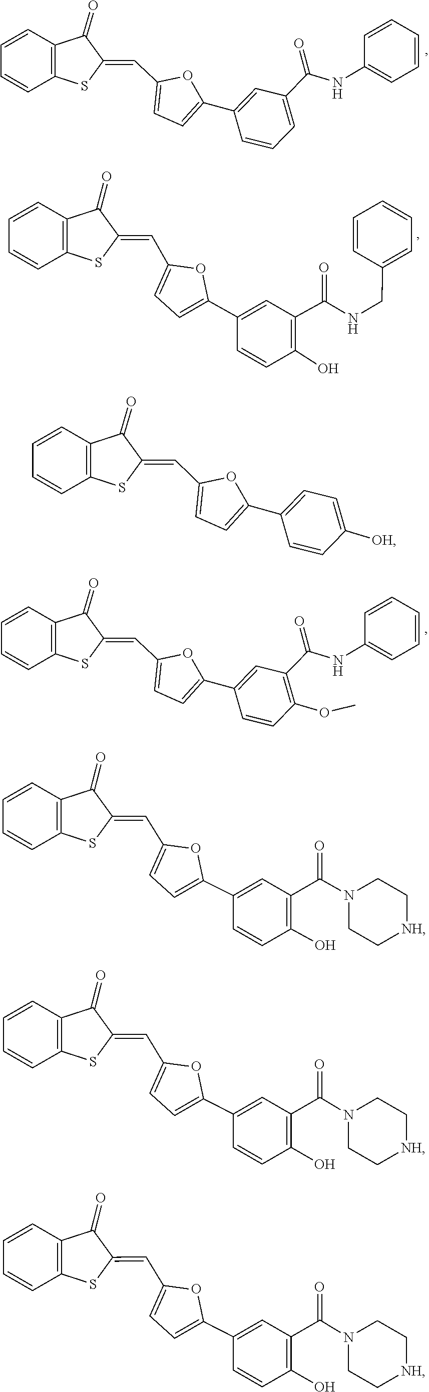

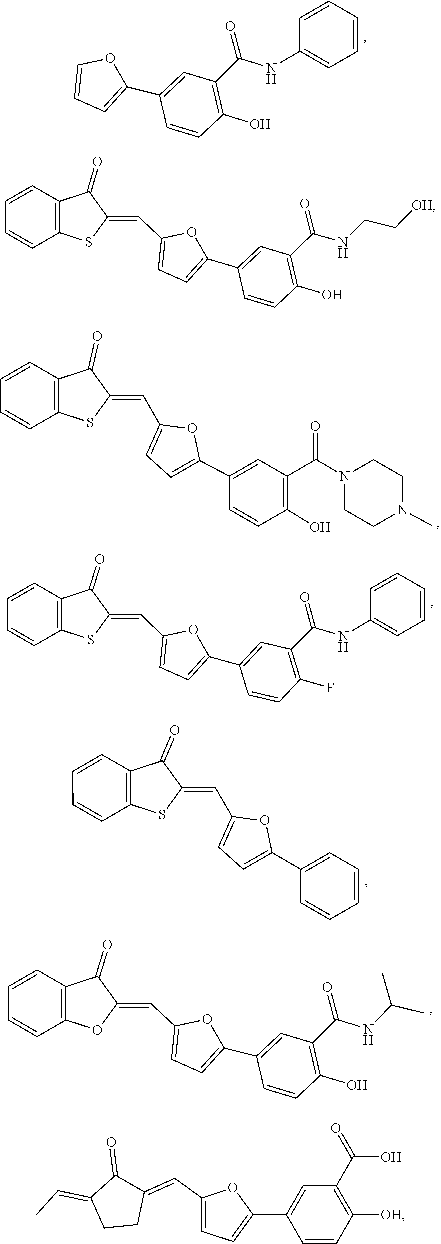

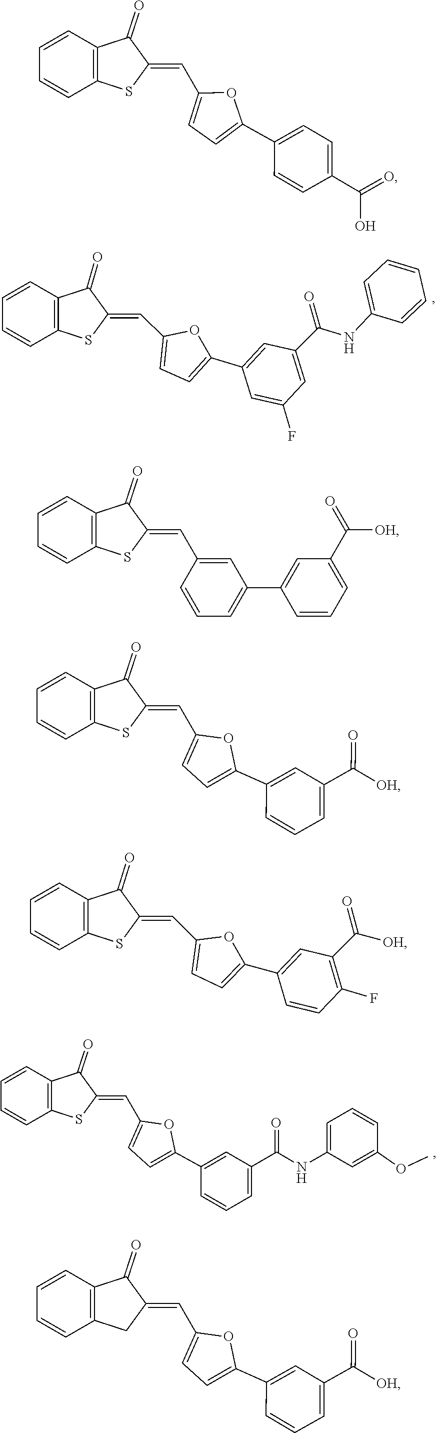

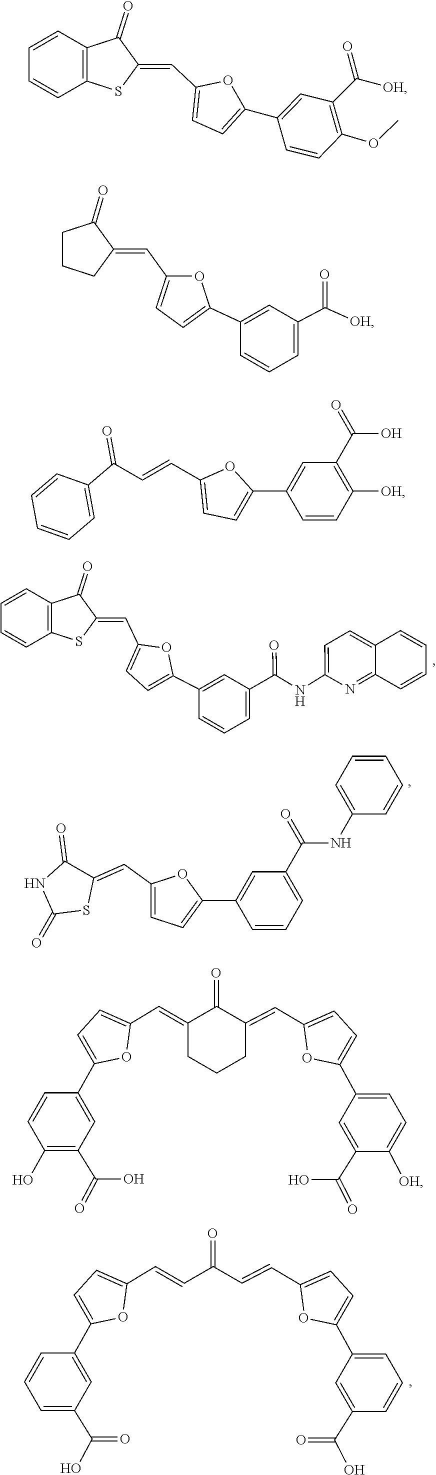

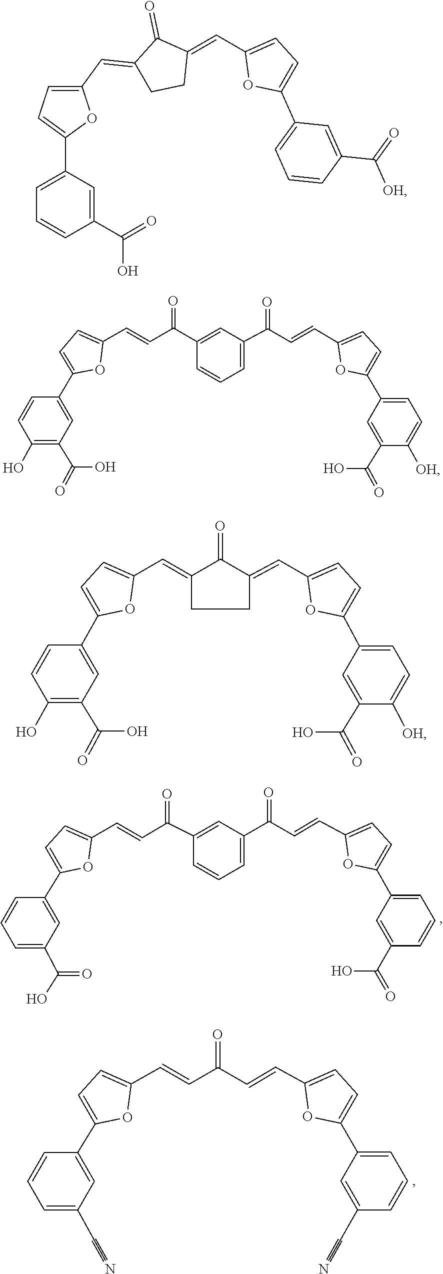 [SMILES: O=C1/C(=C/c2ccc(-c3cccc(C(=O)O)c3)o2)CC/C1=C\c1ccc(-c2cccc(C(=O)O)c2)o1 ; O=C(/C=C/c1ccc(-c2ccc(O)c(C(=O)O)c2)o1)c1cccc(C(=O)/C=C/c2ccc(-c3ccc(O)c(C(=O)O)c3)o2)c1 ; O=C1/C(=C/c2ccc(-c3ccc(O)c(C(=O)O)c3)o2)CC/C1=C\c1ccc(-c2ccc(O)c(C(=O)O)c2)o1 ; O=C(O)c1cccc(-c2ccc(/C=C/C(=O)c3cccc(C(=O)/C=C/c4ccc(-c5cccc(C(=O)O)c5)o4)c3)o2)c1 ; C=Nc1cccc(-c2ccc(/C=C/C(=O)/C=C/c3ccc(-c4cccc(N=C)c4)o3)o2)c1]

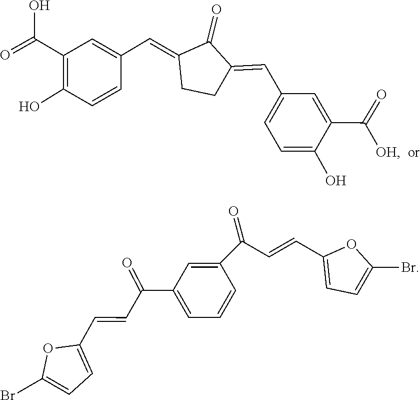

Some embodiments provide a compound selected from OH

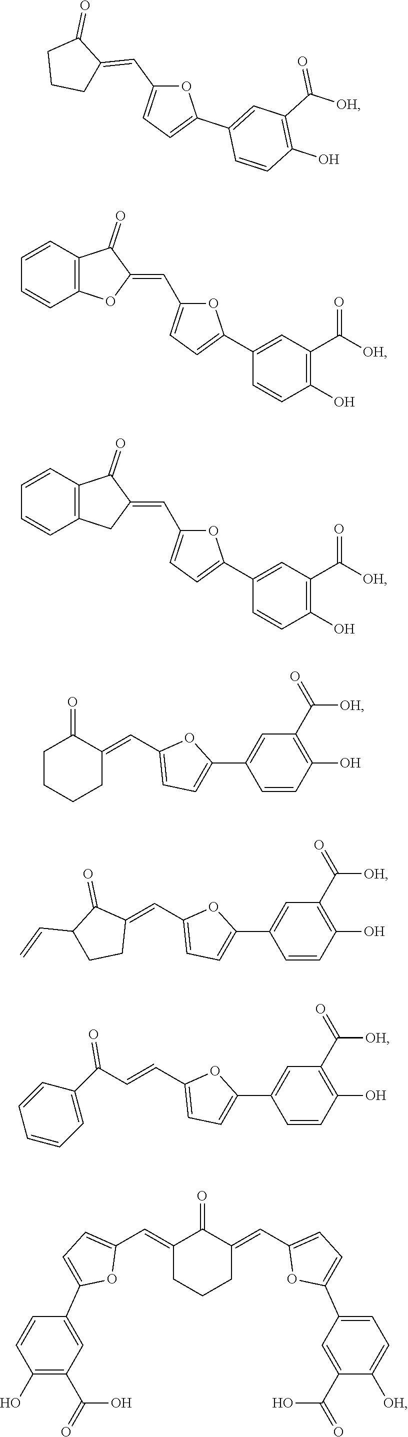

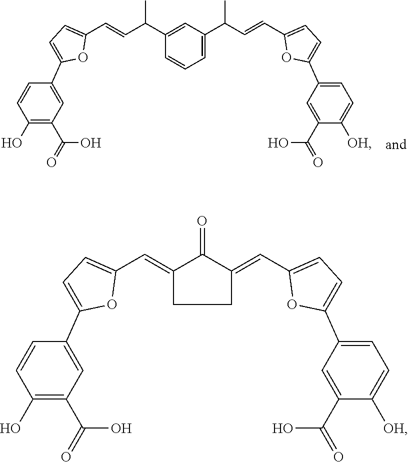 [SMILES: CC(/C=C/c1ccc(-c2ccc(O)c(C(=O)O)c2)o1)c1cccc(C(C)/C=C/c2ccc(-c3ccc(O)c(C(=O)O)c3)o2)c1 ; O=C1/C(=C/c2ccc(-c3ccc(O)c(C(=O)O)c3)o2)CC/C1=C\c1ccc(-c2ccc(O)c(C(=O)O)c2)o1]

or a pharmaceutically acceptable salt thereof.

Some embodiments provide a compound selected from

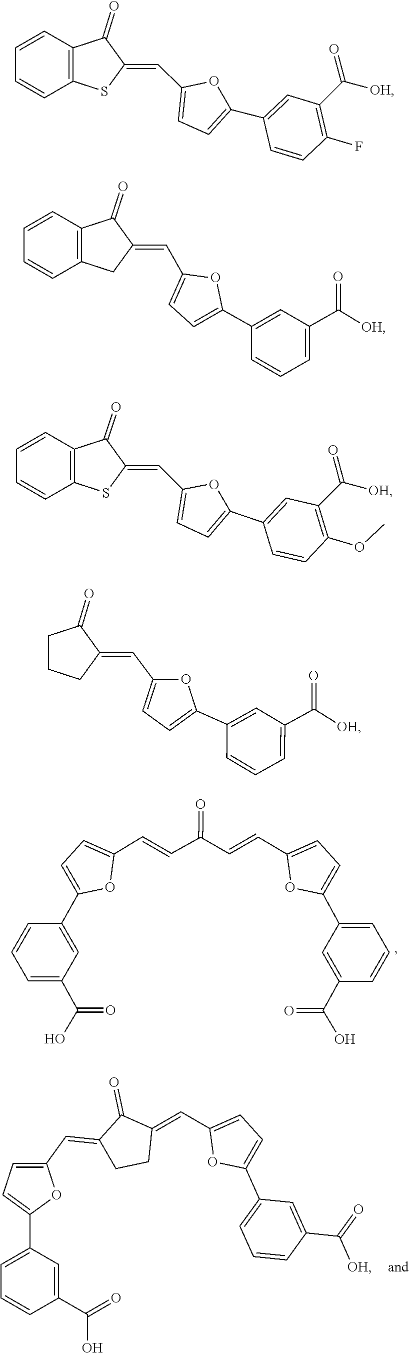 [SMILES: O=C(O)c1cc(-c2ccc(/C=C3\Sc4ccccc4C3=O)o2)ccc1F ; O=C(O)c1cccc(-c2ccc(/C=C3\Cc4ccccc4C3=O)o2)c1 ; COc1ccc(-c2ccc(/C=C3\Sc4ccccc4C3=O)o2)cc1C(=O)O ; O=C1CCC/C1=C\c1ccc(-c2cccc(C(=O)O)c2)o1 ; O=C(/C=C/c1ccc(-c2cccc(C(=O)O)c2)o1)/C=C/c1ccc(-c2cccc(C(=O)O)c2)o1 ; O=C1/C(=C/c2ccc(-c3cccc(C(=O)O)c3)o2)CC/C1=C\c1ccc(-c2cccc(C(=O)O)c2)o1]

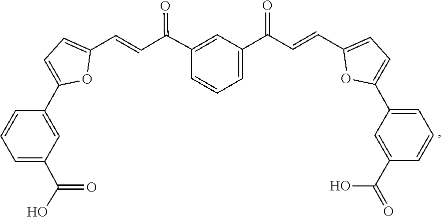 [SMILES: O=C(O)c1cccc(-c2ccc(/C=C/C(=O)c3cccc(C(=O)/C=C/c4ccc(-c5cccc(C(=O)O)c5)o4)c3)o2)c1]

a pharmaceutically acceptable salt thereof.

Some embodiments provide a compound selected from

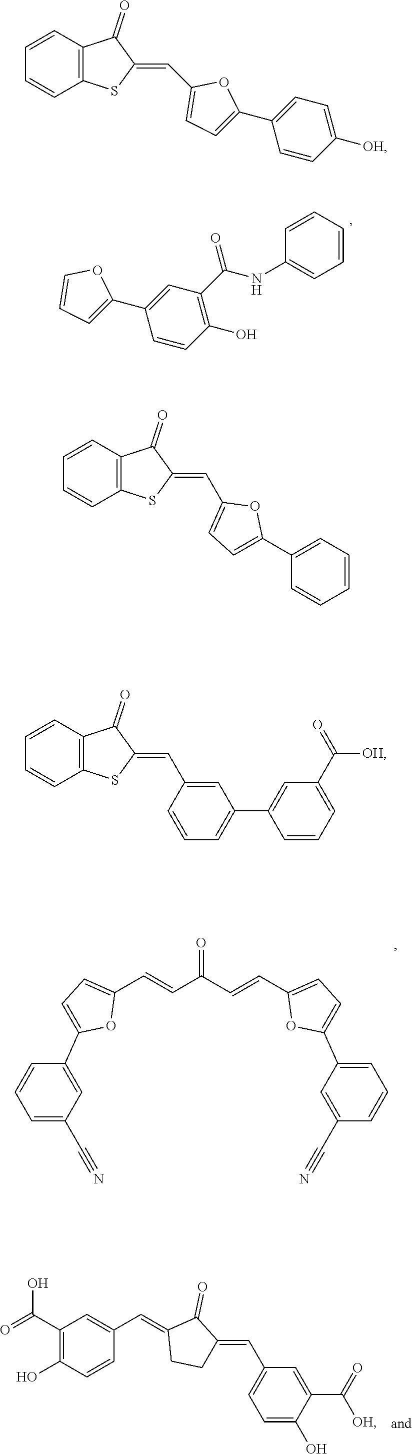 [SMILES: O=C1/C(=C/c2ccc(-c3ccc(O)cc3)o2)Sc2ccccc21 ; O=C(Nc1ccccc1)c1cc(-c2ccco2)ccc1O ; O=C1/C(=C/c2ccc(-c3ccccc3)o2)Sc2ccccc21 ; O=C(O)c1cccc(-c2cccc(/C=C3\Sc4ccccc4C3=O)c2)c1 ; N#Cc1cccc(-c2ccc(/C=C/C(=O)/C=C/c3ccc(-c4cccc(C#N)c4)o3)o2)c1 ; O=C1/C(=C/c2ccc(O)c(C(=O)O)c2)CC/C1=C\c1ccc(O)c(C(=O)O)c1]

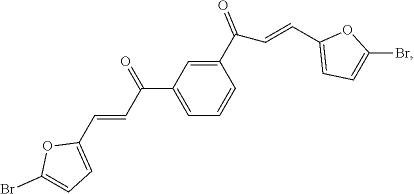 [SMILES: O=C(/C=C/c1ccc(Br)o1)c1cccc(C(=O)/C=C/c2ccc(Br)o2)c1]

or a pharmaceutically acceptable salt thereof.

Some embodiments provide a pharmaceutical composition comprising a compound or Formula (I) or a compound as disclosed and described herein, or a pharmaceutically acceptable salt thereof, and a pharmaceutically acceptable excipient. Some embodiments provide a solid form of a compound of Formula (I) or compound as disclosed and described herein, or a pharmaceutically acceptable salt thereof, or a solvate or hydrate thereof. Some embodiments provide a crystalline form a compound of Formula (I) or compound as disclosed and described herein, or a pharmaceutically acceptable salt thereof, or a solvate or hydrate thereof. Some embodiments provide an amorphous form a compound of Formula (I) or compound as disclosed and described herein, or a pharmaceutically acceptable salt thereof, or a solvate or hydrate thereof.

Any combination of the groups or variables described above or below is contemplated herein.

In some embodiments, the cancer is a blood cancer. In some embodiments, the cancer is breast cancer. In some embodiments, the cancer is leukemia. In some embodiments, the leukemia is acute lymphoblastic leukemia (ALL), acute myeloid leukemia (AML), chronic lymphocytic leukemia (CLL), chronic myeloid leukemia (CMIL), hairy cell leukemia (HCL), chronic myelomonocytic leukemia (CMML), large granular lymphocytic (LGL), blastic plasmacytoid dendritic cell neoplasm (BPDCN), B-cell prolymphocytic leukemia (B-PLL), or T-cell prolymphocytic leukemia (T-PLL). In some embodiments, the myeloma is multiple myeloma, or plasmacytoma. In some embodiments, the myeloproliferative neoplasms is myelofibrosis, polycythemia vera or essential thrombocythemia.

### BRIEF DESCRIPTION OF THE DRAWINGS

FIG. 1A, FIG. 1B, FIG. 1C, FIG. 1D, and FIG. 1E, show SHP2 regulation, inhibition, and oncogenic mutations.

FIG. 2A, FIG. 2B, FIG. 2C, FIG. 2D, and FIG. 2E show mechanism of action, inhibition, and binding studies of SHP2 inhibitors.

FIG. 3A, FIG. 3B, FIG. 3C, FIG. 3D, FIG. 3E, FIG. 3F, and FIG. 3G display evaluation of SHP2 inhibitors in wild-type SHP2 cellular cancer models and in two AML patient samples.

FIG. 4A, FIG. 4B, and FIG. 4C show evaluation of #02 (CMPD 2) in U-937 AML cells expressing the SHP2 oncogenic variant G60R.

FIG. 5A, FIG. 5B, and FIG. 5C show evaluation of SHP099, #01 (CMPD 1), and #02 (CMPD 2) in cell viability assays against mouse MLL-AF10 cells featuring wild-type SHP2 and mouse MLL-AF10 cells expressing SHP2 oncogenic variant E76K.

### DETAILED DESCRIPTION

### Definitions

The terminology used herein is for the purpose of describing particular cases only and is not intended to be limiting. In this application, the use of the singular includes the plural unless specifically stated otherwise.

As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise. Further, headings provided herein are for convenience only and do not interpret the scope or meaning of the claimed invention.

The abbreviations used herein have their conventional meaning within the chemical and biological arts, unless otherwise specified. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O—is equivalent to —OCH₂—.

The symbol “ [symbol] “denotes the point of attachment of a chemical moiety to the remainder of a molecule or chemical formula.

As used herein, the terms “about” or “approximately” can mean within an acceptable error range for the particular value as determined by one of ordinary skill in the art, which may depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, within 5-fold, or within 2-fold, of a value. Where particular values are described in the application and claims, unless otherwise stated the term “about” meaning within an acceptable error range for the particular value should be assumed.

As used herein, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the present disclosure, and vice versa. Furthermore, compositions of the present disclosure can be used to achieve methods of the present disclosure.

As used herein, the term “derivative” indicates a chemical or biological substance that is related structurally to a second substance and derivable from the second substance through a modification of the second substance. In particular, if a first compound is a derivative of a second compound and the second compound is associated with a chemical and/or biological activity, the first compound differs from the second compound for at least one structural feature, while retaining (at least to a certain extent) the chemical and/or biological activity of the second compound and at least one structural feature (e.g. a sequence, a fragment, a functional group and others) associated thereto. Non-limiting examples of “derivatives” can include a prodrug, a metabolite, an enantiomer, a diastereomer, esters (e.g. acyloxyalkyl esters, alkoxycarbonyloxyalkyl esters, alkyl esters, aryl esters, phosphate esters, sulfonate esters, sulfate esters and disulfide containing esters), ethers, amides, carbonates, thiocarbonates, N-acyl derivatives, N-acyloxyalkyl derivatives, quaternary derivatives of tertiary amines, N-Mannich bases, Schiff bases, amino acid conjugates, phosphate esters, metal salts, sulfonate esters, and the like. In some cases, a derivative may include trivial substitutions (i.e. additional alkyl/alkylene groups) to a parent compound that retains the chemical and/or biological activity of the parent compound.

As used herein, the term “pharmaceutically acceptable salt” generally refers to an acid or base salt that is generally considered in the art to be suitable for use in contact with the tissues of human beings or animals without excessive toxicity, irritation, allergic response, or other problem or complication. Such salts include mineral and organic acid salts of basic residues such as amines, as well as alkali or organic salts of acidic residues such as carboxylic acids. Specific pharmaceutical salts include, but are not limited to, salts of acids such as hydrochloric, phosphoric, hydrobromic, malic, glycolic, fumaric, sulfuric, sulfamic, sulfanilic, formic, toluenesulfonic, methanesulfonic, benzene sulfonic, ethane disulfonic, 2-hydroxyethyl sulfonic, nitric, benzoic, 2-acetoxybenzoic, citric, tartaric, lactic, stearic, salicylic, glutamic, ascorbic, pamoic, succinic, fumaric, maleic, propionic, hydroxymaleic, hydroiodic, phenylacetic, alkanoic such as acetic, HOOC—(CH₂)n-COOH where n is 0-4, and the like. Similarly, pharmaceutically acceptable cations include, but are not limited to sodium, potassium, calcium, aluminum, lithium and ammonium. Those of ordinary skill in the art will recognize from this disclosure and the knowledge in the art that further pharmaceutically acceptable salts include those listed by Remington's Pharmaceutical Sciences, 17th ed., Mack Publishing Company, Easton, PA, p. 1418 (1985). In general, a pharmaceutically acceptable acid or base salt can be synthesized from a parent compound that contains a basic or acidic moiety by any conventional chemical method. Briefly, such salts can be prepared by reacting the free acid or base forms of these compounds with a stoichiometric amount of the appropriate base or acid in an appropriate solvent.

As used herein, the term “pharmaceutically acceptable excipient, carrier or diluent” refers to an excipient, carrier or diluent that can be administered to a subject, together with an agent, and which does not destroy the pharmacological activity thereof and is nontoxic when administered in doses sufficient to deliver a therapeutic amount of the agent.

As used herein, the term “therapeutically effective amount” means an amount of an agent to be delivered (e.g., nucleic acid, drug, payload, composition, therapeutic agent, diagnostic agent, prophylactic agent, etc.) that is sufficient, when administered to a subject suffering from or susceptible to an infection, disease, disorder, and/or condition, to treat, improve symptoms of, diagnose, prevent, and/or delay the onset of the infection, disease, disorder, and/or condition.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an example range of 1 to 50 may comprise 1 to 10, 1 to 20, 1 to 30, and 1 to 40 in one direction, or 50 to 40, 50 to 30, 50 to 20, and 50 to 10 in the other direction.

As used herein, the term “subject” refers to an animal which is the object of treatment, observation, or experiment. By way of example only, a subject includes, but is not limited to, a mammal, including, but not limited to, a human or a non-human mammal, such as a non-human primate, bovine, equine, canine, ovine, or feline.

As used herein, the term “aromatic” generally refers to a planar ring having a delocalized π-electron system containing 4n+2 π electrons, where n is an integer. Aromatics can be optionally substituted. The term “aromatic” includes both aryl groups (e.g., phenyl, naphthalenyl) and heteroaryl groups (e.g., pyridinyl, quinolinyl).

As used herein, the term “Halo” or “halogen” generally refers to bromo, chloro, fluoro or iodo.

As used herein, the term “Haloalkyl” generally refers to an alkyl radical, as defined above, that is substituted by one or more halo radicals, as defined above, e.g., trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl, and the like. Unless stated otherwise specifically in the specification, a haloalkyl group may be optionally substituted.

As used herein, the term “Haloalkoxy” generally refers to an alkoxy radical, as defined above, that is substituted by one or more halo radicals, as defined above, e.g., trifluoromethoxy, difluoromethoxy, fluoromethoxy, trichloromethoxy, 2,2,2-trifluoroethoxy, 1,2-difluoroethoxy, 3-bromo-2-fluoropropoxy, 1,2-dibromoethoxy, and the like. Unless stated otherwise specifically in the specification, a haloalkoxy group may be optionally substituted.

As used herein, the term “tautomer” generally refers to a proton shift from one atom of a molecule to another atom of the same molecule. The compounds presented herein may exist as tautomers. Tautomers are compounds that are interconvertible by migration of a hydrogen atom, accompanied by a switch of a single bond and adjacent double bond. In bonding arrangements where tautomerization is possible, a chemical equilibrium of the tautomers may exist. All tautomeric forms of the compounds disclosed herein are contemplated. The exact ratio of the tautomers depends on several factors, including temperature, solvent, and pH. Some examples of tautomeric interconversions include:

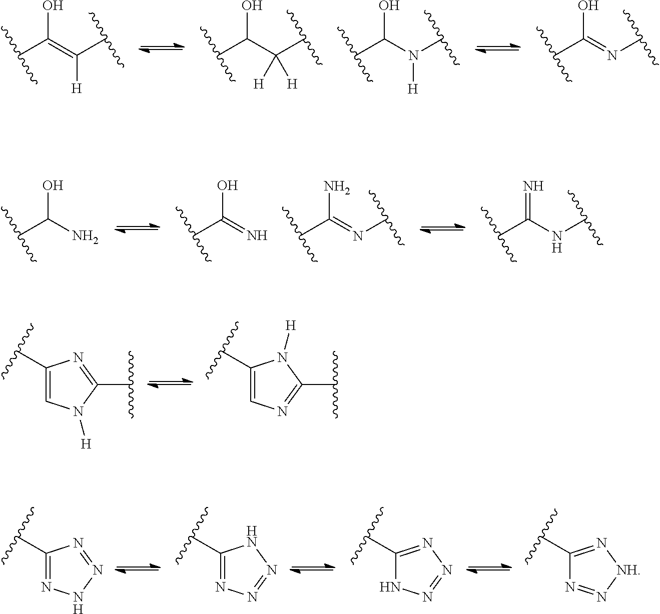

As used herein, the terms “co-administration” or the like, may encompass administration of the selected therapeutic agents to a single patient, and are intended to include treatment regimens in which the agents are administered by the same or different route of administration or at the same or different time.

As used herein, the terms “effective amount” or “therapeutically effective amount,” generally refer to a sufficient amount of an agent or a compound being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case may be determined using techniques, such as a dose escalation study. An “effective amount” is an amount sufficient for a compound to accomplish a stated purpose relative to the absence of the compound (e.g., achieve the effect for which it is administered, treat a disease, reduce enzyme activity, increase enzyme activity, reduce a signaling pathway, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist. The exact amounts may depend on the purpose of the treatment, and may be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

As used herein, the term “substituted”, unless otherwise indicated, refers to the replacement of one or more hydrogen radicals in a given structure with the radical of a specified substituent including, but not limited to: halo, alkyl, alkenyl, alkynyl, aryl, heterocyclyl, thiol, alkylthio, oxo, thioxy, arylthio, alkylthioalkyl, arylthioalkyl, alkylsulfonyl, alkylsulfonylalkyl, arylsulfonylalkyl, alkoxy, aryloxy, aralkoxy, aminocarbonyl, alkylaminocarbonyl, arylaminocarbonyl, alkoxycarbonyl, aryloxycarbonyl, haloalkyl, amino, trifluoromethyl, cyano, nitro, alkylamino, arylamino, alkylaminoalkyl, arylaminoalkyl, aminoalkylamino, hydroxy, alkoxyalkyl, carboxyalkyl, alkoxycarbonylalkyl, aminocarbonylalkyl, acyl, aralkoxycarbonyl, carboxylic acid, sulfonic acid, sulfonyl, phosphonic acid, aryl, heteroaryl, heterocyclic, and an aliphatic group. It is understood that the substituent may be further substituted. Example substituents include amino, alkylamino, and the like.

As used herein, the term “substituent” generally refers to positional variables on the atoms of a core molecule that are substituted at a designated atom position, replacing one or more hydrogens on the designated atom, provided that the designated atom's normal valency is not exceeded, and that the substitution results in a stable compound. Combinations of substituents and/or variables are permissible only if such combinations result in stable compounds. A person of ordinary skill in the art should note that any carbon as well as heteroatom with valences that appear to be unsatisfied as described or shown herein is assumed to have a sufficient number of hydrogen atom(s) to satisfy the valences described or shown. In certain instances one or more substituents having a double bond (e.g., “oxo” or “═O”) as the point of attachment may be described, shown or listed herein within a substituent group, wherein the structure may only show a single bond as the point of attachment to the core structure of Formula (I). A person of ordinary skill in the art would understand that, while only a single bond is shown, a double bond is intended for those substituents.

As used herein, the term “alkyl” generally refers to a straight or branched hydrocarbon chain radical, having from one to twenty carbon atoms, and which is attached to the rest of the molecule by a single bond. An alkyl comprising up to 10 carbon atoms is referred to as a C₁-C₁₀ alkyl, likewise, for example, an alkyl comprising up to 6 carbon atoms is a C₁-C₆ alkyl. Alkyls (and other moieties defined herein) comprising other numbers of carbon atoms are represented similarly. Alkyl groups include, but are not limited to, C₁-C₁₀ alkyl, C₁-C₉ alkyl, C₁-C₈ alkyl, C₁-C₇ alkyl, C₁-C₆ alkyl, C₁-C₅ alkyl, C₁-C₄ alkyl, C₁-C₃ alkyl, C₁-C₂ alkyl, C₂-C₈ alkyl, C₃-C₈ alkyl and C₄-C₈ alkyl. Representative alkyl groups include, but are not limited to, methyl, ethyl, n-propyl, 1-methylethyl (i-propyl), n-butyl, i-butyl, s-butyl, n-pentyl, 1,1-dimethylethyl (t-butyl), 3-methylhexyl, 2-methylhexyl, 1-ethyl-propyl, and the like. In some embodiments, the alkyl is methyl or ethyl. In some embodiments, the alkyl is —CH(CH₃)₂ or —C(CH₃)₃. Unless stated otherwise specifically in the specification, an alkyl group may be optionally substituted as described below. “Alkylene” or “alkylene chain” refers to a straight or branched divalent hydrocarbon chain linking the rest of the molecule to a radical group. In some embodiments, the alkylene is —CH₂—, —CH₂CH₂—, or —CH₂CH₂CH₂—. In some embodiments, the alkylene is —CH₂—. In some embodiments, the alkylene is —CH₂CH₂—. In some embodiments, the alkylene is —CH₂CH₂CH₂—.

As used herein, the term “aryl” refers to a radical derived from a hydrocarbon ring system comprising at least one aromatic ring. In some embodiments, an aryl comprises hydrogens and 6 to 30 carbon atoms. The aryl radical can be a monocyclic, bicyclic, tricyclic, or tetracyclic ring system, which may include fused (when fused with a cycloalkyl or heterocycloalkyl ring, the aryl is bonded through an aromatic ring atom) or bridged ring systems. In some embodiments, the aryl is a 6- to 10-membered aryl. In some embodiments, the aryl is a 6-membered aryl. Aryl radicals include, but are not limited to, aryl radicals derived from the hydrocarbon ring systems of anthrylene, naphthylene, phenanthrylene, anthracene, azulene, benzene, chrysene, fluoranthene, fluorene, indane, indene, naphthalene, phenalene, phenanthrene, pleiadene, pyrene, and triphenylene. In some embodiments, the aryl is phenyl. Unless stated otherwise specifically in the specification, an aryl can be optionally substituted, for example, with halogen, amino, alkylamino, aminoalkyl, nitrile, nitro, hydroxyl, alkyl, alkenyl, alkynyl, haloalkyl, heteroalkyl, alkoxy, aryl, cycloalkyl, heterocycloalkyl, heteroaryl, —S(O)₂NH—C₁-C₆alkyl, and the like. In some embodiments, an aryl is optionally substituted with halogen, methyl, ethyl, —CN, —CF₃, —OH, —OMe, —NH₂, —NO₂, —S(O)₂NH₂, —S(O)₂NHCH₃, —S(O)₂NHCH₂CH₃, —S(O)₂NHCH(CH₃)₂, —S(O)₂N(CH₃)₂, or —S(O)₂NHC(CH₃)₃. In some embodiments, an aryl is optionally substituted with halogen, methyl, ethyl, —CN, —CF₃, —OH, or —OMe. In some embodiments, the aryl is optionally substituted with halogen. In some embodiments, the aryl is substituted with alkyl, alkenyl, alkynyl, haloalkyl, or heteroalkyl, wherein each alkyl, alkenyl, alkynyl, haloalkyl, heteroalkyl is independently unsubstituted, or substituted with halogen, methyl, ethyl, —CN, —CF₃, —OH, —OMe, —NH₂, or —NO₂.

As used herein, the term “alkenyl” generally refers to a type of alkyl group in which at least one carbon-carbon double bond is present. In one embodiment, an alkenyl group has the formula —C(R^{a})═CR^{a}₂, wherein R^{a} refers to the remaining portions of the alkenyl group, which may be the same or different. In some embodiments, R^{a} is H or an alkyl. In some embodiments, an alkenyl is selected from ethenyl (i.e., vinyl), propenyl (i.e., allyl), butenyl, pentenyl, pentadienyl, and the like. Non-limiting examples of an alkenyl group include —CH═CH₂, —C(CH₃)═CH₂, —CH═CHCH₃, —C(CH₃)═CHCH₃, and —CH₂CH═CH₂. “Alkenylene” or “alkenylene chain” refers to a alkylene group in which at least one carbon-carbon double bond is present. In some embodiments, the alkenylene is —CH═CH—, —CH₂CH₂CH═CH—, or —CH═CHCH₂CH₂—. In some embodiments, the alkenylene is —CH═CH—. In some embodiments, the alkenylene is —CH₂CH₂CH═CH—. In some embodiments, the alkenylene is —CH═CHCH₂CH₂—.

As used herein, the term “alkynyl” generally refers to a type of alkyl group in which at least one carbon-carbon triple bond is present. In one embodiment, an alkynyl group has the formula —C≡CR^{a}, wherein R^{a} refers to the remaining portions of the alkynyl group. In some embodiments, R^{a} is H or an alkyl. In some embodiments, an alkynyl is selected from ethynyl (i.e., acetylenyl), propynyl (i.e., propargyl), butynyl, pentynyl, and the like. Non-limiting examples of an alkynyl group include —C≡CH, —C≡CCH₃, and —CH₂C≡CH. “Alkynylene” or “alkynylene chain” refers to a alkylene group in which at least one carbon-carbon triple bond is present. In some embodiments, the alkynylene is —C≡C—, —CH₂CH₂C≡C—, or —C≡CCH₂CH₂—. In some embodiments, the alkynylene is —C≡C—. In some embodiments, the alkynylene is —CH₂CH₂C≡C—. In some embodiments, the alkynylene is —C≡CCH₂CH₂—.

As used herein, the term “cycloalkyl” generally refers to a monocyclic or polycyclic non-aromatic radical, wherein each of the atoms forming the ring (i.e. skeletal atoms) is a carbon atom. In some embodiments, cycloalkyls are saturated or partially unsaturated. In some embodiments, cycloalkyls are spirocyclic or bridged compounds. In some embodiments, cycloalkyls are fused with an aromatic ring (in which case the cycloalkyl is bonded through a non-aromatic ring carbon atom). Cycloalkyl groups include groups having from 3 to 10 ring atoms. Representative cycloalkyls include, but are not limited to, cycloalkyls having from three to ten carbon atoms, from three to eight carbon atoms, from three to six carbon atoms, or from three to five carbon atoms. Monocyclic cycloalkyl radicals include, for example, cyclopropyl, cyclobutyl, cyclopentyl, cyclohexyl, cycloheptyl, and cyclooctyl. In some embodiments, the monocyclic cycloalkyl is cyclopropyl, cyclobutyl, cyclopentyl or cyclohexyl. In some embodiments, the monocyclic cycloalkyl is cyclopentenyl or cyclohexenyl. In some embodiments, the monocyclic cycloalkyl is cyclopentenyl. Polycyclic radicals include, for example, adamantyl, 1,2-dihydronaphthalenyl, 1,4-dihydronaphthalenyl, tetrainyl, decalinyl, 3,4-dihydronaphthalenyl-1(2H)-one, spiro[2.2]pentyl, norbornyl and bicycle[1.1.1]pentyl. Unless otherwise stated specifically in the specification, a cycloalkyl group may be optionally substituted. Depending on the structure, a cycloalkyl group can be monovalent or divalent (i.e., a cycloalkylene group).

As used herein, the term “heterocycle” or “heterocyclic” generally refers to heteroaromatic rings (also known as heteroaryls) and heterocycloalkyl rings (also known as heteroalicyclic groups) that includes at least one heteroatom selected from nitrogen, oxygen and sulfur, wherein each heterocyclic group has from 3 to 12 atoms in its ring system, and with the proviso that any ring does not contain two adjacent O or S atoms. A “heterocyclyl” is a univalent group formed by removing a hydrogen atom from any ring atoms of a heterocyclic compound. In some embodiments, heterocycles are monocyclic, bicyclic, polycyclic, spirocyclic or bridged compounds. Non-aromatic heterocyclic groups (also known as heterocycloalkyls) include rings having 3 to 12 atoms in its ring system and aromatic heterocyclic groups include rings having 5 to 12 atoms in its ring system. The heterocyclic groups include benzo-fused ring systems. Examples of non-aromatic heterocyclic groups are pyrrolidinyl, tetrahydrofuranyl, dihydrofuranyl, tetrahydrothienyl, oxazolidinonyl, tetrahydropyranyl, dihydropyranyl, tetrahydrothiopyranyl, piperidinyl, morpholinyl, thiomorpholinyl, thioxanyl, piperazinyl, aziridinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, pyrrolin-2-yl, pyrrolin-3-yl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, ₃h-indolyl, indolin-2-onyl, isoindolin-1-onyl, isoindoline-1,3-dionyl, 3,4-dihydroisoquinolin-1(2H)-onyl, 3,4-dihydroquinolin-2(1H)-onyl, isoindoline-1,3-dithionyl,benzo[d]oxazol-2(3H)-onyl, 1H-benzo[d]imidazol-2(3H)-onyl, benzo[d]thiazol-2(3H)-onyl, and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups are either C-attached (or C-linked) or N-attached where such is possible. For instance, a group derived from pyrrole includes both pyrrol-1-yl (N-attached) or pyrrol-3-yl (C-attached). Further, a group derived from imidazole includes imidazol-1-yl or imidazol-3-yl (both N-attached) or imidazol-2-yl, imidazol-4-yl or imidazol-5-yl (all C-attached). The heterocyclic groups include benzo-fused ring systems. Non-aromatic heterocycles are optionally substituted with one or two oxo (═O) moieties, such as pyrrolidin-2-one. In some embodiments, at least one of the two rings of a bicyclic heterocycle is aromatic. In some embodiments, both rings of a bicyclic heterocycle are aromatic.

As used herein, the term “heterocycloalkyl” generally refers to a cycloalkyl group that includes at least one ring heteroatom selected from nitrogen, oxygen, and sulfur. Unless stated otherwise specifically in the specification, the heterocycloalkyl radical may be a monocyclic, or bicyclic ring system, which may include fused (when fused with an aryl or a heteroaryl ring, the heterocycloalkyl is bonded through a non-aromatic ring atom) or bridged ring systems. The nitrogen, carbon or sulfur atoms in the heterocyclyl radical may be optionally oxidized. The nitrogen atom may be optionally quaternized. The heterocycloalkyl radical is partially or fully saturated. Examples of heterocycloalkyl radicals include, but are not limited to, dioxolanyl, thienyl[1,3]dithianyl, tetrahydroquinolyl, tetrahydroisoquinolyl, decahydroquinolyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl. The term heterocycloalkyl also includes all ring forms of carbohydrates, including but not limited to monosaccharides, disaccharides and oligosaccharides. In some embodiments, heterocycloalkyls have from 2 to 10 carbons in the ring. In some embodiments, heterocycloalkyls have from 2 to 10 carbons in the ring and 1 or 2 N atoms. In some embodiments, heterocycloalkyls have from 2 to 10 carbons in the ring and 3 or 4 N atoms. In some embodiments, heterocycloalkyls have from 2 to 12 carbons, 0-2 N atoms, 0-2 O atoms, 0-2 P atoms, and 0-2 S atoms in the ring. In some embodiments, heterocycloalkyls have from 2 to 12 carbons, 1-3 N atoms, 0-2 O atoms, and 0-2 S atoms in the ring. It is understood that when referring to the number of carbon atoms in a heterocycloalkyl, the number of carbon atoms in the heterocycloalkyl is not the same as the total number of atoms (including the heteroatoms) that makeup the heterocycloalkyl (i.e. skeletal atoms of the heterocycloalkyl ring). Unless stated otherwise specifically in the specification, a heterocycloalkyl group may be optionally substituted. As used herein, the term “heterocycloalkylene” can refer to a divalent heterocycloalkyl group.

As used herein, the term “heteroaryl” generally refers to an aryl group that includes one or more ring heteroatoms selected from nitrogen, oxygen and sulfur. The heteroaryl is monocyclic or bicyclic. Illustrative examples of monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, furazanyl, indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. Illustrative examples of monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, and furazanyl. Illustrative examples of bicyclic heteroaryls include indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. In some embodiments, heteroaryl is pyridinyl, pyrazinyl, pyrimidinyl, thiazolyl, thienyl, thiadiazolyl or furyl. In some embodiments, a heteroaryl contains 0-6 N atoms in the ring. In some embodiments, a heteroaryl contains 1-4 N atoms in the ring. In some embodiments, a heteroaryl contains 4-6 N atoms in the ring. In some embodiments, a heteroaryl contains 0-4N atoms, 0-1 O atoms, 0-1 P atoms, and 0-1 S atoms in the ring. In some embodiments, a heteroaryl contains 1-4 N atoms, 0-1 O atoms, and 0-1 S atoms in the ring. In some embodiments, heteroaryl is a C₁-C₉heteroaryl. In some embodiments, monocyclic heteroaryl is a C₁-C₅ heteroaryl. In some embodiments, monocyclic heteroaryl is a 5-membered or 6-membered heteroaryl. In some embodiments, a bicyclic heteroaryl is a C₆-C₉ heteroaryl. In some embodiments, a heteroaryl group is partially reduced to form a heterocycloalkyl group defined herein. In some embodiments, a heteroaryl group is fully reduced to form a heterocycloalkyl group defined herein.

As used herein, the term “heteroalkyl” generally refers to an alkyl group in which one or more skeletal atoms of the alkyl are selected from an atom other than carbon, e.g., oxygen, nitrogen (e.g. —NH—, —N(alkyl)-, or —N(aryl)-), sulfur (e.g. —S—, —S(═O)—, or —S(═O)₂—), or combinations thereof. In some embodiments, a heteroalkyl is attached to the rest of the molecule at a carbon atom of the heteroalkyl. In some embodiments, a heteroalkyl is attached to the rest of the molecule at a heteroatom of the heteroalkyl. In some embodiments, a heteroalkyl is a C₁-C₆ heteroalkyl. Representative heteroalkyl groups include, but are not limited to —OCH₂OMe, —OCH₂CH₂OH, —OCH₂CH₂OMe, or —OCH₂CH₂OCH₂CH₂NH₂. “Heteroalkylene” or “heteroalkylene chain” refers to a straight or branched divalent heteroalkyl chain linking the rest of the molecule to a radical group. Unless stated otherwise specifically in the specification, the heteroalkyl or heteroalkylene group may be optionally substituted. Representative heteroalkylene groups include, but are not limited to —OCH₂CH₂O—, —OCH₂CH₂OCH₂CH₂O—, or —OCH₂CH₂OCH₂CH₂OCH₂CH₂O—.

As used herein, the term “heteroalkenyl” refers to an alkenyl group in which one or more skeletal atoms of the alkenyl are selected from an atom other than carbon, e.g., oxygen, nitrogen (e.g. —NH—, —N(alkyl)-, or —N(aryl)-), sulfur (e.g. —S—, —S(═O)—, or —S(═O)₂—), or combinations thereof. In some embodiments, a heteroalkenyl is attached to the rest of the molecule at a carbon atom of the heteroalkenyl. In some embodiments, a heteroalkenyl is attached to the rest of the molecule at a heteroatom of the heteroalkenyl. In some embodiments, a heteroalkyl is a C₁-C₆ heteroalkenyl.

As used herein, the term “heteroalkynyl” refers to an alkynyl group in which one or more skeletal atoms of the alkynyl are selected from an atom other than carbon, e.g., oxygen, nitrogen (e.g. —NH—, —N(alkyl)-, or —N(aryl)-), sulfur (e.g. —S—, —S(═O)—, or —S(═O)₂—), or combinations thereof. In some embodiments, a heteroalkynyl is attached to the rest of the molecule at a carbon atom of the heteroalkynyl. In some embodiments, a heteroalkynyl is attached to the rest of the molecule at a heteroatom of the heteroalkynyl. In some embodiments, a heteroalkyl is a C₁-C₆ heteroalkynyl.

As used herein, the term “heteroatom” or “ring heteroatom” generally refers to an atom including oxygen (O), nitrogen (N), sulfur (S), phosphorus (P), and silicon (Si), or any combination thereof.

As used herein, the term “substituent group,” refers to a group selected from the following moieties:
(A) oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, unsubstituted heteroaryl, and
(B) alkyl, heteroalkyl, cycloalkyl, heterocycloalkyl, aryl, heteroaryl, substituted with at least one substituent selected from:
(i) oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, unsubstituted heteroaryl, and
(ii) alkyl, heteroalkyl, cycloalkyl, heterocycloalkyl, aryl, heteroaryl, substituted with at least one substituent selected from:
(a) oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, unsubstituted heteroaryl, and
(b) alkyl, heteroalkyl, cycloalkyl, heterocycloalkyl, aryl, heteroaryl, substituted with at least one substituent selected from: oxo, halogen, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, unsubstituted heteroaryl.

In some embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

### SHP2 Inhibitors

SHP2 may be an attractive therapeutic target for cancers with upregulated RTK and Ras signaling, either as a monotherapy or in combination with other RTK/Ras pathway and immune checkpoint inhibitors. The new class of SHP2 allosteric inhibitors may ultimately prove to be transformative for the treatment of many cancers. However, because of the unique mechanism of action, they may likely be ineffective for patients with cancers that are driven by oncogenic mutant forms of SHP2, most notably pediatric and acute leukemias. Among all blood cancers, the highest rates of SHP2 gain-of-function mutations occur in juvenile myelomonocytic leukemia (JMML), with leukemogenesis driven by SHP2 variants in up to 42% of these children (source: COSMIC database, March 2021). Acute leukemias with larger patient populations such as AML and B cell acute lymphoblastic leukemia (B-ALL) have SHP2 mutation rates between 5 and 10%, and those mutations are associated with poor clinical outcomes as well as resistance to targeted therapies (46,47). Finally, cancers driven by aberrant FGF signaling, such as many breast cancers, seem to be inherently resistant to current SHP2 allosteric inhibitors (34). The reason for this resistance is a rapid feedback activation of the FGF receptor that leads to increased recruitment and activation of SHP2. Clearly, there is an urgent need for novel SHP2 inhibitory compounds that act through different mechanisms and exhibit a new inhibition paradigm.

Herein described is the synthesis and SAR of a series of furanylbenzamides that bind and inhibit both or either of oncogenic and WT forms of SHP2. To characterize these compounds, we have adopted a comprehensive biophysical and cellular testing platform. Among the inhibitors with the greatest potency and selectivity for SHP2, #02 exhibited the most substantial response in various cancer cellular models, including TNBC and AIL. In contrast to existing SHP2 allosteric inhibitors, #02 was effective in inhibiting the growth of AIL cells expressing a frequent SHP2 oncogenic variant. Moreover, we found a prolonged cellular response caused by #02 treatment, compared to the allosteric inhibitor RMC-4550, based on the compounds' inhibitory effect on ERK1/2 pathway activation. Target engagement of #02 with SHP2 was confirmed in vitro using PTS, and in cells using a cellular thermal shift assay. Using both biochemical and biophysical assessment, we could pinpoint the interaction of #02 and other furanylbenzamides with the phosphatase domain of SHP2. Selectivity of the inhibitors for SHP2 was established in vitro against two related phosphatases, and in cells using SHP2 CRISPR-Cas9 knock-out. Compared to regular MOLM-13 cells, efficacy of #01 and #02 was either completely abrogated or greatly reduced in MOLM-13-Cas9-mCherry cells in which SHP2 levels had been greatly reduced.

As described herein, a possible covalent mechanism via Michael addition to the enone double bond common to this series of compounds could be excluded, based on time-dependent, jump-dilution inhibition, inhibition and mass spectroscopic analyses. Interestingly, Michaelis-Menten kinetic experiments suggested that the novel SHP2 inhibitors do not directly compete with substrate binding at the active site. This notion was further supported by SAR studies, in which analogs of #02 with increased substrate resemblance exhibited a decrease in potency, suggesting that #02 and related analogs likely do not bind to the phosphate binding loop in the catalytic pocket.

### Compounds

Disclosed herein are furanylbenzamides which can inhibit both WT and oncogenic SHP2. Inhibitors disclosed herein may readily cross cell membranes, bind and inhibit SHP2 under physiological conditions, and effectively decrease the growth of cancer cells, including triple-negative breast cancer (TNBC) cells, acute myeloid leukemia (AML) cells expressing either WT or oncogenic SHP2, as well as patient-derived AML cells. Protein tyrosine phosphorylation is a reversible posttranslational modification that may control and fine-tune cellular responses to a wide variety of extra- and intracellular stimuli. Dysregulation of the delicate balance between phosphorylation and dephosphorylation of signaling molecules, mediated by protein tyrosine kinases (PTKs) and protein tyrosine phosphatases (PTPs), respectively, is a distinctive feature of many cancers. Thus, chemical agents to restore this balance are useful in anti-cancer efforts.

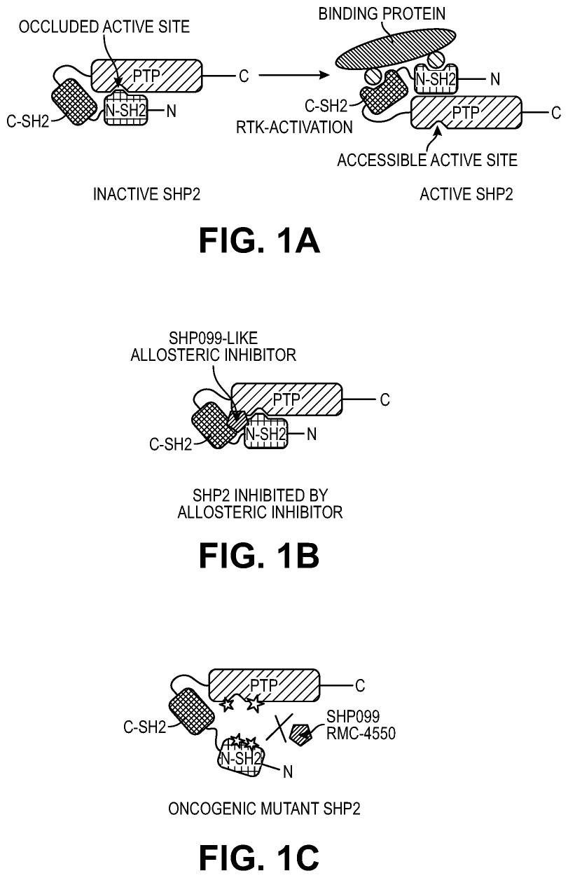

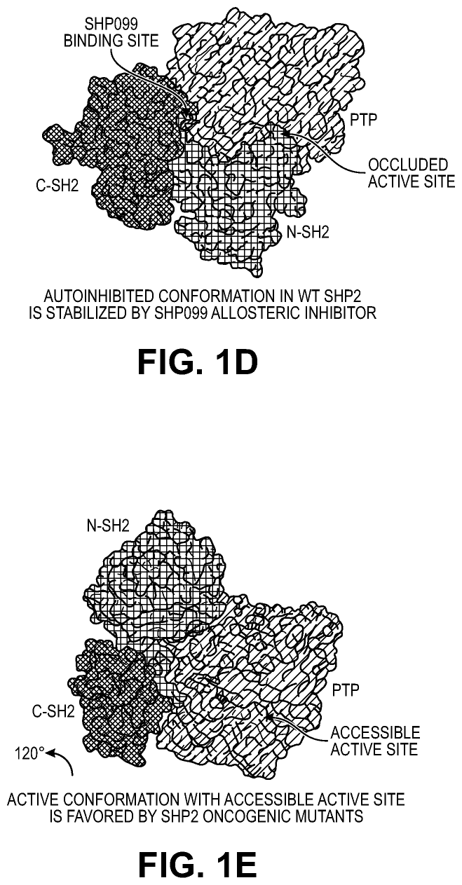

Members of the PTP enzyme family, catalyzing the reciprocal reaction, are promising drug targets for cancer therapy. A particularly prime target is the Src-homology 2 (SH2) domain-containing tyrosine phosphatase 2 (SHP2), a crucial positive regulator of receptor tyrosine kinase (RTK)-driven signaling in response to growth factors and cytokines, including signaling through the Ras/RAF/ERK, the PI3K/Akt, and the JAK/STAT pathways. SHP2 activity is tightly regulated in normal cells (FIG. 1A,D). Under resting conditions, SHP2 adopts a ‘closed’, autoinhibited conformation, in which the N-terminal SH2 domain (N—SH2) blocks access to the active site in the phosphatase domain. Upon RTK activation, SHP2 is recruited by tyrosine phosphorylated motifs within either RTK cytoplasmatic regions or adapter and scaffolding proteins via its two SH2 domains, resulting in a conformational switch that activates SHP2 by rendering the active site accessible to its substrates (‘open’, active conformation). Hyperactive SHP2 is associated with tumorigenesis, tumor maintenance, metastasis, and survival, as well as intrinsic and acquired resistance to targeted cancer drugs. FIG. 1 shows A) Model of SHP2 activation. Under resting conditions, SHP2 adopts a ‘closed’, autoinhibited conformation, in which the N-terminal SH2 domain (N—SH2) blocks access to the active site in the phosphatase (PTP) domain. Upon receptor tyrosine kinase (RTK) activation, SHP2 is recruited by tyrosine phosphorylated motifs within either RTK cytoplasmatic regions or adapter and scaffolding proteins (‘binding protein’) via its two SH2 domains, resulting in a conformational switch that activates SHP2 by rendering the active site accessible to its substrates (‘open’ conformation). B) Model of SHP2 inhibition by allosteric inhibitors such as SHP099. This ‘molecular glue’-type compounds stabilize the SHP2 inactive closed conformation. C) Model of the common mechanism of SHP2 oncogenic variants activation. A single amino acid mutation at the N—SH2/PTP domain interface prevents the intramolecular binding of the two domains, resulting in a constitutively active SHP2. Allosteric inhibitors such as SHP099 and RMC-4550 are not able to bind the open conformation preferred by the SHP2 gain-of-function mutants. D) Crystal structure of wild-type SHP2 representing the closed, autoinhibited conformation as described in A) (PDB ID: 5EHR). N—SH2 domain, blue; C—SH2 domain, green; PTP domain, orange; the allosteric inhibitor SHP099 (magenta, stick representation) binds to a channel formed by the PTP, N—SH2, and C—SH2 domains and stabilizes the inactive conformation as described in B). E) Crystal structure of the SHP2 E76K gain-of-function mutant in the open, active conformation (PDB ID: 6CRF). To adopt this conformation, the C—SH2 domain rotates by ˜120° and thereby translocates the N—SH2 domain away from the active site. SHP099-like allosteric inhibitors cannot effectively bind this active conformation as described in C).

Germline gain-of-function mutations in SHP2 that destabilize its autoinhibited conformation are observed in ˜50% of cases of Noonan syndrome, a developmental disorder with increased risk of malignancy. Numerous somatic gain-of-function mutations that similarly cause a constitutive activation of SHP2 are primarily found in leukemias. In solid tumors, SHP2 activity is often enhanced via amplification or overexpression of growth factors, RTKs, or scaffolding adapters. Cancers driven by certain Ras mutations such as KRas-G12C also depend on SHP2 activity, which promotes GDP/GTP cycling. SHP2 is also important for immune checkpoint function through modulation of PD-1, CTLA-1, and BTLA signaling, suggesting a potential immunotherapy based on targeting SHP2. Together, these data demonstrate a clear link between SHP2 signaling and cancer, confirming that SHP2 is a key target for drug discovery and development.

Targeting tyrosine phosphatases with small molecule inhibitors has been a challenge historically, because the active site of PTPs is both highly conserved and highly charged. Inhibitors that bind to the active site are often exhibit poor selectivity and limited cell membrane permeability, leading many to characterize these enzymes as undruggable. Early efforts to therapeutically target SHP2 focused on inhibitors that bind in the active site, and failed to yield compounds that combine potency and selectivity for SHP2 with efficacy in cellular models. The first truly selective SHP2 small molecule inhibitor with good cellular and in vivo efficacy, SHP099, was reported by Novartis (Allosteric inhibition of SHP2 phosphatase inhibits cancers driven by receptor tyrosine kinases. Nature 535, 148-152, (2016)). Since then, a number of SHP099-like inhibitors have been reported, and several compounds in this class are currently in clinical trials for the treatment of solid tumors with elevated RTK signaling and certain K-Ras mutations. These compounds all share a common allosteric mechanism by which they stabilize the autoinhibited conformation of SHP2 and thereby prevent recruitment and activation of the phosphatase. Specifically, the SHP099-like inhibitors act as a “molecular glue” by binding to a channel that is formed by the SHP2 phosphatase (or PTP) domain and its two SH2 domains, thereby locking SHP2 in the inactive conformation (FIG. 1B). Because this binding channel is only present in the inactive state, the SHP099-like compounds exhibit potencies that are reduced by several orders of magnitude toward many of the frequently occurring gain-of-function mutants, in which a single point mutation disturbs the SHP2 autoinhibited conformation, resulting in constitutive activation (FIG. 1C,E). Moreover, a recent report from Novartis demonstrated that fibroblast growth factor receptor (FGFR)-driven cancers, such as many breast or prostate cancers, are inherently resistant to SHP099-like inhibitors, because rapid feedback activation of the FGF receptor causes increased recruitment and activation of SHP2.

Herein described are next generation SHP2 inhibitors that are effective against cancers driven either by SHP2 oncogenic mutants, or aberrant signaling through the FGF receptor. Herein described is a series of furanylbenzamide-based inhibitors of both wild-type (WT) and oncogenic mutant SHP2. These compounds readily cross cell membranes and bind and inhibit SHP2 under physiological conditions. Inhibitor compositions disclosed herein, reduce growth of various cancer cells, including patient-derived leukemia cells, at low micromolar concentrations.

Herein described are compositions which inhibit SHP2. In some embodiments, they inhibit wild-type SHP2. In some embodiments they inhibity oncogenic SHP2. Non-limiting example compounds are detailed in Table 2.

TABLE 1
Proviso Compounds

(Z)-2-hydroxy-5-(5-((3-oxobenzo[b]thiophen-2(3H)-ylidene)methyl)furan-2-yl)-N-
phenylbenzamide
(Z)-2-hydroxy-5-(5-((3-oxobenzo[b]thiophen-2(3H)-ylidene)methyl)furan-2-yl)benzoic acid
(Z)-2-((5-phenylfuran-2-yl)methylene)benzo[b]thiophen-3(2H)-one
5-(5-((1,3-dioxo-1,3-dihydro-2H-inden-2-ylidene)methyl)furan-2-yl)-2-hydroxybenzoic acid
(E)-2-(5-((3-oxobenzo[b]thiophen-2(3H)-ylidene)methyl)furan-2-yl)benzonitrile
(E)-5-(5-((3-oxobenzo[b]thiophen-2(3H)-ylidene)methyl)furan-2-yl)isophthalic acid
(E)-3-methyl-4-(5-((3-oxobenzo[b]thiophen-2(3H)-ylidene)methyl)furan-2-yl)benzoic acid
(E)-4-methyl-3-(5-((3-oxobenzo[b]thiophen-2(3H)-ylidene)methyl)furan-2-yl)benzoic acid
2-[(5-Phenyl-2-furanyl)methylene]benzo[b]thiophen-3(2H)-one
2-[[5-(3,4-Dimethylphenyl)-2-furanyl]methylene]benzo[b]thiophen-3(2H)-one
2-[[5-(4-Acetylphenyl)-2-furanyl]methylene]benzo[b]thiophen-3(2H)-one
2-[[5-(1-Naphthalenyl)-2-furanyl]methylene]benzo[b]thiophen-3(2H)-one
2-[5-(4-Iodophenyl)-2-furanyl]methylene]benzo[b]thiophen-3(2H)-one
4-[5-[(3-Oxobenzo[b]thien-2(3H)-ylidene)methyl]-2-furanyl]benzoic acid
2-[[5-(4-Chlorophenyl)-2-furanyl]methylene]benzo[b]thiophen-3(2H)-one
(2Z)-2-[5-(4-Bromophenyl)-2-furanyl]methylene]benzo[b]thiophen-3(2H)-one
(2Z)-2-[[5-(4-Chlorophenyl)-2-furanyl]methylene]benzo[b]thiophen-3(2H)-one
2-[[5-(4-Fluorophenyl)-2-furanyl]methylene]benzo[b]thiophen-3(2H)-one
2-[[5-(4-Bromophenyl)-2-furanyl]methylene]benzo[b]thiophen-3(2H)-one
(2Z)-2-[[5-(3-Chlorophenyl)-2-furanyl]methylene]benzo[b]thiophen-3(2H)-one
(2E)-2-[[5-(3-Chlorophenyl)-2-furanyl]methylene]benzo[b]thiophen-3(2H)-one
2-[5-(3-Chlorophenyl)-2-furanyl]methylene]benzo[b]thiophen-3(2H)-one
(2Z)-2-[[5-(2-Chlorophenyl)-2-furanyl]methylene]benzo[b]thiophen-3(2H)-one
2-[5-(2-Chlorophenyl)-2-furanyl]methylene]benzo[b]thiophen-3(2H)-one
2-[5-(2-Bromophenyl)-2-furanyl]methylene]benzo[b]thiophen-3(2H)-one
2-[5-(2-Fluorophenyl)-2-furanyl]methylene]benzo[b]thiophen-3(2H)-one
Benzenesulfonic acid, 4-[5-[(3-oxobenzo[b]thiophen-2(3H)-ylidene)methyl]-2-furanyl]-
3-[5-[(3-Oxobenzo[b]thien-2(3H)-ylidene)methyl]-2-furanyl]benzoic acid
2-[5-[(3-Oxobenzo[b]thien-2(3H)-ylidene)methyl]-2-furanyl]benzoic acid
Methyl 4-[5-[(3-oxobenzo[b]thien-2(3H)-ylidene)methyl]-2-furanyl]benzoate
Benzenesulfonamide, 4-[5-[(3-oxobenzo[b]thien-2(3H)-ylidene)methyl]-2-furanyl]-
2-[[5-(3,5-Dichlorophenyl)-2-furanyl]methylene]benzo[b]thiophen-3(2H)-one
2-[[5-(4-Chloro-3-methylphenyl)-2-furanyl]methylene]benzo[b]thiophen-3(2H)-one
(2Z)-2-[5-(3-Chloro-4-methylphenyl)-2-furanyl]methylene]benzo[b]thiophen-3(2H)-one
2-[[5-(3-Chloro-4-methylphenyl)-2-furanyl]methylene]benzo[b]thiophen-3(2H)-one
2-[5-[3-(Trifluoromethyl)phenyl]-2-furanyl]methylene]benzo[b]thiophen-3(2H)-one
Ethyl 4-[5-[(3-oxobenzo[b]thien-2(3H)-ylidene)methyl]-2-furanyl]benzoate
Methyl 3-[5-[(3-oxobenzo[b]thien-2(3H)-ylidene)methyl]-2-furanyl]benzoate
1-Methylethyl 4-[5-[(3-oxobenzo[b]thien-2(3H)-ylidene)methyl]-2-furanyl]benzoate
2-[5-(4-Nitrophenyl)-2-furanyl]methylene]benzo[b]thiophen-3(2H)-one
2-[[5-(3-Chloro-2-methylphenyl)-2-furanyl]methylene]benzo[b]thiophen-3(2H)-one
(2Z)-2-[[5-[2-(Trifluoromethyl)phenyl]-2-furanyl]methylene]benzo[b]thiophen-3(2H)-one
Benzoic acid, 4-[5-[(3-oxobenzo[b]thiophen-2(3H)-ylidene)methyl]-2-furanyl]-, propyl ester
Benzoic acid, 2-[5-[(3-oxobenzo[b]thiophen-2(3H)-ylidene)methyl]-2-furanyl]-, methyl ester
2-[[5-[2-(Trifluoromethyl)phenyl]-2-furanyl]methylene]benzo[b]thiophen-3(2H)-one
Benzoic acid, 4-[5-[(3-oxobenzo[b]thiophen-2(3H)-ylidene)methyl]-2-furanyl]-, 2-propen-1-yl
ester
Benzoic acid, 4-[5-[(3-oxobenzo[b]thiophen-2(3H)-ylidene)methyl]-2-furanyl]-, 2-
methylpropyl ester
2-Hydroxy-5-[5-[(3-oxobenzo[b]thien-2(3H)-ylidene)methyl]-2-furanyl]benzoic acid
2-[5-(3-Nitrophenyl)-2-furanyl]methylene]benzo[b]thiophen-3(2H)-one
2-[5-(3,4-Dichlorophenyl)-2-furanyl]methylene]benzo[b]thiophen-3(2H)-one
Benzoic acid, 4-[5-[(3-oxobenzo[b]thiophen-2(3H)-ylidene)methyl]-2-furanyl]-, phenylmethyl
ester
Ethyl 3-[5-[(3-oxobenzo[b]thien-2(3H)-ylidene)methyl]-2-furanyl]benzoate
2-[[5-(2,3-Dichlorophenyl)-2-furanyl]methylene]benzo[b]thiophen-3(2H)-one
Benzoic acid, 3-[5-[(3-oxobenzo[b]thiophen-2(3H)-ylidene)methyl]-2-furanyl]-, 1-methylethyl
ester
Benzenesulfonamide, N,N-dimethyl-4-[5-[(3-oxobenzo[b]thien-2(3H)-ylidene)methyl]-2-
furanyl]-
2-[[5-(2,4-Dichlorophenyl)-2-furanyl]methylene]benzo[b]thiophen-3(2H)-one
2-[[5-(2,4-Difluorophenyl)-2-furanyl]methylene]benzo[b]thiophen-3(2H)-one
Benzoic acid, 2-[5-[(3-oxobenzo[b]thiophen-2(3H)-ylidene)methyl]-2-furanyl]-, ethyl ester
2-[5-(2,5-Dichlorophenyl)-2-furanyl]methylene]benzo[b]thiophen-3(2H)-one
2-[[5-(2-Bromo-4,5-dimethylphenyl)-2-furanyl]methylene]benzo[b]thiophen-3(2H)-one
Propyl 3-[5-[(3-oxobenzo[b]thiophen-2(3H)-ylidene)methyl]-2-furanyl]benzoate
Benzoic acid, 3-[5-[(3-oxobenzo[b]thiophen-2(3H)-ylidene)methyl]-2-furanyl]-, 2-propen-1-yl
ester
Benzoic acid, 3-[5-[(3-oxobenzo[b]thiophen-2(3H)-ylidene)methyl]-2-furanyl]-, 2-
methylpropyl ester
Benzoic acid, 3-[5-[(3-oxobenzo[b]thiophen-2(3H)-ylidene)methyl]-2-furanyl]-, butyl ester
2-[[5-(2-Nitrophenyl)-2-furanyl]methylene]benzo[b]thiophen-3(2H)-one
Benzoic acid, 3-[5-[(3-oxobenzo[b]thiophen-2(3H)-ylidene)methyl]-2-furanyl]-, 2-propyn-1-
ylester
Benzoic acid, 4-[5-[(3-oxobenzo[b]thiophen-2(3H)-ylidene)methyl]-2-furanyl]-,
cyanomethylester
2-[5-(3-Chloro-4-methoxyphenyl)-2-furanyl]methylene]benzo[b]thiophen-3(2H)-one
2-[[5-[4-(2-Phenyldiazenyl)phenyl]-2-furanyl]methylene]benzo[b]thiophen-3(2H)-one
2-[[5-(2-Methyl-4-nitrophenyl)-2-furanyl]methylene]benzo[b]thiophen-3(2H)-one
2-Chloro-4-[5-[(3-oxobenzo[b]thien-2(3H)-ylidene)methyl]-2-furanyl]benzoic acid
Benzo[b]thiophen-3(2H)-one, 2-[[5-(4-methyl-3-nitrophenyl)-2-furanyl]methylene]-
2-[[5-[4-Bromo-3-(trifluoromethyl)phenyl]-2-furanyl]methylene]benzo[b]thiophen-3(2H)-one
2-[[5-[4-Chloro-3-(trifluoromethyl)phenyl]-2-furanyl]methylene]benzo[b]thiophen-3(2H)-one
Acetamide, N-[[4-[5-[(3-oxobenzo[b]thiophen-2(3H)-ylidene)methyl]-2-
furanyl]phenyl]sulfonyl]-
2-[[5-(5-Chloro-2-methoxyphenyl)-2-furanyl]methylene]benzo[b]thiophen-3(2H)-one
2-[5-(2-Methyl-5-nitrophenyl)-2-furanyl]methylene]benzo[b]thiophen-3(2H)-one
2-Chloro-5-[5-[(3-oxobenzo[b]thien-2(3H)-ylidene)methyl]-2-furanyl]benzoic acid
Benzoic acid, 3-[5-[(3-oxobenzo[b]thiophen-2(3H)-ylidene)methyl]-2-furanyl]-,
cyclohexylester
5-Bromo-2-[5-[(3-oxobenzo[b]thien-2(3H)-ylidene)methyl]-2-furanyl]benzoic acid
5-Chloro-2-[5-[(3-oxobenzo[b]thien-2(3H)-ylidene)methyl]-2-furanyl]benzoic acid
2-[[5-(4-Methyl-2-nitrophenyl)-2-furanyl]methylene]benzo[b]thiophen-3(2H)-one
(2Z)-2-[5-[2-Chloro-5-(trifluoromethyl)phenyl]-2-furanyl]methylene]benzo[b]thiophen-
3(2H)-one
4-Chloro-3-[5-[(Z)-(3-oxobenzo[b]thien-2(3H)-ylidene)methyl]-2-furanyl]benzoic acid
4-Chloro-3-[5-[(E)-(3-oxobenzo[b]thien-2(3H)-ylidene)methyl]-2-furanyl]benzoic acid
2-[[5-[2-Chloro-5-(trifluoromethyl)phenyl]-2-furanyl]methylene]benzo[b]thiophen-3(2H)-one
1(3H)-Isobenzofuranone, 6-[5-[(3-oxobenzo[b]thiophen-2(3H)-ylidene)methyl]-2-furanyl]-
5-[5-[(3-Oxobenzo[b]thien-2(3H)-ylidene)methyl]-2-furanyl]-1 (3H)-isobenzofuranone
2-[5-(2-Hydroxy-5-nitrophenyl)-2-furanyl]methylene]benzo[b]thiophen-3(2H)-one
2-[[5-(2,5-Dimethyl-4-nitrophenyl)-2-furanyl]methylene]benzo[b]thiophen-3(2H)-one
9,10-Anthracenedione, 2-[5-[(3-oxobenzo[b]thiophen-2(3H)-ylidene)methyl]-2-furanyl]-
2-[[5-(3,4-Dimethyl-5-nitrophenyl)-2-furanyl]methylene]benzo[b]thiophen-3(2H)-one
2-[[5-(2,4,5-Trichlorophenyl)-2-furanyl]methylene]benzo[b]thiophen-3(2H)-one
2-[[5-(2,3-Dimethyl-4-nitrophenyl)-2-furanyl]methylene]benzo[b ]thiophen-3(2H)-one
Benzoic acid, 4-[5-[(3-oxobenzo[b]thiophen-2(3H)-ylidene)methyl]-2-furanyl]-, (2-
chlorophenyl)methyl ester
Benzoic acid, 3-[5-[(3-oxobenzo[b]thiophen-2(3H)-ylidene)methyl]-2-furanyl]-, 2-methoxy-2-
oxoethyl ester
2-[5-(2,4-Dimethyl-5-nitrophenyl)-2-furanyl]methylene]benzo[b]thiophen-3(2H)-one
2-[[5-(4,5-Dimethyl-2-nitrophenyl)-2-furanyl]methylene]benzo[b]thiophen-3(2H)-one
2-[[5-(2,5-Dimethyl-3-nitrophenyl)-2-furanyl]methylene]benzo[b]thiophen-3(2H)-one
Methyl 2-chloro-5-[5-[(3-oxobenzo[b]thien-2(3H)-ylidene)methyl]-2-furanyl]benzoate
Benzoic acid, 3-[5-[(3-oxobenzo[b]thiophen-2(3H)-ylidene)methyl]-2-furanyl]-, 2-ethoxy-2-
oxoethyl ester
Benzo[b]thiophen-3(2H)-one, 2-[[5-(5-hydroxy-2-methyl-4-nitrophenyl)-2-
furanyl]methylene]-
2-[[5-(2-Bromo-4-nitrophenyl)-2-furanyl]methylene]benzo[b]thiophen-3(2H)-one
2-[[5-(2-Chloro-4-nitrophenyl)-2-furanyl]methylene]benzo[b]thiophen-3(2H)-one
2-[[5-(2-Methoxy-4-nitrophenyl)-2-furanyl]methylene]benzo[b]thiophen-3(2H)-one
4-[5-[(3-Oxobenzo[b ]thien-2(3H)-ylidene)methyl]-2-furanyl]-N-2-
thiazolylbenzenesulfonamide
2-[[5-(2-Methoxy-5-nitrophenyl)-2-furanyl]methylene]benzo[b]thiophen-3(2H)-one
2-[[5-(4-Chloro-3-nitrophenyl)-2-furanyl]methylene]benzo[b]thiophen-3(2H)-one
2-[[5-(2-Chloro-5-nitrophenyl)-2-furanyl]methylene]benzo[b]thiophen-3(2H)-one
2-[[5-(4-Methoxy-2-nitrophenyl)-2-furanyl]methylene]benzo[b]thiophen-3(2H)-one
Benzoic acid, 2-chloro-5-[5-[(Z)-(3-oxobenzo[b]thiophen-2(3H)-ylidene)methyl]-2-furanyl]-
ethyl ester
Ethyl 2-chloro-5-[5-[(3-oxobenzo[b]thien-2(3H)-ylidene)methyl]-2-furanyl]benzoate
2-[[5-(4-Ethoxy-3-nitrophenyl)-2-furanyl]methylene]benzo[b]thiophen-3(2H)-one
Benzoic acid, 5-bromo-2-[5-[(3-oxobenzo[b]thiophen-2(3H)-ylidene)methyl]-2-furanyl]-, ethyl
ester
2-[[5-(4-Chloro-2-nitrophenyl)-2-furanyl]methylene]benzo[b]thiophen-3(2H)-one
2-[[5-(2,6-Dibromo-4-nitrophenyl)-2-furanyl]methylene]benzo[b]thiophen-3(2H)-one
Benzoic acid, 2-chloro-5-[5-[(Z)-(3-oxobenzo[b]thiophen-2(3H)-ylidene)methyl]-2-furanyl]-,
butyl ester
Benzoic acid, 2-chloro-5-[5-[(3-oxobenzo[b]thiophen-2(3H)-ylidene)methyl]-2-furanyl]-, 2-
propyn-1-yl ester
Butyl 2-chloro-5-[5-[(3-oxobenzo[b]thien-2(3H)-ylidene)methyl]-2-furanyl]benzoate
Benzenesulfonamide, N-(5-ethyl-1,3,4-thiadiazol-2-yl)-4-[5-[(3-oxobenzo[b]thiophen-2(3H)-
ylidene)methyl]-2-furanyl]-
Phenylmethyl 2-chloro-5-[5-[(3-oxobenzo[b]thien-2(3H)-ylidene)methyl]-2-furanyl]benzoate
2-[[5-(5-Chloro-4-methyl-2-nitrophenyl)-2-furanyl]methylene]benzo[b]thiophen-3(2H)-one
Ethyl 3-nitro-4-[5-[(3-oxobenzo[b]thien-2(3H)-ylidene)methyl]-2-furanyl]benzoate
Benzoic acid, 2-chloro-5-[5-[(3-oxobenzo[b]thiophen-2(3H)-ylidene)methyl]-2-furanyl]-,
cyclohexyl ester
2-[[5-[3-Nitro-4-(1-piperidinyl)phenyl]-2-furanyl]methylene]benzo[b]thiophen-3(2H)-one
5-[5-[(3-Oxobenzo[b ]thien-2(3H)-ylidene)methyl]-2-furanyl]-1 H-isoindole-1,3(2H)-dione
2-[[5-[4-(4-Morpholinyl)-3-nitrophenyl]-2-furanyl]methylene]benzo[b]thiophen-3(2H)-one
2-[[5-[2-(4-Morpholinyl)-5-nitrophenyl]-2-furanyl]methylene]benzo[b]thiophen-3(2H)-one
2-[[5-(4-Bromophenyl)-2-furanyl]methylene]-5-methyl-3(2H)-benzofuranone
2-[[5-(4-Chlorophenyl)-2-furanyl]methylene]-5-methyl-3(2H)-benzofuranone
2-[[5-(4-Fluorophenyl)-2-furanyl]methylene]-5-methyl-3(2H)-benzofuranone
2-[[5-(3-Chlorophenyl)-2-furanyl]methylene]-5-methyl-3(2H)-benzofuranone
2-[[5-(3-Bromophenyl)-2-furanyl]methylene]-5-methyl-3(2H)-benzofuranone
2-[5-(4-Bromophenyl)-2-furanyl]methylene]-5,6-dimethyl-3(2H)-benzofuranone
2-[[5-(4-Chlorophenyl)-2-furanyl]methylene]-5,6-dimethyl-3(2H)-benzofuranone
2-[[5-(4-Fluorophenyl)-2-furanyl]methylene]-5,6-dimethyl-3(2H)-benzofuranone
5-Bromo-2-[[5-(3-bromophenyl)-2-furanyl]methylene]-3(2H)-benzofuranone
5-Chloro-2-[[5-(3-chlorophenyl)-2-furanyl]methylene]-3(2H)-benzofuranone
2-[[5-(2-Chlorophenyl)-2-furanyl]methylene]-5-methyl-3(2H)-benzofuranone
5-Bromo-2-[5-(4-bromophenyl)-2-furanyl]methylene]-3(2H)-benzofuranone
5-Chloro-2-[5-(4-chlorophenyl)-2-furanyl]methylene]-3(2H)-benzofuranone
2-[5-(3-Chlorophenyl)-2-furanyl]methylene]-5,6-dimethyl-3(2H)-benzofuranone
2-[[5-(3-Bromophenyl)-2-furanyl]methylene]-5,6-dimethyl-3(2H)-benzofuranone
5-Bromo-2-[[5-(3-chlorophenyl)-2-furanyl]methylene]-3(2H)-benzofuranone
2-[[5-(3-Bromophenyl)-2-furanyl]methylene]-5-chloro-3(2H)-benzofuranone
5-Methyl-2-[[5-[3-(trifluoromethyl)phenyl]-2-furanyl]methylene]-3(2H)-benzofuranone
5-Bromo-2-[5-(4-chlorophenyl)-2-furanyl]methylene]-3(2H)-benzofuranone
5-Bromo-2-[[5-(4-fluorophenyl)-2-furanyl]methylene]-3(2H)-benzofuranone
2-[[5-(4-Bromophenyl)-2-furanyl]methylene]-5-chloro-3(2H)-benzofuranone
5-Chloro-2-[5-(4-fluorophenyl)-2-furanyl]methylene]-3(2H)-benzofuranone
2-[[5-(2-Chlorophenyl)-2-furanyl]methylene]-5,6-dimethyl-3(2H)-benzofuranone
5-Chloro-2-[[5-(2-chlorophenyl)-2-furanyl]methylene]-3(2H)-benzofuranone
2-[[5-(3,4-Dichlorophenyl)-2-furanyl]methylene]-5-methyl-3(2H)-benzofuranone
5,6-Dimethyl-2-[[5-[3-(trifluoromethyl)phenyl]-2-furanyl]methylene]-3(2H)-benzofuranone
5-Bromo-2-[5-(2-chlorophenyl)-2-furanyl]methylene]-3(2H)-benzofuranone
5-Chloro-2-[5-(3,4-dichlorophenyl)-2-furanyl]methylene]-3(2H)-benzofuranone
5-Bromo-2-[[5-(3,4-dichlorophenyl)-2-furanyl]methylene]-3(2H)-benzofuranone
5-Methyl-2-[[5-(3-nitrophenyl)-2-furanyl]methylene]-3(2H)-benzofuranone
2-[[5-(2,5-Dichlorophenyl)-2-furanyl]methylene]-5-methyl-3(2H)-benzofuranone
2-[[5-(3,4-Dichlorophenyl)-2-furanyl]methylene]-5,6-dimethyl-3(2H)-benzofuranone
5-Bromo-2-[[5-[3-(trifluoromethyl)phenyl]-2-furanyl]methylene]-3(2H)-benzofuranone
5-Chloro-2-[[5-[3-(trifluoromethyl)phenyl]-2-furanyl]methylene]-3(2H)-benzofuranone
2-[5-(3-Chlorophenyl)-2-furanyl]methylene]-6,7-dihydroxy-3(2H)-benzofuranone
5-Chloro-2-[5-(2,5-dichlorophenyl)-2-furanyl]methylene]-3(2H)-benzofuranone
5-Bromo-2-[5-(2,5-dichlorophenyl)-2-furanyl]methylene]-3(2H)-benzofuranone
Methyl 2-[5-(4-fluorophenyl)-2-furanyl]methylene]-2,3-dihydro-3-oxo-5-
benzofurancarboxylate
Methyl 2-[[5-(4-bromophenyl)-2-furanyl]methylene]-2,3-dihydro-3-oxo-5-
benzofurancarboxylate
Methyl 2-[[5-(4-chlorophenyl)-2-furanyl]methylene]-2,3-dihydro-3-oxo-5-
benzofurancarboxylate
2-[[5-(2-Chlorophenyl)-2-furanyl]methylene]-6,7-dihydroxy-3(2H)-benzofuranone
5,6-Dimethyl-2-[5-(3-nitrophenyl)-2-furanyl]methylene]-3(2H)-benzofuranone
2-[[5-(2,5-Dichlorophenyl)-2-furanyl]methylene]-5,6-dimethyl-3(2H)-benzofuranone
Methyl 2-[[5-(3-chlorophenyl)-2-furanyl]methylene]-2,3-dihydro-3-oxo-5-
benzofurancarboxylate
Methyl 2-[[5-(3-bromophenyl)-2-furanyl]methylene]-2,3-dihydro-3-oxo-5-
benzofurancarboxylate
5-Methyl-2-[[5-(2-nitrophenyl)-2-furanyl]methylene]-3(2H)-benzofuranone
6,7-Dihydroxy-2-[5-[3-(trifluoromethyl)phenyl]-2-furanyl]methylene]-3(2H)-benzofuranone
Methyl 2-[[5-(2-chlorophenyl)-2-furanyl]methylene]-2,3-dihydro-3-oxo-5-
benzofurancarboxylate
Methyl 2,3-dihydro-3-oxo-2-[[5-[3-(trifluoromethyl)phenyl]-2-furanyl]methylene]-5-
benzofurancarboxylate
5,6-Dimethyl-2-[[5-(2-nitrophenyl)-2-furanyl]methylene]-3(2H)-benzofuranone
6,7-Dihydroxy-2-[5-[2-(trifluoromethyl)phenyl]-2-furanyl]methylene]-3(2H)-benzofuranone
5-Bromo-2-[[5-(3-nitrophenyl)-2-furanyl]methylene]-3(2H)-benzofuranone
5-Chloro-2-[[5-(3-nitrophenyl)-2-furanyl]methylene]-3(2H)-benzofuranone
Methyl 2-[[5-(3,4-dichlorophenyl)-2-furanyl]methylene]-2,3-dihydro-3-oxo-5-
benzofurancarboxylate
Methyl 2,3-dihydro-2-[[5-(3-nitrophenyl)-2-furanyl]methylene]-3-oxo-5-
benzofurancarboxylate
(2Z)-4,6-Dihydroxy-5-(2-propen-1-yl)-2-[[5-[2-(trifluoromethyl)phenyl]-2-
furanyl]methylene]-3(2H)-benzofuranone
Methyl 2-[[5-(2,5-dichlorophenyl)-2-furanyl]methylene]-2,3-dihydro-3-oxo-5-
benzofurancarboxylate
5-Bromo-2-[[5-(2-nitrophenyl)-2-furanyl]methylene]-3 (2H)-benzofuranone
5-Chloro-2-[[5-(2-nitrophenyl)-2-furanyl]methylene]-3 (2H)-benzofuranone
Methyl 2,3-dihydro-2-[[5-(2-nitrophenyl)-2-furanyl]methylene]-3-oxo-5-
benzofurancarboxylate
5-(5-Formyl-2-furanyl)-2-hydroxy-N-phenylbenzamide
5-(5-Formyl-2-furanyl)-2-hydroxy-N-(4-methylphenyl)benzamide
N-(4-Ethylphenyl)-5-(5-formyl-2-furanyl)-2-hydroxybenzamide
3-[[5-(2-Furanyl)-2-hydroxybenzoyl]amino][1,1'-biphenyl]-4-carboxylic acid
5-(5-Formyl-2-furanyl)-2-hydroxy-N-(4-methoxyphenyl)benzamide
N-(3-Chloro-4-methylphenyl)-5-(5-formyl-2-furanyl)-2-hydroxybenzamide
2-Hydroxy-5-[5-[[2-(methylsulfonyl)ethyl]amino]methyl]-2-furanyl]-N-phenylbenzamide
N-(3-Chloro-4-methoxyphenyl)-5-(5-formyl-2-furanyl)-2-hydroxybenzamide
2-Hydroxy-N-(4-methylphenyl)-5-[5-[[[2-(methylsulfonyl)ethyl]amino]methyl]-2-
furanyl]benzamide
N-(4-Ethylphenyl)-2-hydroxy-5-[5-[[[2-(methylsulfonyl)ethyl]amino]methyl]-2-
furanyl]benzamide
N-[4-[(3-Fluorophenyl)methoxy]phenyl]-5-(5-formyl-2-furanyl)-2-hydroxybenzamide
2-Hydroxy-N-(4-methoxyphenyl)-5-[5-[[[2-(methylsulfonyl)ethyl]amino]methyl]-2-
furanyl]benzamide
N-[4-[(3-Fluorophenyl)methoxy]-3-methylphenyl]-5-(5-formyl-2-furanyl)-2-
hydroxybenzamide
Methyl 3-[[2-(acetyloxy)-5-(2-furanyl)benzoyl]amino][1,1'-biphenyl]-4-carboxylate
N-(3-Chloro-4-methylphenyl)-2-hydroxy-5-[5-[[[2-(methylsulfonyl)ethyl]amino]methyl]-2-
furanyl]benzamide
5-(2-Benzofuranyl)-2-hydroxy-N-[3-[2-(2-methoxyphenyl)ethoxy ]phenyl]benzamide
7-Quinolinecarboxamide, N-(4-cyanophenyl)-5-(2-furanyl)-8-hydroxy-
N-[3-Chloro-4-[(3-fluorophenyl)methoxy]phenyl]-5-(5-formyl-2-furanyl)-2-
hydroxybenzamide
N-[4-[(3-Fluorophenyl)methoxy]-3-methoxyphenyl]-5-(5-formyl-2-furanyl)-2-
hydroxybenzamide
N-(3-Chloro-4-methoxyphenyl)-2-hydroxy-5-[5-[[2-(methylsulfonyl)ethyl]amino]methyl]-2-
furanyl]benzamide
N-[4-[(3-Fluorophenyl)methoxy]phenyl]-2-hydroxy-5-[5-[[[2-
(methylsulfonyl)ethyl]amino]methyl]-2-furanyl]benzamide
N-[4-[(3-Fluorophenyl)methoxy]-3-methylphenyl]-2-hydroxy-5-[5-[[2-
(methylsulfonyl)ethyl]amino]methyl]-2-furanyl]benzamide
N-[3-Chloro-4-(2-pyridinylmethoxy)phenyl]-5-(5-formyl-2-furanyl)-2-hydroxybenzamide
N-[1-[(3-Fluorophenyl)methyl]-1H-indazol-5-yl]-5-(5-formyl-2-furanyl)-2-hydroxybenzamide
N-[3-Chloro-4-[(3-fluorophenyl)methoxy ]phenyl]-2-hydroxy-5-[5-[2-
(methylsulfonyl)ethyl]amino]methyl]-2-furanyl]benzamide
N-[4-[(3-Fluorophenyl)methoxy]-3-methoxyphenyl]-2-hydroxy-5-[5-[2-
(methylsulfonyl)ethyl]amino]methyl]-2-furanyl]benzamide
N-[3-Chloro-4-(2-pyridinylmethoxy)phenyl]-2-hydroxy-5-[5-[[2-
(methylsulfonyl)ethyl]amino]methyl]-2-furanyl]benzamide
N-[1-[(3-Fluorophenyl)methyl]-1H-indazol-5-yl]-2-hydroxy-5-[5-[2-
(methylsulfonyl)ethyl]amino]methyl]-2-furanyl]benzamide
2-[(5-Phenyl-2-furanyl)methylene]-1H-indene-1,3(2H)-dione
2-[[5-(4-Methylphenyl)-2-furanyl]methylene]-1H-indene-1,3(2H)-dione
4-[5-[(1,3-Dihydro-1-oxo-2H-inden-2-ylidene)methyl]-2-furanyl]benzoic acid
2-[5-[(1,3-Dihydro-1-oxo-2H-inden-2-ylidene)methyl]-2-furanyl]benzoic acid
2-[[5-(4-Chlorophenyl)-2-furanyl]methylene]-2,3-dihydro-1H-inden-1-one
2-[[5-(4-Bromophenyl)-2-furanyl]methylene]-2,3-dihydro-1H-inden-1-one
2-[[5-(4-Fluorophenyl)-2-furanyl]methylene]-2,3-dihydro-1H-inden-1-one
2-[[5-(3-Chlorophenyl)-2-furanyl]methylene]-2,3-dihydro-1H-inden-1-one
3-[5-[(1,3-Dihydro-1-oxo-2H-inden-2-ylidene)methyl]-2-furanyl]benzoic acid
2-[[5-(4-Methoxyphenyl)-2-furanyl]methylene]-1H-indene-1,3(2H)-dione
2-[5-[(1,3-Dihydro-1-oxo-2H-inden-2-ylidene)methyl]-2-furanyl]benzonitrile
(2E)-2-[[5-(2-Fluorophenyl)-2-furanyl]methylene]-2,3-dihydro-1H-inden-1-one
(2E)-2-[[5-(2-Bromophenyl)-2-furanyl]methylene]-2,3-dihydro-1H-inden-1-one
Benzonitrile, 4-[5-[(1,3-dihydro-1,3-dioxo-2H-inden-2-ylidene)methyl]-2-furanyl]-
Methyl 4-[5-[(1,3-dihydro-1-oxo-2H-inden-2-ylidene)methyl]-2-furanyl]benzoate
4-[5-[(1,3-Dihydro-1,3-dioxo-2H-inden-2-ylidene)methyl]-2-furanyl]benzoic acid
2-[5-[(1,3-Dihydro-1,3-dioxo-2H-inden-2-ylidene)methyl]-2-furanyl]benzoic acid
3-[5-[(1,3-Dihydro-1,3-dioxo-2H-inden-2-ylidene)methyl]-2-furanyl]benzoic acid
2-[[5-(4-Aminophenyl)-2-furanyl]methylene]-1H-indene-1,3(2H)-dione
1H-Indene-1,3(2H)-dione, 2-[5-(4-iodophenyl)-2-furanyl]methylene]-
2-[[5-(4-Chlorophenyl)-2-furanyl]methylene]-1H-indene-1,3(2H)-dione
2-[[5-(4-Bromophenyl)-2-furanyl]methylene]-1H-indene-1,3(2H)-dione
2-[[5-(4-Fluorophenyl)-2-furanyl]methylene]-1H-indene-1,3(2H)-dione
2-[[5-(3-Chlorophenyl)-2-furanyl]methylene]-1H-indene-1,3(2H)-dione
2-[[5-(3-Bromophenyl)-2-furanyl]methylene]-1H-indene-1,3(2H)-dione
2-[[5-(3-Fluorophenyl)-2-furanyl]methylene]-1H-indene-1,3(2H)-dione
Methyl 2-[5-[(1,3-dihydro-1-oxo-2H-inden-2-ylidene)methyl]-2-furanyl]benzoate
2,3-Dihydro-2-[[5-[3-(trifluoromethyl)phenyl]-2-furanyl]methylene]-1H-inden-1-one
Methyl 3-[5-[(1,3-dihydro-1-oxo-2H-inden-2-ylidene)methyl]-2-furanyl]benzoate
Ethyl 4-[5-[(1,3-dihydro-1-oxo-2H-inden-2-ylidene)methyl]-2-furanyl]benzoate
2-[5-[(1,3-Dihydro-1,3-dioxo-2H-inden-2-ylidene)methyl]-2-furanyl]benzonitrile
2-[[5-(3-Bromo-4-methylphenyl)-2-furanyl]methylene]-2,3-dihydro-1H-inden-1-one
2-[[5-(3-Chloro-4-methylphenyl)-2-furanyl]methylene]-2,3-dihydro-1H-inden-1-one
Methyl 4-[5-[(1,3-dihydro-1,3-dioxo-2H-inden-2-ylidene)methyl]-2-furanyl]benzoate
2-[[5-(2-Chlorophenyl)-2-furanyl]methylene]-1H-indene-1,3(2H)-dione
2-[[5-(2-Fluorophenyl)-2-furanyl]methylene]-1H-indene-1,3(2H)-dione
2,3-Dihydro-2-[5-(4-nitrophenyl)-2-furanyl]methylene]-1H-inden-1-one
4-[5-[(1,3-Dihydro-1,3-dioxo-2H-inden-2-ylidene)methyl]-2-furanyl]-3-methylbenzoic acid
3-[5-[(1,3-Dihydro-1,3-dioxo-2H-inden-2-ylidene)methyl]-2-furanyl]-4-methylbenzoicacid
Methyl 2-[5-[(1,3-dihydro-1,3-dioxo-2H-inden-2-ylidene)methyl]-2-furanyl]benzoate
5-[5-[(1,3-Dihydro-1,3-dioxo-2H-inden-2-ylidene)methyl]-2-furanyl]-2-methylbenzoicacid
2-[[5-(4-Fluorophenyl)-2-furanyl]methylene]-2,3-dihydro-4-hydroxy-1H-inden-1-one
Ethyl 3-[5-[(1,3-dihydro-1-oxo-2H-inden-2-ylidene)methyl]-2-furanyl]benzoate
Methyl 3-[5-[(1,3-dihydro-1,3-dioxo-2H-inden-2-ylidene)methyl]-2-furanyl]benzoate
2-[[5-[3-(Trifluoromethyl)phenyl]-2-furanyl]methylene]-1H-indene-1,3(2H)-dione
2,3-Dihydro-2-[[5-(3-nitrophenyl)-2-furanyl]methylene]-1H-inden-1-one
Ethyl 4-[5-[(1,3-dihydro-1,3-dioxo-2H-inden-2-ylidene)methyl]-2-furanyl]benzoate
2-[[5-(3-Chloro-2-methylphenyl)-2-furanyl]methylene]-1H-indene-1,3(2H)-dione
1H-Indene-1,3(2H)-dione, 2-[[5-(4-bromo-3-methylphenyl)-2-furanyl]methylene]-
4-[5-[(1,3-Dihydro-1,3-dioxo-2H-inden-2-ylidene)methyl]-2-furanyl]benzenesulfonamide
2-[[5-(4-Chloro-3-methylphenyl)-2-furanyl]methylene]-1H-indene-1,3(2H)-dione
5-[5-[(1,3-Dihydro-1,3-dioxo-2H-inden-2-ylidene)methyl]-2-furanyl]-2-hydroxybenzoic acid
2-[[5-(3-Bromo-4-methylphenyl)-2-furanyl]methylene]-1H-indene-1,3(2H)-dione
2-[5-(3-Chloro-4-methylphenyl)-2-furanyl]methylene]-1H-indene-1,3(2H)-dione
2-[[5-(3,4-Dichlorophenyl)-2-furanyl]methylene]-2,3-dihydro-1H-inden-1-one
2-[[5-(5-Chloro-2-methylphenyl)-2-furanyl]methylene]-1H-indene-1,3(2H)-dione
2-[[5-[3,5-Bis(trifluoromethyl)phenyl]-2-furanyl]methylene]-2,3-dihydro-1H-inden-1-one
2-[[5-(2,3-Dichlorophenyl)-2-furanyl]methylene]-2,3-dihydro-1H-inden-1-one
Benzoic acid, 4-[5-[(1,3-dihydro-1,3-dioxo-2H-inden-2-ylidene)methyl]-2-furanyl]-3-methyl-,
methyl ester
2-[[5-(2-Bromo-4-methylphenyl)-2-furanyl]methylene]-1H-indene-1,3(2H)-dione
2-[5-(4-Nitrophenyl)-2-furanyl]methylene]-1H-indene-1,3(2H)-dione
2-[[5-(2,4-Dichlorophenyl)-2-furanyl]methylene]-2,3-dihydro-1H-inden-1-one
Ethyl 3-[5-[(1,3-dihydro-1,3-dioxo-2H-inden-2-ylidene)methyl]-2-furanyl]benzoate
2-[[5-(2,5-Dichlorophenyl)-2-furanyl]methylene]-2,3-dihydro-1H-inden-1-one
Methyl 2-[5-[(1,3-dihydro-4-hydroxy-1-oxo-2H-inden-2-ylidene)methyl]-2-furanyl]benzoate
Benzoic acid, 3-[5-[(1,3-dihydro-1,3-dioxo-2H-inden-2-ylidene)methyl]-2-furanyl]-2-methyl-,
methyl ester
2,3-Dihydro-2-[[5-(2-nitrophenyl)-2-furanyl]methylene]-1H-inden-1-one
2-[[5-(3-Nitrophenyl)-2-furanyl]methylene]-1H-indene-1,3(2H)-dione
5-Chloro-2,3-dihydro-2-[5-[3-(trifluoromethyl)phenyl]-2-furanyl]methylene]-1H-inden-1-one
2-[[5-(3,4-Dichlorophenyl)-2-furanyl]methylene]-1H-indene-1,3(2H)-dione
2-[[5-[2-(Trifluoromethoxy )phenyl]-2-furanyl]methylene]-1H-indene-1,3(2H)-dione
2,3-Dihydro-2-[5-(2-methyl-5-nitrophenyl)-2-furanyl]methylene]-1H-inden-1-one
2-[5-[(1,3-Dihydro-5,6-dimethoxy-1-oxo-2H-inden-2-ylidene)methyl]-2-furanyl]benzoic acid
2-[[5-(2,3-Dichlorophenyl)-2-furanyl]methylene]-1H-indene-1,3(2H)-dione
2-[[5-(4-Bromo-3-chlorophenyl)-2-furanyl]methylene]-1H-indene-1,3(2H)-dione
2,3-Dihydro-4-hydroxy-2-[5-(4-nitrophenyl)-2-furanyl]methylene]-1H-inden-1-one
2-[[5-(2,4-Dichlorophenyl)-2-furanyl]methylene]-1H-indene-1,3(2H)-dione
2-[[5-(2,4-Difluorophenyl)-2-furanyl]methylene]-1H-indene-1,3(2H)-dione
2-[[5-(2,5-Dichlorophenyl)-2-furanyl]methylene]-1H-indene-1,3(2H)-dione
2-[5-(2-Methyl-4-nitrophenyl)-2-furanyl]methylene]-1H-indene-1,3(2H)-dione
2,3-Dihydro-2-[5-(4-methyl-3-nitrophenyl)-2-furanyl]methylene]-1H-inden-1-one
2-Chloro-5-[5-[(1,3-dihydro-1-oxo-2H-inden-2-ylidene)methyl]-2-furanyl]benzoic acid
2-[[5-(3-Chloro-4-methoxyphenyl)-2-furanyl]methylene]-1H-indene-1,3(2H)-dione
2-[[5-[2-Chloro-4-(trifluoromethyl)phenyl]-2-furanyl]methylene]-2,3-dihydro-1H-inden-1-one
2-[[5-(2-Nitrophenyl)-2-furanyl]methylene]-1H-indene-1,3(2H)-dione
2-[[5-(2-Methyl-5-nitrophenyl)-2-furanyl]methylene]-1H-indene-1,3(2H)-dione
2,3-Dihydro-2-[5-(4-methyl-2-nitrophenyl)-2-furanyl]methylene]-1H-inden-1-one
1H-Indene-1,3(2H)-dione, 2,2'-[(9,9-dimethyl-9H-fluorene-2,7-diyl)bis(5,2-
furandiylmethylidyne)]bis-
2-Chloro-4-[5-[(1,3-dihydro-1,3-dioxo-2H-inden-2-ylidene)methyl]-2-furanyl]benzoic acid
2-Chloro-5-[5-[(1,3-dihydro-1,3-dioxo-2H-inden-2-ylidene)methyl]-2-furanyl]benzoic acid
2-[5-(4-Methyl-3-nitrophenyl)-2-furanyl]methylene]-1H-indene-1,3(2H)-dione
4-Chloro-3-[5-[(1,3-dihydro-1,3-dioxo-2H-
inden-2-ylidene)methyl]-2-furanyl]benzoic acid
2,3-Dihydro-4-hydroxy-2-[5-(2-nitrophenyl)-2-furanyl]methylene]-1H-inden-1-one
Methyl 2-chloro-5-[5-[(1,3-dihydro-1-oxo-2H-inden-2-ylidene)methyl]-2-furanyl]benzoate
2-[5-(4-Methyl-2-nitrophenyl)-2-furanyl]methylene]-1H-indene-1,3(2H)-dione
2,3-Dihydro-2-[5-[4-(4-morpholinylsulfonyl)phenyl]-2-furanyl]methylene]-1H-inden-1-one
2-[[5-(2,5-Dimethyl-4-nitrophenyl)-2-furanyl]methylene]-1 H-indene-1,3(2H)-dione
2-[[5-[2-Chloro-5-(trifluoromethyl)phenyl]-2-furanyl]methylene]-1H-indene-1,3(2H)-dione
2-[[5-(2,3-Dimethyl-4-nitrophenyl)-2-furanyl]methylene]-1H-indene-1,3(2H)-dione
2,3-Dihydro-2-[5-(2-methoxy-4-nitrophenyl)-2-furanyl]methylene]-1H-inden-1-one
2-[[5-(2-Chloro-4-nitrophenyl)-2-furanyl]methylene]-2,3-dihydro-1H-inden-1-one
2-[[5-(2-Bromo-4-nitrophenyl)-2-furanyl]methylene]-2,3-dihydro-1H-inden-1-one
2-[[5-(2,4-Dimethyl-5-nitrophenyl)-2-furanyl]methylene]-1H-indene-1,3(2H)-dione
2-[5-(3,4-Dimethyl-5-nitrophenyl)-2-furanyl]methylene]-1H-indene-1,3(2H)-dione
2-[[5-(4-Chloro-3-nitrophenyl)-2-furanyl]methylene]-2,3-dihydro-1H-inden-1-one
2-[[5-(2,5-Dimethyl-3-nitrophenyl)-2-furanyl]methylene]-1H-indene-1,3(2H)-dione
Methyl 2-chloro-5-[5-[(1,3-dihydro-1,3-dioxo-2H-inden-2-ylidene)methyl]-2-furanyl]benzoate
Ethyl 2-chloro-5-[5-[(1,3-dihydro-1-oxo-2H-inden-2-ylidene)methyl]-2-furanyl]benzoate
2-[[5-(2-Chloro-5-nitrophenyl)-2-furanyl]methylene]-2,3-dihydro-1H-inden-1-one
2-[[5-(2-Methoxy-4-nitrophenyl)-2-furanyl]methylene]-1H-indene-1,3(2H)-dione
2,3-Dihydro-2-[[5-(4-methoxy-2-nitrophenyl)-2-furanyl]methylene]-1H-inden-1-one
2-[[5-(2-Methoxy-5-nitrophenyl)-2-furanyl]methylene]-1H-indene-1,3(2H)-dione
2-[[5-(4,5-Dimethyl-2-nitrophenyl)-2-furanyl]methylene]-1H-indene-1,3(2H)-dione
2,2'-[(9,9-Dihexyl-9H-fluorene-2, 7-diyl)bis(5,2-furandiylmethylidyne)]bis[1H-indene-
1,3(2H)-dione]
2,2'-[(9,9-Dioctyl-9H-fluorene-2,7-diyl)bis(5,2-furandiylmethylidyne)]bis[1H-indene-1,3(2H)-
dione]
2-[[5-(4-Bromo-2-nitrophenyl)-2-furanyl]methylene]-2,3-dihydro-1H-inden-1-one
2-[[5-(4-Chloro-2-nitrophenyl)-2-furanyl]methylene]-2,3-dihydro-1H-inden-1-one
2-[[5-(4-Fluoro-2-nitrophenyl)-2-furanyl]methylene]-2,3-dihydro-1H-inden-1-one
2-[[5-(2-Bromo-4-nitrophenyl)-2-furanyl]methylene]-1H-indene-1,3(2H)-dione
2-[[5-(2-Chloro-4-nitrophenyl)-2-furanyl]methylene]-1H-indene-1,3(2H)-dione
Ethyl 2-chloro-5-[5-[(1,3-dihydro-1,3-dioxo-2H-inden-2-ylidene)methyl]-2-furanyl]benzoate
2-[[5-(4-Chloro-3-nitrophenyl)-2-furanyl]methylene]-1H-indene-1,3(2H)-dione
2-[[5-(4-Methoxy-2-nitrophenyl)-2-furanyl]methylene]-1H-indene-1,3(2H)-dione
1H-Indene-1,3(2H)-dione, 2-[5-(2-methoxy-3-nitrophenyl)-2-furanyl]methylene]-
5-[(1,3-Dihydro-1,3-dioxo-2H-inden-2-ylidene)phenylmethyl]-4-(2-methyl-1-oxopropyl)-2-
phenyl-3-furancarbonitrile
2-[[5-(2-Chloro-5-nitrophenyl)-2-furanyl]methylene]-1H-indene-1,3(2H)-dione
2-[[5-(4-Bromo-2-nitrophenyl)-2-furanyl]methylene]-1H-indene-1,3(2H)-dione
2-[[5-(4-Fluoro-2-nitrophenyl)-2-furanyl]methylene]-1H-indene-1,3(2H)-dione
2-[[5-(4-Chloro-2-nitrophenyl)-2-furanyl]methylene]-1H-indene-1,3(2H)-dione
2-[5-[2-Nitro-4-(trifluoromethyl)phenyl]-2-furanyl]methylene]-1H-indene-1,3(2H)-dione
2-[[5-(5-Chloro-4-methyl-2-nitrophenyl)-2-furanyl]methylene]-1H-indene-1,3(2H)-dione
Ethyl 4-[5-[(1,3-dihydro-1,3-dioxo-2H-inden-2-ylidene)methyl]-2-furanyl]-3-nitrobenzoate
1H-Indene-1,3(2H)-dione, 2-[[5-(2,4-dichloro-5-nitrophenyl)-2-furanyl]methylene]-
5-[(1,3-Dihydro-1,3-dioxo-2H-inden-2-ylidene)phenylmethyl]-4-(4-nitrobenzoyl)-2-phenyl-3-
furancarbonitrile
2-[(3-Benzoyl-4-oxo-4H-furo[3,2-c][1]benzopyran-2-yl)phenylmethylene]-1H-indene-
1,3(2H)-dione
2-[[3-(2-Methyl-1-oxopropyl)-4-oxo-4H-furo[3,2-c][1]benzopyran-2-yl]phenylmethylene]-1H-
indene-1,3(2H)-dione
2-[(3-Benzoyl-4-oxo-4H-furo[3,2-c][1]benzopyran-2-yl)(4-methylphenyl)methylene]-1H-
indene-1,3(2H)-dione
2-[[3-(4-Methoxybenzoyl)-4-oxo-4H-furo[3,2-c][1]benzopyran-2-yl]phenylmethylene]-1H-
indene-1,3(2H)-dione
2-[(3-Benzoyl-4-oxo-4H-furo[3,2-c][1]benzopyran-2-yl)(4-methoxyphenyl)methylene]-1H-
indene-1,3(2H)-dione
2-[[3-(4-Methoxybenzoyl)-4-ox0-4H-furo[3,2-c][1]benzopyran-2-yl](4-
methoxyphenyl)methylene]-1H-indene-1,3(2H)-dione
2-[3-(4-Chlorobenzoyl)-4-oxo-4H-furo[3,2-c][1 ]benzopyran-2-yl]phenylmethylene]-1H-
indene-1,3(2H)-dione
2-[(3-Benzoyl-4-oxo-4H-furo[3,2-c][1]benzopyran-2-yl)(4-bromophenyl)methylene]-1H-
indene-1,3(2H)-dione
1H-Indene-1,3(2H)-dione, 2-[6-(diphenylamino)-4,4-dimethyl-4H-indeno[1,2-b]furan-2-
yl]methylene]-
2-[[3-(4-Bromobenzoyl)-4-oxo-4H-furo[3,2-c][1 ]benzopyran-2-yl](4-
methoxyphenyl)methylene]-1H-indene-1,3(2H)-dione
2-[3-(4-Nitrobenzoyl)-4-oxo-4H-furo[3,2-c][1 ]benzopyran-2-yl]phenylmethylene]-1H-
indene-1,3(2H)-dione
2-[(3-Benzoyl-4-oxo-4H-furo[3,2-c][1]benzopyran-2-yl)(4-nitrophenyl)methylene]-1H-
indene-1,3(2H)-dione
2-[[3-(2-Methyl-1-oxopropyl)-4-oxo-4H-furo[3,2-c][1]benzopyran-2-yl](4-
nitrophenyl)methylene]-1H-indene-1,3(2H)-dione
2-[[3-(4-Methoxybenzoyl)-4-oxo-4H-furo[3,2-c][1]benzopyran-2-yl](4-
nitrophenyl)methylene]-1H-indene-1,3(2H)-dione
2-[[3-(4-Chlorobenzoyl)-4-oxo-4H-furo[3,2-c][1 ]benzopyran-2-yl](4-nitrophenyl)methylene]-
1H-indene-1,3(2H)-dione
1H-Indene-1,3(2H)-dione, 2-[[6-(diphenylamino)-4,4-dimethyl-4H-indeno[1,2-b]furan-2-
yl]methylene]-5,6-difluoro-
4-[5-[4,5-Dihydro-3-(hydroxymethoxymethylene)-2-methyl-5-oxo-3H-indeno[1,2-b ]pyridin-4-
yl]-2-furanyl]benzoic acid
3-Methyl 4-[5-(2-carboxyphenyl)-2-furanyl]-4,5-dihydro-2-methyl-5-oxo-1H-indeno[1,2-
b]pyridine-3-carboxylate
3-Methyl 4-[5-(4-carboxyphenyl)-2-furanyl]-4,5-dihydro-2-methyl-5-oxo-1H-indeno[1,2-
b]pyridine-3-carboxylate
3-[5-[2-Nitro-4-(trifluoromethyl)phenyl]-2-furanyl]indeno[1,2-c]pyrazol-4(1H)-one
1H-Indeno[1,2-b]pyridine-3-carboxylic acid, 4-[5-(3-carboxyphenyl)-2-furanyl]-4,5-dihydro-
2-methyl-5-oxo-, 3-methyl ester
1H-Indeno[1,2-b]pyridine-3-carboxylic acid, 4,5-dihydro-4-[5-[4-(methoxycarbonyl)phenyl]-
2-furanyl]-2-methyl-5-oxo-, methyl ester
1H-Indeno[1,2-b]pyridine-3-carboxylic acid, 4,5-dihydro-4-[5-(4-iodophenyl)-2-furanyl]-2-
methyl-5-oxo-, methyl ester
1H-Indeno[1,2-b]pyridine-3-carboxylic acid, 4-[5-(4-fluorophenyl)-2-furanyl]-4,5-dihydro-2-
methyl-5-oxo-, methyl ester
1H-Indeno[1,2-b]pyridine-3-carboxylic acid, 4-[5-(4-bromophenyl)-2-furanyl]-4,5-dihydro-2-
methyl-5-oxo-, methyl ester
Methyl 4-[5-(4-chlorophenyl)-2-furanyl]-4,5-dihydro-2-methyl-5-oxo-1H-indeno[1,2-
b]pyridine-3-carboxylate
Methyl 4-[5-(3-chlorophenyl)-2-furanyl]-4,5-dihydro-2-methyl-5-oxo-1H-indeno[1,2-
b]pyridine-3-carboxylate
1H-Indeno[1,2-b]pyridine-3-carboxylic acid, 4-[5-[4-(ethoxycarbonyl)phenyl]-2-furanyl]-4,5-
dihydro-2-methyl-5-oxo-, methyl ester
1H-Indeno[1,2-b]pyridine-3-carboxylic acid, 4,5-dihydro-2-methyl-4-[5-(4-nitrophenyl)-2-
furanyl]-5-oxo-, methyl ester
1H-Indeno[1,2-b]pyridine-3-carboxylic acid, 4,5-dihydro-2-methyl-4-[5-(3-nitrophenyl)-2-
furanyl]-5-oxo-, methyl ester
Methyl 4,5-dihydro-2-methyl-4-[5-(2-methyl-4-nitrophenyl)-2-furanyl]-5-oxo-1H-indeno[1,2-
blpyridine-3-carboxylate
1H-Indeno[1,2-b]pyridine-3-carboxylic acid, 4-[5-(2-bromo-4-methylphenyl)-2-furanyl]-4,5-
dihydro-2-methyl-5-oxo-, methyl ester
1H-Indeno[1,2-b]pyridine-3-carboxylic acid, 4,5-dihydro-2-methyl-4-[5-(2-methyl-5-
nitrophenyl)-2-furanyl]-5-oxo-, methyl ester
Methyl 4,5-dihydro-2-methyl-4-[5-(2-nitrophenyl)-2-furanyl]-5-oxo-1H-indeno[1,2-
b]pyridine-3-carboxylate
Methyl 4-[5-(3,4-dichlorophenyl)-2-furanyl]-4,5-dihydro-2-methyl-5-oxo-1H-indeno[1,2-
b]pyridine-3-carboxylate
1H-Indeno[1,2-b]pyridine-3-carboxylic acid, 4,5-dihydro-2-methyl-5-oxo-4-[5-[4-[(2-propyn-
1-ylamino)carbonyl]phenyl]-2-furanyl]-,methyl ester
1H-Indeno[1,2-b]pyridine-3-carboxylic acid, 4-[5-(2,3-dichlorophenyl)-2-furanyl]-4,5-
dihydro-2-methyl-5-oxo-, methyl ester
Methyl 4-[5-(2,4-dichlorophenyl)-2-furanyl]-4,5-dihydro-2-methyl-5-oxo-1H-indeno[1,2-
b]pyridine-3-carboxylate
3-Methyl 4-[5-(3-carboxy-4-chlorophenyl)-2-furanyl]-4,5-dihydro-2-methyl-5-oxo-1H-
indeno[1,2-b]pyridine-3-carboxylate
Methyl 4-[5-(2,5-dichlorophenyl)-2-furanyl]-4,5-dihydro-2-methyl-5-oxo-1H-indeno[1,2-
b]pyridine-3-carboxylate
Methyl 4-[5-(2-bromo-4-nitrophenyl)-2-furanyl]-4,5-dihydro-2-methyl-5-oxo-1H-indeno[1,2-
b]pyridine-3-carboxylate
1H-Indeno[1,2-b]pyridine-3-carboxylic acid, 4-[5-(2-chloro-4-nitrophenyl)-2-furanyl]-4,5-
dihydro-2-methyl-5-oxo-, methyl ester
1H-Indeno[1,2-b]pyridine-3-carboxylic acid, 4-[5-(4-chloro-3-nitrophenyl)-2-furanyl]-4,5-
dihydro-2-methyl-5-oxo-, methyl ester
Methyl 4,5-dihydro-4-[5-(2-methoxy-4-nitrophenyl)-2-furanyl]-2-methyl-5-oxo-1H-
indeno[1,2-b]pyridine-3-carboxylate
Methyl 4-[5-(2-chloro-5-nitrophenyl)-2-furanyl]-4,5-dihydro-2-methyl-5-oxo-1H-indeno[1,2-
blpyridine-3-carboxylate

### Further Forms of Compounds

In some aspects, a compound disclosed herein possesses one or more stereocenters and each stereocenter exists independently in either the R or S configuration. The compounds presented herein include all diastereomeric, enantiomeric, and epimeric forms as well as the appropriate mixtures thereof. The compounds and methods provided herein include all cis, trans, syn, anti, entgegen (E), and zusammen (Z) isomers as well as the appropriate mixtures thereof. In certain embodiments, compounds described herein are prepared as their individual stereoisomers by reacting a racemic mixture of the compound with an optically active resolving agent to form a pair of diastereoisomeric compounds/salts, separating the diastereomers and recovering the optically pure enantiomers. In some embodiments, resolution of enantiomers is carried out using covalent diastereomeric derivatives of the compounds described herein. In another embodiment, diastereomers are separated by separation/resolution techniques based upon differences in solubility. In other embodiments, separation of stereoisomers is performed by chromatography or by the forming diastereomeric salts and separation by recrystallization, or chromatography, or any combination thereof. Jean Jacques, Andre Collet, Samuel H. Wilen, “Enantiomers, Racemates and Resolutions”, John Wiley And Sons, Inc., 1981. In one aspect, stereoisomers are obtained by stereoselective synthesis.

In some embodiments, compounds described herein are prepared as prodrugs. A “prodrug” refers to an agent that is converted into the parent drug in vivo. Prodrugs are often useful because, in some situations, they may be easier to administer than the parent drug. They may, for instance, be bioavailable by oral administration whereas the parentis not. The prodrug may also have improved solubility in pharmaceutical compositions over the parent drug. In some embodiments, the design of a prodrug increases the effective water solubility. An example, without limitation, of a prodrug is a compound described herein, which is administered as an ester (the “prodrug”) to facilitate transmittal across a cell membrane where water solubility is detrimental to mobility but which then is metabolically hydrolyzed to the carboxylic acid, the active entity, once inside the cell where water-solubility is beneficial. A further example of a prodrug might be a short peptide (polyaminoacid) bonded to an acid group where the peptide is metabolized to reveal the active moiety. In certain embodiments, upon in vivo administration, a prodrug is chemically converted to the biologically, pharmaceutically or therapeutically active form of the compound. In certain embodiments, a prodrug is enzymatically metabolized by one or more steps or processes to the biologically, pharmaceutically or therapeutically active form of the compound.

In one aspect, prodrugs are designed to alter the metabolic stability or the transport characteristics of a drug, to mask side effects or toxicity, to improve the flavor of a drug or to alter other characteristics or properties of a drug.

In some embodiments, some of the herein-described compounds may be a prodrug for another derivative or active compound.

In some embodiments, sites on the aromatic ring portion of compounds described herein are susceptible to various metabolic reactions Therefore incorporation of appropriate substituents on the aromatic ring structures may reduce, minimize or eliminate this metabolic pathway. In specific embodiments, the appropriate substituent to decrease or eliminate the susceptibility of the aromatic ring to metabolic reactions is, by way of example only, a halogen, or an alkyl group.

In another embodiment, the compounds described herein are labeled isotopically (e.g., with a radioisotope) or by another other methods, including, but not limited to, the use of chromophores or fluorescent moieties, bioluminescent labels, or chemiluminescent labels.

Compounds described herein include isotopically-labeled compounds, which are identical to those recited in the various formulae and structures presented herein, but for the fact that one or more atoms are replaced by an atom having an atomic mass or mass number different from the atomic mass or mass number usually found in nature. Examples of isotopes that can be incorporated into the present compounds include isotopes of hydrogen, carbon, nitrogen, oxygen, sulfur, fluorine, chlorine, and iodine such as, for example, ²H, ³H, ¹³C, ¹⁴C, ¹⁵N, ¹⁸O, ¹⁷O, ³⁵S, ¹⁸F, ³⁶C₁, and ¹²⁵I. In one aspect, isotopically-labeled compounds described herein, for example those into which radioactive isotopes such as ³H and ¹⁴C are incorporated, are useful in drug and/or substrate tissue distribution assays. In one aspect, substitution with isotopes such as deuterium affords certain therapeutic advantages resulting from greater metabolic stability, such as, for example, increased in vivo half-life or reduced dosage requirements.

In additional or further embodiments, the compounds described herein are metabolized upon administration to an organism in need to produce a metabolite that is then used to produce a desired effect, including a desired therapeutic effect.

Compounds described herein may be formed as, and/or used as, pharmaceutically acceptable salts. The type of pharmaceutical acceptable salts, include, but are not limited to: (1) acid addition salts, formed by reacting the free base form of the compound with a pharmaceutically acceptable: inorganic acid, such as, for example, hydrochloric acid, hydrobromic acid, sulfuric acid, phosphoric acid, metaphosphoric acid, and the like; or with an organic acid, such as, for example, acetic acid, propionic acid, hexanoic acid, cyclopentanepropionic acid, glycolic acid, pyruvic acid, lactic acid, malonic acid, succinic acid, malic acid, maleic acid, fumaric acid, trifluoroacetic acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, toluenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, butyric acid, phenylacetic acid, phenylbutyric acid, valproic acid, and the like; (2) salts formed when an acidic proton present in the parent compound is replaced by a metal ion, e.g., an alkali metal ion (e.g., lithium, sodium, potassium), an alkaline earth ion (e.g., magnesium, or calcium), or an aluminum ion. In some cases, compounds described herein may coordinate with an organic base, such as, but not limited to, ethanolamine, diethanolamine, triethanolamine, tromethamine, N-methylglucamine, dicyclohexylamine, tris(hydroxymethyl)methylamine. In other cases, compounds described herein may form salts with amino acids such as, but not limited to, arginine, lysine, and the like. Acceptable inorganic bases used to form salts with compounds that include an acidic proton, include, but are not limited to, aluminum hydroxide, calcium hydroxide, potassium hydroxide, sodium carbonate, sodium hydroxide, and the like.

It should be understood that a reference to a pharmaceutically acceptable salt includes the solvent addition forms, particularly solvates. Solvates contain either stoichiometric or non-stoichiometric amounts of a solvent, and may be formed during the process of crystallization with pharmaceutically acceptable solvents such as water, ethanol, and the like. Hydrates are formed when the solvent is water, or alcoholates are formed when the solvent is alcohol. Solvates of compounds described herein can be conveniently prepared or formed during the processes described herein. In addition, the compounds provided herein can exist in unsolvated as well as solvated forms. In general, the solvated forms are considered equivalent to the unsolvated forms for the purposes of the compounds and methods provided herein.

### Methods of Synthesis

In some embodiments, the syntheses of compounds described herein are accomplished using means described in the chemical literature, using the methods described herein, or by a combination thereof. In addition, solvents, temperatures and other reaction conditions presented herein may vary.

In other embodiments, the starting materials and reagents used for the synthesis of the compounds described herein are synthesized or are obtained from commercial sources, such as, but not limited to, Sigma-Aldrich, Fisher Scientific (Fisher Chemicals), and Acros Organics.

In further embodiments, the compounds described herein, and other related compounds having different substituents are synthesized using techniques and materials described herein as well as those that are recognized in the field, such as described, for example, in Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, Advanced Organic Chemistry 4th Ed., (Wiley 1992); Carey and Sundberg, Advanced Organic Chemistry 4th Ed., Vols. A and B (Plenum 2000, 2001), and Green and Wuts, Protective Groups in Organic Synthesis 3rd Ed., (Wiley 1999) (all of which are incorporated by reference for such disclosure). General methods for the preparation of compounds as disclosed herein may be derived from reactions and the reactions may be modified by the use of appropriate reagents and conditions, for the introduction of the various moieties found in the formulae as provided herein. As a guide the following synthetic methods may be utilized.

In the reactions described, it may be necessary to protect reactive functional groups, for example hydroxy, amino, imino, thio or carboxy groups, where these are desired in the final product, in order to avoid their unwanted participation in reactions. A detailed description of techniques applicable to the creation of protecting groups and their removal are described in Greene and Wuts, Protective Groups in Organic Synthesis, 3rd Ed., John Wiley & Sons, New York, NY, 1999, and Kocienski, Protective Groups, Thieme Verlag, New York, NY, 1994, which are incorporated herein by reference for such disclosure).

It is understood that other analogous procedures and reagents could be used, and that these Schemes are only meant as non-limiting examples.

### Pharmaceutical Compositions

In one aspect, the compounds described herein are formulated into pharmaceutical compositions. Pharmaceutical compositions are formulated in a conventional manner using one or more pharmaceutically acceptable inactive ingredients that facilitate processing of the active compounds into preparations that can be used pharmaceutically. Proper formulation is dependent upon the route of administration chosen. A summary of pharmaceutical compositions described herein can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

A pharmaceutical composition, as used herein, refers to a mixture of a compound disclosed herein with other chemical components (i.e., pharmaceutically acceptable inactive ingredients), such as carriers, excipients, binders, filling agents, suspending agents, flavoring agents, sweetening agents, disintegrating agents, dispersing agents, surfactants, lubricants, colorants, diluents, solubilizers, moistening agents, plasticizers, stabilizers, penetration enhancers, wetting agents, anti-foaming agents, antioxidants, preservatives, or one or more combination thereof. The pharmaceutical composition facilitates administration of the compound to an organism.

Pharmaceutical formulations described herein are administrable to a subject in a variety of ways by multiple administration routes, including but not limited to, oral, parenteral (e.g., intravenous, subcutaneous, intramuscular, intramedullary injections, intrathecal, direct intraventricular, intraperitoneal, intralymphatic, intranasal injections), intranasal, buccal, topical or transdermal administration routes. The pharmaceutical formulations described herein include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations.

In some embodiments, the compounds disclosed herein are administered orally.

In some embodiments, the compounds disclosed herein are administered topically. In such embodiments, the compound disclosed herein is formulated into a variety of topically administrable compositions, such as solutions, suspensions, lotions, gels, pastes, shampoos, scrubs, rubs, smears, medicated sticks, medicated bandages, balms, creams or ointments. In one aspect, the compounds disclosed herein are administered topically to the skin.

In another aspect, the compounds disclosed herein are administered by inhalation.

In another aspect, the compounds disclosed herein are formulated for intranasal administration. Such formulations include nasal sprays, nasal mists, and the like.

In another aspect, the compounds disclosed herein are formulated as eye drops.

In any of the aforementioned aspects are further embodiments in which the effective amount of the compound disclosed herein is: (a) systemically administered to the mammal; and/or (b) administered orally to the mammal; and/or (c) intravenously administered to the mammal; and/or (d) administered by inhalation to the mammal; and/or (e) administered by nasal administration to the mammal; or and/or (f) administered by injection to the mammal; and/or (g) administered topically to the mammal; and/or (h) administered by ophthalmic administration; and/or (i) administered rectally to the mammal; and/or (j) administered non-systemically or locally to the mammal.

In any of the aforementioned aspects are further embodiments comprising single administrations of the effective amount of the compound disclosed herein, including further embodiments in which (i) the compound is administered once; (ii) the compound is administered to the mammal multiple times over the span of one day; (iii) the compound is administered continually; or (iv) the compound is administered continuously.

In any of the aforementioned aspects are further embodiments comprising multiple administrations of the effective amount of the compound disclosed herein, including further embodiments in which (i) the compound is administered continuously or intermittently: as in a single dose; (ii) the time between multiple administrations is every 6 hours; (iii) the compound is administered to the mammal every 8 hours; (iv) the compound is administered to the mammal every 12 hours; (v) the compound is administered to the mammal every 24 hours. In further or alternative embodiments, the method comprises a drug holiday, wherein the administration of the compound disclosed herein is temporarily suspended or the dose of the compound being administered is temporarily reduced; at the end of the drug holiday, dosing of the compound is resumed. In one embodiment, the length of the drug holiday varies from 2 days to 1 year.

In certain embodiments, the compound disclosed herein is administered in a local rather than systemic manner.

In some embodiments, the compound disclosed herein is administered topically. In some embodiments, the compound disclosed herein is administered systemically.

In some embodiments, the pharmaceutical formulation is in the form of a tablet. In other embodiments, pharmaceutical formulations of the compounds disclosed herein are in the form of a capsule.

In one aspect, liquid formulation dosage forms for oral administration are in the form of aqueous suspensions or solutions selected from the group including, but not limited to, aqueous oral dispersions, emulsions, solutions, elixirs, gels, and syrups.

For administration by inhalation, a compound disclosed herein is formulated for use as an aerosol, a mist or a powder.

For buccal or sublingual administration, the compositions may take the form of tablets, lozenges, or gels formulated in a conventional manner.

In some embodiments, compounds disclosed herein are prepared as transdermal dosage forms.

In one aspect, a compound disclosed herein is formulated into a pharmaceutical composition suitable for intramuscular, subcutaneous, or intravenous injection.

In some embodiments, the compound disclosed herein is be administered topically and can be formulated into a variety of topically administrable compositions, such as solutions, suspensions, lotions, gels, pastes, medicated sticks, balms, creams or ointments.

In some embodiments, the compounds disclosed herein are formulated in rectal compositions such as enemas, rectal gels, rectal foams, rectal aerosols, suppositories, jelly suppositories, or retention enemas.

### Methods of Dosing and Treatment Regimens

In one aspect, the compounds disclosed herein are used in the preparation of medicaments for the treatment of diseases or conditions described herein. In addition, a method for treating any of the diseases or conditions described herein in a subject in need of such treatment, involves administration of pharmaceutical compositions that include at least one compound disclosed herein or a pharmaceutically acceptable salt, active metabolite, prodrug, or solvate thereof, in therapeutically effective amounts to said subject.

In certain embodiments, the compositions containing the compound disclosed herein are administered for prophylactic and/or therapeutic treatments. In certain therapeutic applications, the compositions are administered to a patient already suffering from a disease or condition, in an amount sufficient to cure or at least partially arrest at least one of the symptoms of the disease or condition. Amounts effective for this use depend on the severity and course of the disease or condition, previous therapy, the patient's health status, weight, and response to the drugs, and the judgment of the treating physician. Therapeutically effective amounts are optionally determined by methods including, but not limited to, a dose escalation clinical trial.

In prophylactic applications, compositions containing the compounds disclosed herein are administered to a patient susceptible to or otherwise at risk of a particular disease, disorder or condition.

In certain embodiments, the dose of drug being administered may be temporarily reduced or temporarily suspended for a certain length of time (i.e., a “drug holiday”).

Doses employed for adult human treatment are typically in the range of 0.01 mg-5000 mg per day or from about 1 mg to about 1000 mg per day. In one embodiment, the desired dose is conveniently presented in a single dose or in divided doses.

In some embodiments, the dose is about 0.1 mg per day to about 5,000 mg per day. In some embodiments, the dose is about 0.1 mg per day to about 1 mg per day, about 0.1 mg per day to about 50 mg per day, about 0.1 mg per day to about 100 mg per day, about 0.1 mg per day to about 300 mg per day, about 0.1 mg per day to about 500 mg per day, about 0.1 mg per day to about 600 mg per day, about 0.1 mg per day to about 700 mg per day, about 0.1 mg per day to about 800 mg per day, about 0.1 mg per day to about 900 mg per day, about 0.1 mg per day to about 1,000 mg per day, about 0.1 mg per day to about 5,000 mg per day, about 1 mg per day to about 50 mg per day, about 1 mg per day to about 100 mg per day, about 1 mg per day to about 300 mg per day, about 1 mg per day to about 500 mg per day, about 1 mg per day to about 600 mg per day, about 1 mg per day to about 700 mg per day, about 1 mg per day to about 800 mg per day, about 1 mg per day to about 900 mg per day, about 1 mg per day to about 1,000 mg per day, about 1 mg per day to about 5,000 mg per day, about 50 mg per day to about 100 mg per day, about 50 mg per day to about 300 mg per day, about 50 mg per day to about 500 mg per day, about 50 mg per day to about 600 mg per day, about 50 mg per day to about 700 mg per day, about 50 mg per day to about 800 mg per day, about 50 mg per day to about 900 mg per day, about 50 mg per day to about 1,000 mg per day, about 50 mg per day to about 5,000 mg per day, about 100 mg per day to about 300 mg per day, about 100 mg per day to about 500 mg per day, about 100 mg per day to about 600 mg per day, about 100 mg per day to about 700 mg per day, about 100 mg per day to about 800 mg per day, about 100 mg per day to about 900 mg per day, about 100 mg per day to about 1,000 mg per day, about 100 mg per day to about 5,000 mg per day, about 300 mg per day to about 500 mg per day, about 300 mg per day to about 600 mg per day, about 300 mg per day to about 700 mg per day, about 300 mg per day to about 800 mg per day, about 300 mg per day to about 900 mg per day, about 300 mg per day to about 1,000 mg per day, about 300 mg per day to about 5,000 mg per day, about 500 mg per day to about 600 mg per day, about 500 mg per day to about 700 mg per day, about 500 mg per day to about 800 mg per day, about 500 mg per day to about 900 mg per day, about 500 mg per day to about 1,000 mg per day, about 500 mg per day to about 5,000 mg per day, about 600 mg per day to about 700 mg per day, about 600 mg per day to about 800 mg per day, about 600 mg per day to about 900 mg per day, about 600 mg per day to about 1,000 mg per day, about 600 mg per day to about 5,000 mg per day, about 700 mg per day to about 800 mg per day, about 700 mg per day to about 900 mg per day, about 700 mg per day to about 1,000 mg per day, about 700 mg per day to about 5,000 mg per day, about 800 mg per day to about 900 mg per day, about 800 mg per day to about 1,000 mg per day, about 800 mg per day to about 5,000 mg per day, about 900 mg per day to about 1,000 mg per day, about 900 mg per day to about 5,000 mg per day, or about 1,000 mg per day to about 5,000 mg per day. In some embodiments, the dose is about 0.1 mg per day, about 1 mg per day, about 50 mg per day, about 100 mg per day, about 300 mg per day, about 500 mg per day, about 600 mg per day, about 700 mg per day, about 800 mg per day, about 900 mg per day, about 1,000 mg per day, or about 5,000 mg per day. In some embodiments, the dose is at least about 0.1 mg per day, about 1 mg per day, about 50 mg per day, about 100 mg per day, about 300 mg per day, about 500 mg per day, about 600 mg per day, about 700 mg per day, about 800 mg per day, about 900 mg per day, or about 1,000 mg per day. In some embodiments, the dose is at most about 1 mg per day, about 50 mg per day, about 100 mg per day, about 300 mg per day, about 500 mg per day, about 600 mg per day, about 700 mg per day, about 800 mg per day, about 900 mg per day, about 1,000 mg per day, or about 5,000 mg per day.

In some embodiments, the dose is about 1 mg per day to about 1,000 mg per day. In some embodiments, the dose is about 1 mg per day to about 50 mg per day, about 1 mg per day to about 100 mg per day, about 1 mg per day to about 200 mg per day, about 1 mg per day to about 300 mg per day, about 1 mg per day to about 400 mg per day, about 1 mg per day to about 500 mg per day, about 1 mg per day to about 600 mg per day, about 1 mg per day to about 700 mg per day, about 1 mg per day to about 800 mg per day, about 1 mg per day to about 900 mg per day, about 1 mg per day to about 1,000 mg per day, about 50 mg per day to about 100 mg per day, about 50 mg per day to about 200 mg per day, about 50 mg per day to about 300 mg per day, about 50 mg per day to about 400 mg per day, about 50 mg per day to about 500 mg per day, about 50 mg per day to about 600 mg per day, about 50 mg per day to about 700 mg per day, about 50 mg per day to about 800 mg per day, about 50 mg per day to about 900 mg per day, about 50 mg per day to about 1,000 mg per day, about 100 mg per day to about 200 mg per day, about 100 mg per day to about 300 mg per day, about 100 mg per day to about 400 mg per day, about 100 mg per day to about 500 mg per day, about 100 mg per day to about 600 mg per day, about 100 mg per day to about 700 mg per day, about 100 mg per day to about 800 mg per day, about 100 mg per day to about 900 mg per day, about 100 mg per day to about 1,000 mg per day, about 200 mg per day to about 300 mg per day, about 200 mg per day to about 400 mg per day, about 200 mg per day to about 500 mg per day, about 200 mg per day to about 600 mg per day, about 200 mg per day to about 700 mg per day, about 200 mg per day to about 800 mg per day, about 200 mg per day to about 900 mg per day, about 200 mg per day to about 1,000 mg per day, about 300 mg per day to about 400 mg per day, about 300 mg per day to about 500 mg per day, about 300 mg per day to about 600 mg per day, about 300 mg per day to about 700 mg per day, about 300 mg per day to about 800 mg per day, about 300 mg per day to about 900 mg per day, about 300 mg per day to about 1,000 mg per day, about 400 mg per day to about 500 mg per day, about 400 mg per day to about 600 mg per day, about 400 mg per day to about 700 mg per day, about 400 mg per day to about 800 mg per day, about 400 mg per day to about 900 mg per day, about 400 mg per day to about 1,000 mg per day, about 500 mg per day to about 600 mg per day, about 500 mg per day to about 700 mg per day, about 500 mg per day to about 800 mg per day, about 500 mg per day to about 900 mg per day, about 500 mg per day to about 1,000 mg per day, about 600 mg per day to about 700 mg per day, about 600 mg per day to about 800 mg per day, about 600 mg per day to about 900 mg per day, about 600 mg per day to about 1,000 mg per day, about 700 mg per day to about 800 mg per day, about 700 mg per day to about 900 mg per day, about 700 mg per day to about 1,000 mg per day, about 800 mg per day to about 900 mg per day, about 800 mg per day to about 1,000 mg per day, or about 900 mg per day to about 1,000 mg per day. In some embodiments, the dose is about 1 mg per day, about 50 mg per day, about 100 mg per day, about 200 mg per day, about 300 mg per day, about 400 mg per day, about 500 mg per day, about 600 mg per day, about 700 mg per day, about 800 mg per day, about 900 mg per day, or about 1,000 mg per day. In some embodiments, the dose is at least about 1 mg per day, about 50 mg per day, about 100 mg per day, about 200 mg per day, about 300 mg per day, about 400 mg per day, about 500 mg per day, about 600 mg per day, about 700 mg per day, about 800 mg per day, or about 900 mg per day. In some embodiments, the dose is at most about 50 mg per day, about 100 mg per day, about 200 mg per day, about 300 mg per day, about 400 mg per day, about 500 mg per day, about 600 mg per day, about 700 mg per day, about 800 mg per day, about 900 mg per day, or about 1,000 mg per day.

In some embodiments, the dose is about 0.1 mg/kg to about 200 mg/kg. In some embodiments, the dose is about 0.1 mg/kg to about 1 mg/kg, about 0.1 mg/kg to about 3 mg/kg, about 0.1 mg/kg to about 5 mg/kg, about 0.1 mg/kg to about 10 mg/kg, about 0.1 mg/kg to about 50 mg/kg, about 0.1 mg/kg to about 70 mg/kg, about 0.1 mg/kg to about 90 mg/kg, about 0.1 mg/kg to about 120 mg/kg, about 0.1 mg/kg to about 150 mg/kg, about 0.1 mg/kg to about 200 mg/kg, about 1 mg/kg to about 3 mg/kg, about 1 mg/kg to about 5 mg/kg, about 1 mg/kg to about 10 mg/kg, about 1 mg/kg to about 50 mg/kg, about 1 mg/kg to about 70 mg/kg, about 1 mg/kg to about 90 mg/kg, about 1 mg/kg to about 120 mg/kg, about 1 mg/kg to about 150 mg/kg, about 1 mg/kg to about 200 mg/kg, about 3 mg/kg to about 5 mg/kg, about 3 mg/kg to about 10 mg/kg, about 3 mg/kg to about 50 mg/kg, about 3 mg/kg to about 70 mg/kg, about 3 mg/kg to about 90 mg/kg, about 3 mg/kg to about 120 mg/kg, about 3 mg/kg to about 150 mg/kg, about 3 mg/kg to about 200 mg/kg, about 5 mg/kg to about 10 mg/kg, about 5 mg/kg to about 50 mg/kg, about 5 mg/kg to about 70 mg/kg, about 5 mg/kg to about 90 mg/kg, about 5 mg/kg to about 120 mg/kg, about 5 mg/kg to about 150 mg/kg, about 5 mg/kg to about 200 mg/kg, about 10 mg/kg to about 50 mg/kg, about 10 mg/kg to about 70 mg/kg, about 10 mg/kg to about 90 mg/kg, about 10 mg/kg to about 120 mg/kg, about 10 mg/kg to about 150 mg/kg, about 10 mg/kg to about 200 mg/kg, about 50 mg/kg to about 70 mg/kg, about 50 mg/kg to about 90 mg/kg, about 50 mg/kg to about 120 mg/kg, about 50 mg/kg to about 150 mg/kg, about 50 mg/kg to about 200 mg/kg, about 70 mg/kg to about 90 mg/kg, about 70 mg/kg to about 120 mg/kg, about 70 mg/kg to about 150 mg/kg, about 70 mg/kg to about 200 mg/kg, about 90 mg/kg to about 120 mg/kg, about 90 mg/kg to about 150 mg/kg, about 90 mg/kg to about 200 mg/kg, about 120 mg/kg to about 150 mg/kg, about 120 mg/kg to about 200 mg/kg, or about 150 mg/kg to about 200 mg/kg. In some embodiments, the dose is about 0.1 mg/kg, about 1 mg/kg, about 3 mg/kg, about 5 mg/kg, about 10 mg/kg, about 50 mg/kg, about 70 mg/kg, about 90 mg/kg, about 120 mg/kg, about 150 mg/kg, or about 200 mg/kg. In some embodiments, the dose is at least about 0.1 mg/kg, about 1 mg/kg, about 3 mg/kg, about 5 mg/kg, about 10 mg/kg, about 50 mg/kg, about 70 mg/kg, about 90 mg/kg, about 120 mg/kg, or about 150 mg/kg. In some embodiments, the dose is at most about 1 mg/kg, about 3 mg/kg, about 5 mg/kg, about 10 mg/kg, about 50 mg/kg, about 70 mg/kg, about 90 mg/kg, about 120 mg/kg, about 150 mg/kg, or about 200 mg/kg.

### EXAMPLES

### Abbreviations

AML, acute myeloid leukemia; B-ALL, B cell acute lymphoblastic leukemia; BTLA, B- and T-lymphocyte attenuator; CDI, 1,1′-carbonyldiimidazole; CETSA, cellular thermal shift assay; CTLA-4, cytotoxic T-lymphocyte-associated protein 4; DIEA, N,N-diisopropylethylamine; DiFMUP, 6,8-difluoro-4-methylumbelliferyl phosphate; DME, dimethoxyethane; DMSO, dimethyl sulfoxide; DTT, dithiothreitol; EDC, 1-ethyl-3-(3-dimethylaminopropyl)carbodiimide; EDTA, ethylenediaminetetraacetic acid; ERK, extracellular signal-regulated kinase; GDP, guanosine diphosphate; gRNA, guide RNA; GST, glutathione S-transferase; GTP, guanosine triphosphate; HOBt, hydroxybenzotriazole; IRS-1, insulin receptor substrate 1; JAK, Janus kinase; JMIL, juvenile myelomonocytic leukemia; KO, knock-out; LDA, lithium diisopropylamide; Ni-NTA, nickel-nitrilotriacetic acid; OMFP, 3-O-methylfluoresceinphosphate; PBMC, Peripheral blood mononuclear cell; PD-1, Programmed cell death protein 1; PTK, protein tyrosine kinase; PTP, protein tyrosine pho sphatase; PTS, protein thermal shift; pTyr, phosphotyrosine; PyClU, chlorodipyrrolidinocarbenium hexafluorophosphate; RAF, rapidly acceleratedfibrosarcoma; RT, room temperature; RTK, receptor tyrosine kinase; SAR, structure activity relationship; SD, standard deviation; sgRNA, single guide RNA; SHP2, Src homology 2 domain-containing phosphatase 2; STAT, signal transducer and activator of transcription; STEP, striatal-enriched tyrosine phosphatase; TCEP, (tris(2-carboxyethyl)phosphine); THF, tetrahydrofuran; TKI, tyrosine kinase inhibitor; Tm, melting temperature.

### Novel Furanylbenzamides as SHP2 Inhibitors and Synthesis of Analogs

To identify novel SHP2 inhibitor scaffolds, an in-house small molecule library collection Table 2 was screened against one of the most frequent SHP2 oncogenic variants, E76K, using a protein thermal shift (PTS) assay. Confirmed PTS hits were then subjected to in vitro phosphatase inhibition assays using SHP2-E76K. Among the identified hits, one compound that contained a benzothiophenone-furanylbenzamide scaffold (Table 2, #02) showed good inhibitory activity against SHP2-E76K with an IC₅₀ value of 1.8 mM. This compound was selected for structure-activity relationship (SAR) and mechanistic studies. Interestingly, the benzothiophenone-furanylbenzamide scaffold of #02 lacked an obvious phosphotyrosine (pTyr)-mimicking group and did not contain any other charged moiety that may reduce cell membrane permeability. Importantly, compounds in this series did not act as covalent inhibitors via Michael addition to the enone double bond.

Using the general synthetic strategy shown in Scheme 1, a variety of benzothiophenone-furanylbenzamides were synthesized along with related analogs for subsequent testing in SHP2 phosphatase assays (Table 2). The first step to access key intermediate III involved reacting equimolar quantities of N,N-diethyl-2-(methylthio)benzamide I and 5-bromo-2-furaldehyde II. The resulting intermediate III was subjected to Suzuki cross-coupling reaction with the appropriate arylboronic acids IV to provide the target analogs V. The desired benzamides VI were prepared from the corresponding benzoic acid derivatives V using standard peptide coupling with appropriate amines. The tetrazole derivative #04 was accessible via azide cycloaddition from benzonitrile #05. A total of 48 compounds were synthesized and subsequently characterized (Table 2). All compounds had a purity of >95%.

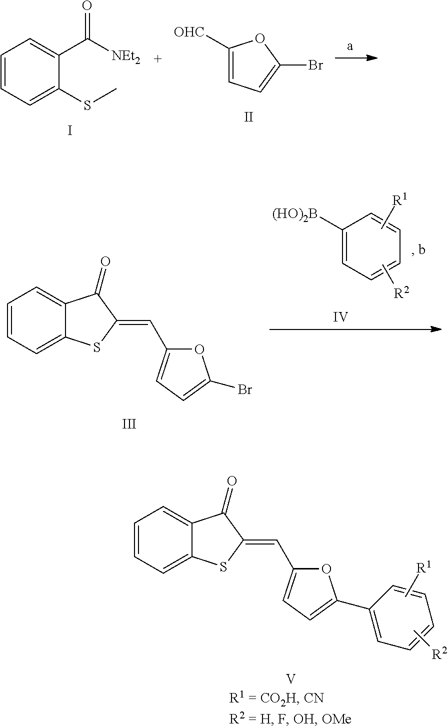

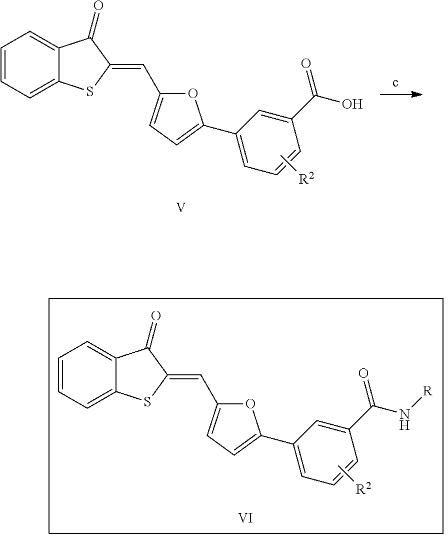

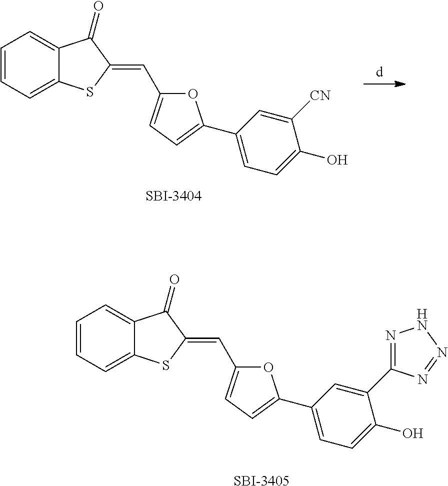 [SMILES: N#Cc1cc(-c2ccc(/C=C3\Sc4ccccc4C3=O)o2)ccc1O ; "SBI-3404" ; O=C1/C(=C/c2ccc(-c3ccc(O)c(-c4nn[nH]n4)c3)o2)Sc2ccccc21 ; "SBI-3405"]

Scheme 1. General synthetic scheme to access benzothiophenone-furanylbenzamides and analogs. α-lithiation of N,N-diethyl-2-(methylthio)benzamide I with LDA in dry THF followed by intramolecular alkylation and displacement of the diethylamide group afforded the benzothiophen-3-one in situ, which underwent an aldol condensation with the aldehyde II to deliver intermediate III. III was then subjected to Suzuki cross-coupling reaction with appropriate arylboronic acids IV to provide the target analogs V. The desired benzamides 6 were prepared by treating the benzoic acid derivatives V with appropriate amines in the presence of EDCI and HOBt in N,N-dimethylformamide. To afford the derivative in which the amide group is replaced with a tetrazole, benzonitrile #05 was heated with sodium azide in the presence of ammonium chloride in N,N-dimethylformamide to afford analog #04. (a) LDA, THF, 0° C.-rt, 2 h. (b) Pd(PPh₃)₄, 2 M Na₂CO₃, DME, 80° C., 6-12 h. (c) HOBt, EDC.HCl, R—NH₂, Et₃N, DMF, rt, 3 h. (d) NaN₃, NH₄Cl, DMF, 100° C., 1 h.

TABLE 2
SAR of example furanylbenzamides and analogs

| | | SHP2- | | |
|---|---|---|---|---|
| | | E76K | | |
| | | IC₅₀, | SHP2-WT | SHP2cat |
| Compound # | Structure | μM | IC₅₀, μM | IC₅₀, μM |
| #01 | 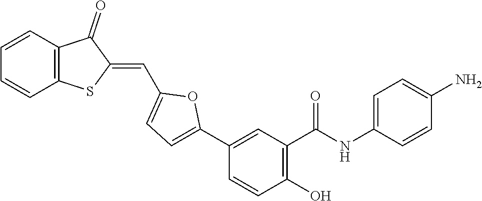 [SMILES: Nc1ccc(NC(=O)c2cc(-c3ccc(/C=C4\Sc5ccccc5C4=O)o3)ccc2O)cc1] | A | B | A |
| #02 | 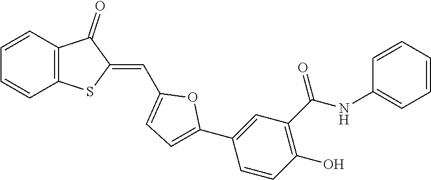 [SMILES: O=C(Nc1ccccc1)c1cc(-c2ccc(/C=C3\Sc4ccccc4C3=O)o2)ccc1O] | B | B | A |
| #03 | 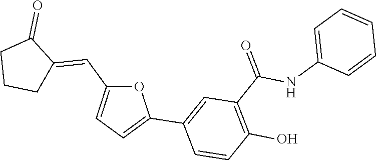 [SMILES: O=C1CCC/C1=C\c1ccc(-c2ccc(O)c(C(=O)Nc3ccccc3)c2)o1] | B | B | B |
| #04 | 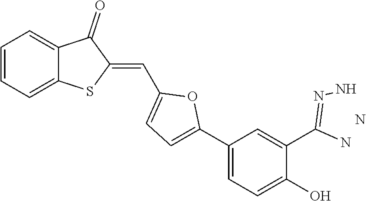 | B | B | B |
| #05 | 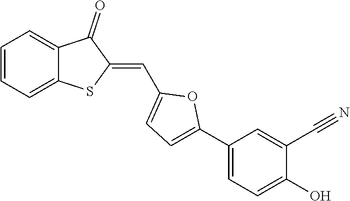 [SMILES: N#Cc1cc(-c2ccc(/C=C3\Sc4ccccc4C3=O)o2)ccc1O] | B | B | B |
| #06 | 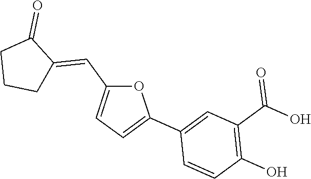 [SMILES: O=C1CCC/C1=C\c1ccc(-c2ccc(O)c(C(=O)O)c2)o1] | B | C | B |
| #07 | 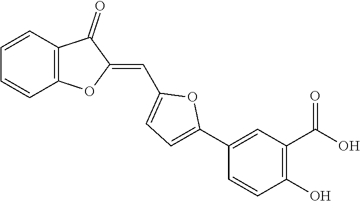 [SMILES: O=C(O)c1cc(-c2ccc(/C=C3\Oc4ccccc4C3=O)o2)ccc1O] | B | B | B |
| #08 | 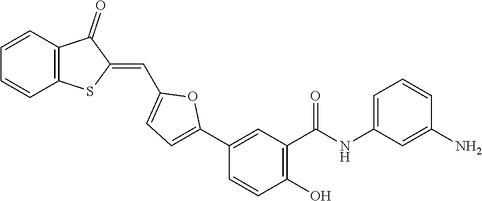 [SMILES: Nc1cccc(NC(=O)c2cc(-c3ccc(/C=C4\Sc5ccccc5C4=O)o3)ccc2O)c1] | B | B | B |
| #09 | 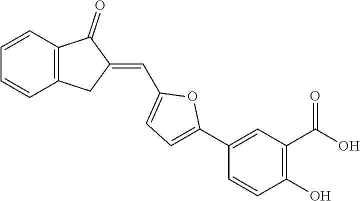 [SMILES: O=C(O)c1cc(-c2ccc(/C=C3\Cc4ccccc4C3=O)o2)ccc1O] | B | B | B |
| #10 | 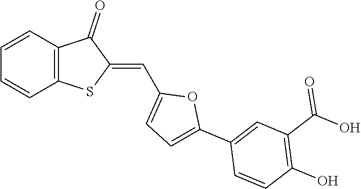 [SMILES: O=C(O)c1cc(-c2ccc(/C=C3\Sc4ccccc4C3=O)o2)ccc1O] | B | B | B |
| #11 | 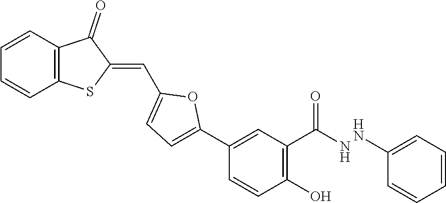 [SMILES: O=C(NNc1ccccc1)c1cc(-c2ccc(/C=C3\Sc4ccccc4C3=O)o2)ccc1O] | B | B | B |
| #12 | 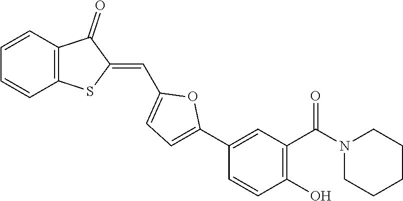 [SMILES: O=C1/C(=C/c2ccc(-c3ccc(O)c(C(=O)N4CCCCC4)c3)o2)Sc2ccccc21] | B | C | B |
| #13 | 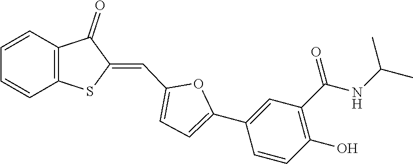 [SMILES: CC(C)NC(=O)c1cc(-c2ccc(/C=C3\Sc4ccccc4C3=O)o2)ccc1O] | B | C | n/d |
| #14 | 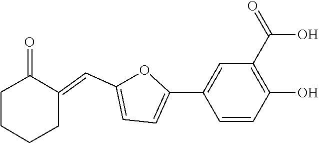 [SMILES: O=C1CCCC/C1=C\c1ccc(-c2ccc(O)c(C(=O)O)c2)o1] | C | C | n/d |
| #15 | 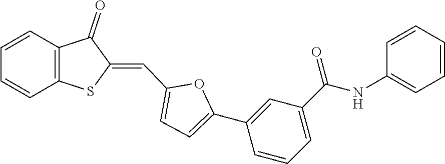 [SMILES: O=C(Nc1ccccc1)c1cccc(-c2ccc(/C=C3\Sc4ccccc4C3=O)o2)c1] | C | C | n/d |
| #16 | 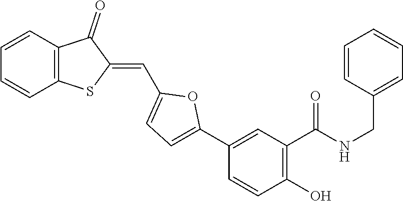 [SMILES: O=C(NCc1ccccc1)c1cc(-c2ccc(/C=C3\Sc4ccccc4C3=O)o2)ccc1O] | C | D | n/d |
| #17 | 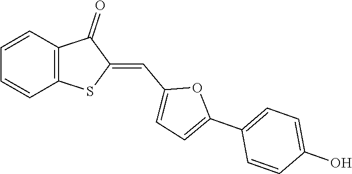 [SMILES: O=C1/C(=C/c2ccc(-c3ccc(O)cc3)o2)Sc2ccccc21] | C | D | n/d |
| #18 | 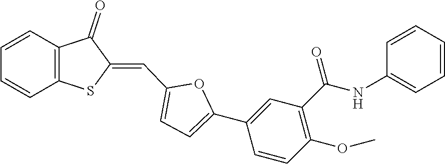 [SMILES: COc1ccc(-c2ccc(/C=C3\Sc4ccccc4C3=O)o2)cc1C(=O)Nc1ccccc1] | C | C | n/d |
| #19 | 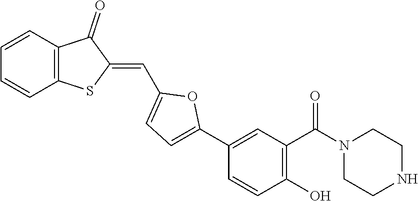 [SMILES: O=C1/C(=C/c2ccc(-c3ccc(O)c(C(=O)N4CCNCC4)c3)o2)Sc2ccccc21] | C | D | n/d |
| #20 | 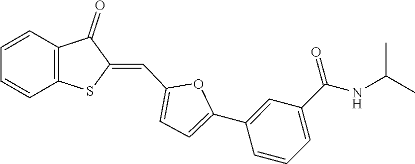 [SMILES: CC(C)NC(=O)c1cccc(-c2ccc(/C=C3\Sc4ccccc4C3=O)o2)c1] | D | D | n/d |
| #21 | 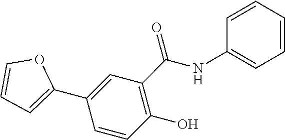 [SMILES: O=C(Nc1ccccc1)c1cc(-c2ccco2)ccc1O] | D | D | n/d |
| #22 | 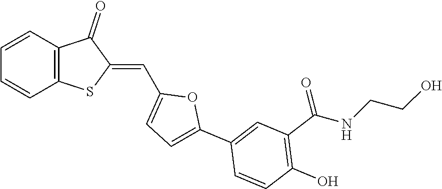 [SMILES: O=C(NCCO)c1cc(-c2ccc(/C=C3\Sc4ccccc4C3=O)o2)ccc1O] | D | D | n/d |
| #23 | 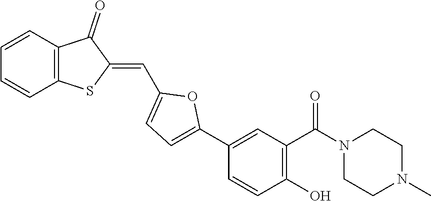 [SMILES: CN1CCN(C(=O)c2cc(-c3ccc(/C=C4\Sc5ccccc5C4=O)o3)ccc2O)CC1] | D | D | n/d |
| #24 | 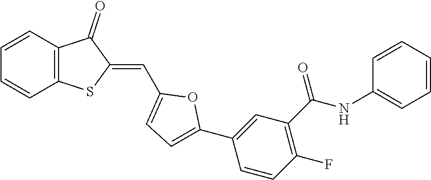 [SMILES: O=C(Nc1ccccc1)c1cc(-c2ccc(/C=C3\Sc4ccccc4C3=O)o2)ccc1F] | D | D | n/d |
| #25 | 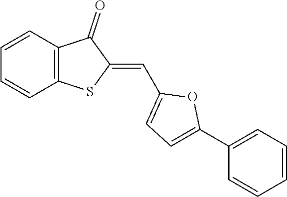 [SMILES: O=C1/C(=C/c2ccc(-c3ccccc3)o2)Sc2ccccc21] | D | D | n/d |
| #26 | 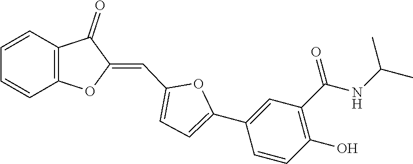 [SMILES: CC(C)NC(=O)c1cc(-c2ccc(/C=C3\Oc4ccccc4C3=O)o2)ccc1O] | B | B | n/d |
| #27 | 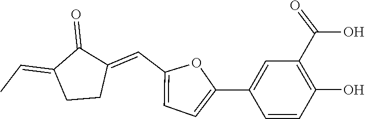 [SMILES: C/C=C1\CC/C(=C\c2ccc(-c3ccc(O)c(C(=O)O)c3)o2)C1=O] | B | B | n/d |
| #28 | 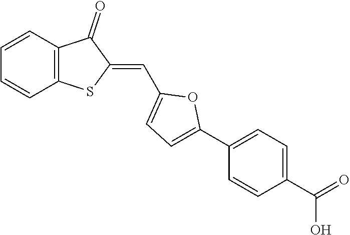 [SMILES: O=C(O)c1ccc(-c2ccc(/C=C3\Sc4ccccc4C3=O)o2)cc1] | B | B | n/d |
| #29 | 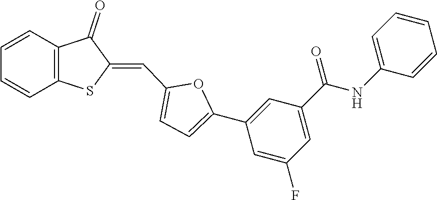 [SMILES: O=C(Nc1ccccc1)c1cc(F)cc(-c2ccc(/C=C3\Sc4ccccc4C3=O)o2)c1] | B | C | n/d |
| #30 | 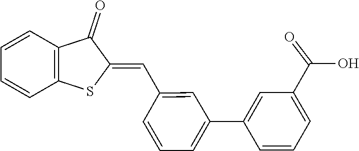 [SMILES: O=C(O)c1cccc(-c2cccc(/C=C3\Sc4ccccc4C3=O)c2)c1] | B | B | n/d |
| #31 | 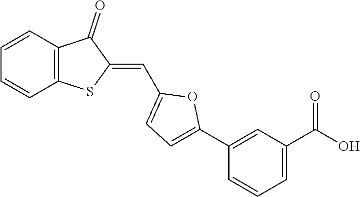 [SMILES: O=C(O)c1cccc(-c2ccc(/C=C3\Sc4ccccc4C3=O)o2)c1] | B | B | n/d |
| #32 | 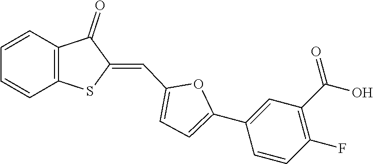 [SMILES: O=C(O)c1cc(-c2ccc(/C=C3\Sc4ccccc4C3=O)o2)ccc1F] | B | C | n/d |
| #33 | 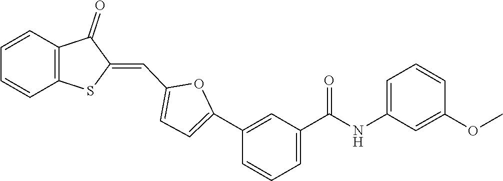 [SMILES: COc1cccc(NC(=O)c2cccc(-c3ccc(/C=C4\Sc5ccccc5C4=O)o3)c2)c1] | B | C | n/d |
| #34 | 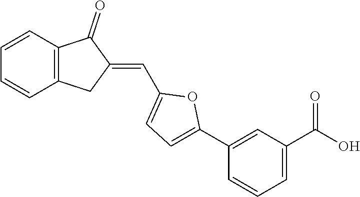 [SMILES: O=C(O)c1cccc(-c2ccc(/C=C3\Cc4ccccc4C3=O)o2)c1] | B | C | n/d |
| #35 | 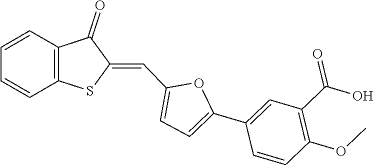 [SMILES: COc1ccc(-c2ccc(/C=C3\Sc4ccccc4C3=O)o2)cc1C(=O)O] | B | C | n/d |
| #36 | 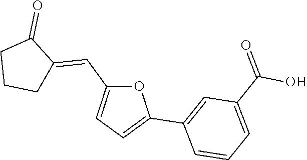 [SMILES: O=C1CCC/C1=C\c1ccc(-c2cccc(C(=O)O)c2)o1] | C | C | n/d |
| #37 | 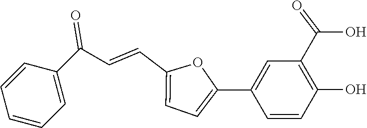 [SMILES: O=C(/C=C/c1ccc(-c2ccc(O)c(C(=O)O)c2)o1)c1ccccc1] | C | C | n/d |
| #38 | 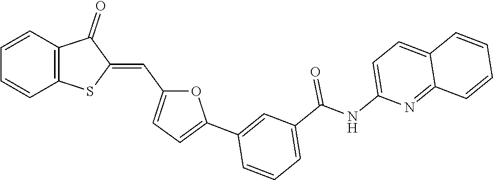 [SMILES: O=C(Nc1ccc2ccccc2n1)c1cccc(-c2ccc(/C=C3\Sc4ccccc4C3=O)o2)c1] | C | D | n/d |
| #39 | 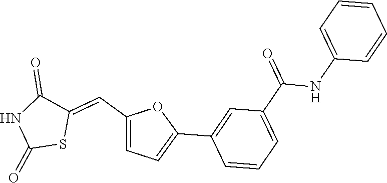 [SMILES: O=C1NC(=O)/C(=C/c2ccc(-c3cccc(C(=O)Nc4ccccc4)c3)o2)S1] | C | C | n/d |
| #40 | 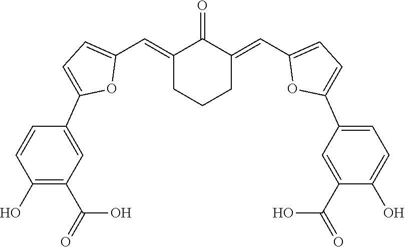 [SMILES: O=C1/C(=C/c2ccc(-c3ccc(O)c(C(=O)O)c3)o2)CCC/C1=C\c1ccc(-c2ccc(O)c(C(=O)O)c2)o1] | A | A | A |
| #41 | 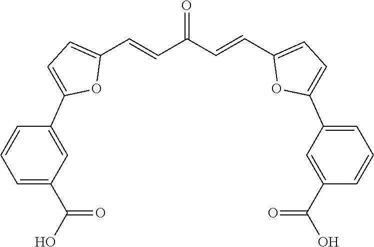 [SMILES: O=C(/C=C/c1ccc(-c2cccc(C(=O)O)c2)o1)/C=C/c1ccc(-c2cccc(C(=O)O)c2)o1] | A | A | A |
| #42 | 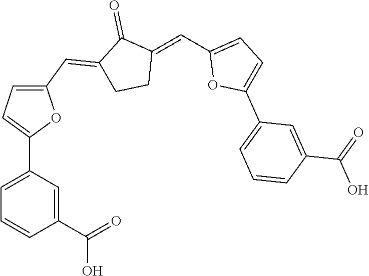 [SMILES: O=C1/C(=C/c2ccc(-c3cccc(C(=O)O)c3)o2)CC/C1=C\c1ccc(-c2cccc(C(=O)O)c2)o1] | A | A | A |
| #43 | 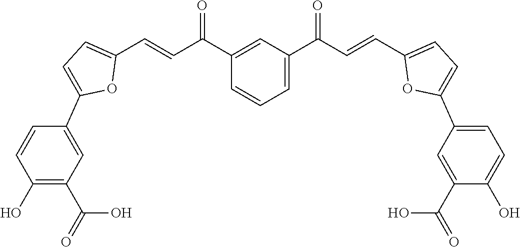 [SMILES: O=C(/C=C/c1ccc(-c2ccc(O)c(C(=O)O)c2)o1)c1cccc(C(=O)/C=C/c2ccc(-c3ccc(O)c(C(=O)O)c3)o2)c1] | A | B | A |
| #44 | 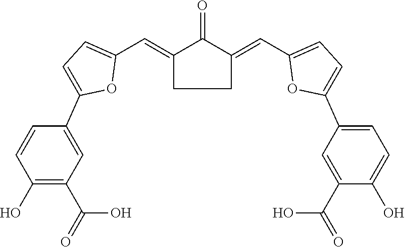 [SMILES: O=C1/C(=C/c2ccc(-c3ccc(O)c(C(=O)O)c3)o2)CC/C1=C\c1ccc(-c2ccc(O)c(C(=O)O)c2)o1] | A | B | n/d |
| #45 | 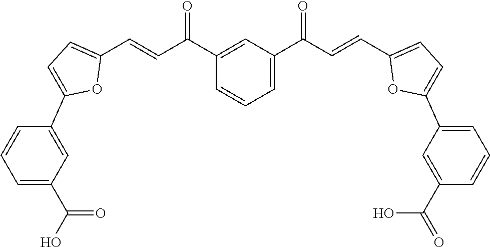 [SMILES: O=C(O)c1cccc(-c2ccc(/C=C/C(=O)c3cccc(C(=O)/C=C/c4ccc(-c5cccc(C(=O)O)c5)o4)c3)o2)c1] | B | B | B |
| #46 | 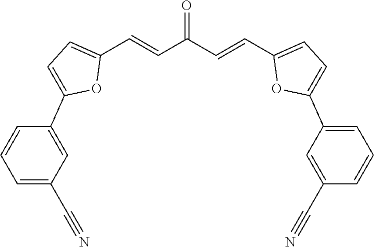 [SMILES: N#Cc1cccc(-c2ccc(/C=C/C(=O)/C=C/c3ccc(-c4cccc(C#N)c4)o3)o2)c1] | B | B | B |
| #47 | 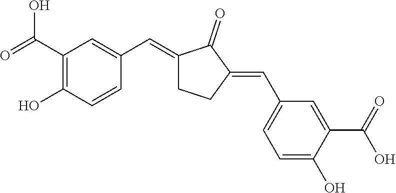 [SMILES: O=C1/C(=C/c2ccc(O)c(C(=O)O)c2)CC/C1=C\c1ccc(O)c(C(=O)O)c1] | B | B | n/d |
| #48 | 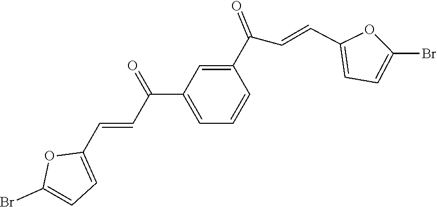 [SMILES: O=C(/C=C/c1ccc(Br)o1)c1cccc(C(=O)/C=C/c2ccc(Br)o2)c1] | C | C | n/d |

A: IC₅₀ is 1 ≤ μM;
B: IC₅₀ is >1 μM and ≤20 μM;
C: IC₅₀ > 20 μM and <100 μM;
D: IC₅₀ ≥ 100 μM;
and n/d is not tested.

### SAR and Biochemical Evaluation of Furanylbenzamides

To test the synthesized compounds for their potential to inhibit SHP2 activity, a fluorescence intensity-based assay using 6,8-difluoro-4-methylumbelliferyl phosphate (DiFMUP) as the substrate was adapted for three recombinant SHP2 constructs: 1) the SHP2 catalytic domain (SHP2cat; residues 248-527), 2) the full-length SHP2-E76K oncogenic mutant, and 3) the full-length SHP2 wild-type (SHP2-WT). The recombinant SHP2 proteins were expressed and purified. A dually phosphorylated peptide derived from the insulin receptor substrate 1 (IRS-1) served as a surrogate binding protein and was used to activate SHP2-WT. The constitutively active E76K mutant did not require activation. Similarly, the SHP2cat construct, which lacks the SH2 domains, did not need to be activated. Michaelis-Menten experiments to determine the DiFMUP Michaelis-Menten constant (Kₘn) for each SHP2 construct were performed and yielded the following values: SHP2-WT, Kₘ=60 μM; SHP2-E76K, Kₘ=20 μM; SHP2cat, Kₘ=20 μM. Relative maximum rates (Vₘₐₓ) of the DiFMUP reactions were as follows: SHP2-WT, Vₘₐₓ=871 AFU/min; SHP2-E76K, Vₘₐₓ, =2730 AFU/min; SHP2cat, Vₘₐₓ=2912 AFU/min. IC₅₀ values for each compound were determined from initial rates in 10-point dose-response assays using DiFMUP at a concentration corresponding to its Kₘ value for the respective SHP2 construct. The most active compounds exhibited sub- or low micromolar activity against SHP2 (Table 2). Active compounds generally inhibited all three SHP2 constructs, suggesting that they act on the SHP2 catalytic domain.

### Mechanism-of-Action (MOA) and Mode of Inhibition Studies

Furanylsalicylic acids are potent and selective inhibitors of the Yersinia tyrosine phosphatase Yop. It has been shown that the negatively charged salicylate moiety can act as a pTyr-mimetic and undergoes strong hydrogen bonding interactions with the phosphate-binding loop (P-loop) at the catalytic center, including a salt bridge with the guanidinium group of an invariant arginine that is part of the PTP signature motif (C(X)₅R) and conserved among all PTPs. Thus, we tested whether replacement of the neutral 2-hydroxybenzamide moiety with the negatively charged salicylate would be beneficial for the potency of this scaffold against SHP2. Interestingly, as mentioned above, the salicylate analogs, including #06, #07, #09, #10, and #14, were less potent than the N-phenyl-2-hydroxybenzamides. These results suggested that the benzamide moiety likely does not act as a pTyr-mimetic or interact with the P-loop residues in SHP2.

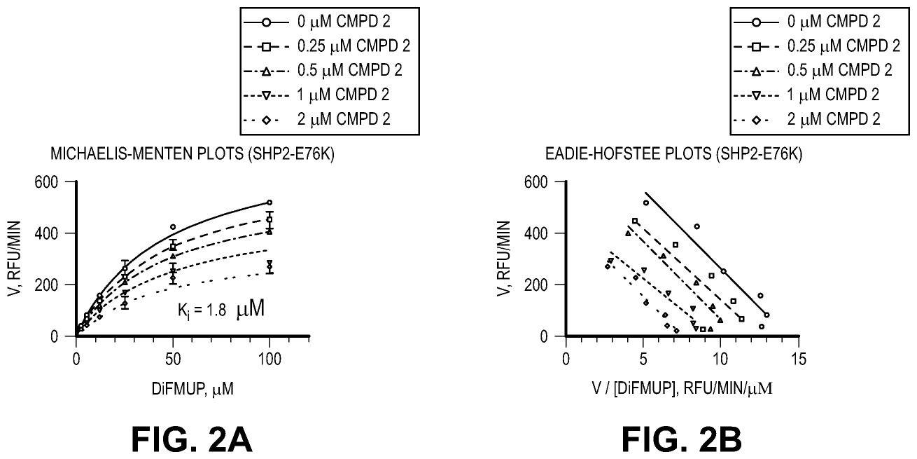

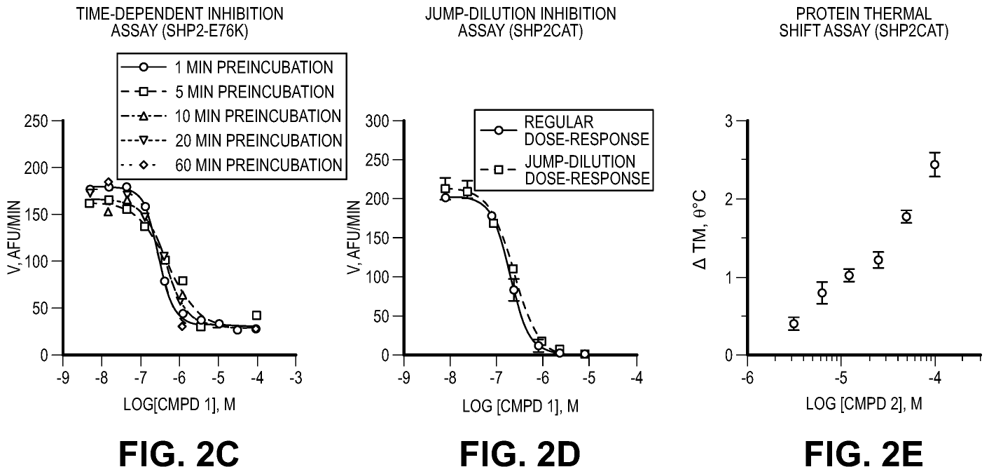

To corroborate this postulate, we subjected #02 to Michaelis-Menten kinetic studies with SHP2-E76K to determine its mode of inhibition (FIG. 2A,B). FIG. 2 shows A) Michaelis-Menten kinetic studies for the SHP2 inhibitor #02 with SHP2-E76K. Plots show the initial rates (V) at various substrate (DiFMUP) and inhibitor concentrations fitted to the Michaelis-Menten equation for noncompetitive inhibition. B) Eadie-Hofstee plots of the Michaelis-Menten kinetic studies with compound #02. D) Dose-response curves for #01 with SHP2-E76K after various preincubation times of inhibitor with SHP2. No time-dependent inhibition was observed as demonstrated by the similar potency for the various time points. D) Dose-response curves for #01 with SHP2cat with or without a 10× inhibitor/protein preincubation and jump-dilution. Identical IC₅₀ curves indicate that #01 is a reversible inhibitor. E) Dose-dependent binding of #02 to SHP2cat in a protein thermal shift (PTS) assay. Thermal stabilization of SHP2 by #02 is shown by the increase in the SHP2 melting temperature (ΔTₘ) compared to vehicle control (DMSO).

Using nonlinear regression, initial rates at various inhibitor and substrate concentrations were fitted to the Michaelis-Menten equations for competitive, noncompetitive, uncompetitive, and mixed inhibition. Fitting models were then compared using Akaike's Information Criterion (AIC), and inhibition mode probabilities were calculated from the differences between the corresponding second-order corrected AIC scores. The probability data unambiguously indicated that #02 does not directly compete with substrate binding. Similarly, an uncompetitive inhibition mode was found to be unlikely. Instead, the AIC probability data clearly suggested that #02 inhibits SHP2 either by a noncompetitive or mixed inhibition mechanism. The inhibition constant (Kᵢ) for noncompetitive inhibition was calculated to be 1.8 μM for #02, which corresponded well with the measured IC₅₀ value.

PTPs contain a highly nucleophilic cysteine that is essential for catalytic activity, but is susceptible to oxidation and covalent modifications which abrogate its nucleophilic function and phosphatase activity. Because the methylenebenzothiophenone moiety of our inhibitors could potentially act as a Michael acceptor and thus covalently bind to the catalytic cysteine, we tested whether the observed inhibition of SHP2 was dependent on the preincubation time of inhibitor and enzyme. An irreversible covalent inhibitor may show time-dependent inhibition, with increased potency at longer incubation times. Conversely, the potency of a non-covalent inhibitor is expected to be insensitive to the time it is incubated with the enzyme. To test for time-dependent inhibition, #01 (#01) at 10 different concentrations was preincubated with SHP2-E76K for 1, 5, 10, 20, or 60 min, before the DiFMUP substrate was added to the reaction mixture and initial rates were recorded (FIG. 2C). The dose-response curves overlapped very well and IC₅₀ values were very similar, suggesting that inhibition of SHP2 by #01 is not time-dependent. Similar results were obtained for #02.

Additionally, we used mass spectrometry to test whether inhibitor: protein adducts are formed. Specifically, we incubated SHP2cat (5 μM) with #02 (100 μM) in assay buffer for various times (1, 5, 20, and 60 min) before trypsin proteolysis and subsequent mass spectrometry analysis. While the coverage was good, and peptides comprising all existing cysteine residues, including the catalytic cysteine, were detected, no adduct formation with the inhibitor was found.

Finally, we performed a jump-dilution experiment to test for irreversible inhibition of SHP2. #01 was preincubated for 10 min with SHP2cat at 10× inhibitor and protein concentrations, compared to a regular dose-response experiment, followed by a 10×jump-dilution, incubation for an additional 10 min, and addition of the DiFMUP substrate. An irreversible inhibitor is expected to shift the IC₅₀ curve to lower IC₅₀ values in the jump-dilution experiment due to the higher inhibitor concentration during preincubation. In our experiment, in which we tested #01 in parallel in a jump-dilution and a regular dose-response assay, IC₅₀ curves were practically identical (FIG. 2D).

Collectively, these data suggest that our inhibitors do not act by irreversibly modifying the catalytic cysteine or any other amino acid of SHP2.

### Biophysical Binding and Inhibitor Confirmation in Orthogonal Assays

We confirmed specific and dose-dependent binding of inhibitors to SHP2 by protein thermal shift, which monitors the thermal stability of a target protein in vitro. Using recombinant SHP2cat in a PTS assay, #02 dose-dependently increased the melting temperature (Tₘ) of SHP2cat compared to the vehicle (DMSO) control (FIG. 2D). The data confirm biophysical binding of #02 to the SHP2 protein within the catalytic domain and agree with the results from the enzyme inhibition assays. In addition, we confirmed the inhibitory activity of several furanylbenzamide inhibitors, including the top two compounds #02 and #01, in SHP2cat inhibition assays using the alternative substrate 3-O-methylfluorescein phosphate (OMFP), which features a peak fluorescence emission that is red-shifted by −75 nm compared to DiFMUP. The Kₘ value of OMFP for SHP2cat was determined to be 50 μM. An OMFP fluorescence intensity-based assay was used to perform inhibitor dose-response experiments and determine IC₅₀ values. Inhibitor potencies were found to be comparable with those determined using DiFMUP. IC₅₀ values of #02 or #01 against SHP2cat with OMFP were determined to be 0.44 μM and 1.2 μM, respectively. Collectively, these data demonstrate genuine binding and inhibition of SHP2 by our inhibitors.

### Selectivity of Furanylbenzamides for SHP2

Selectivity is one of the biggest challenges in PTP inhibitor development. Therefore, we evaluated potent furanylbenzamides for their ability to selectively inhibit SHP2 over the closely related phosphatases PTP1B and striatal-enriched tyrosine phosphatase (STEP) (Table 3). Similar to SHP2, we adapted a 384-well plate format DiFMUP assay for recombinant PTP1B and STEP and performed Michaelis-Menten experiments to determine DiFMUP Kₘ values for each phosphatase. IC₅₀ values of our inhibitors for PTP1B and STEP were determined in 10-point dose-response assays with DiFMUP used at the concentration corresponding to the respective Kₘ value. Judging from the IC₅₀ values against the catalytic domain of each phosphatase, the most potent SHP2 inhibitor, #01, also exhibited the best selectivity for SHP2 (31-fold selective over PTP1B; 200-fold selective over STEP). Other inhibitors with good relative selectivity for SHP2 included #02 (13-fold selective over PTP1B; 73-fold selective over STEP) and #05 (5-fold selective over PTP1B; >71-fold selective over STEP). Taken together, these results suggest the furanylbenzamide inhibitors are not pan-active phosphatase inhibitors but instead exhibit a promising level of selectivity for SHP2.

TABLE 3
Selectivity of furanylbenzamide inhibitors for SHP2 over
related phosphatases PTP1B and STEP (IC₅₀ values in uM;
each PTP construct was comprised of the catalytic domain)

| | #01 | #02 | #03 | #05 | #04 |
|---|---|---|---|---|---|
| SHP2 | 0.22 | 0.73 | 1.1 | 1.4 | 2.0 |
| PTP1B | 6.8 | 9.4 | 1.9 | 7.4 | 5.3 |
| STEP | 44 | 53 | 27 | >100 | 7.7 |

### Evaluation of Furanylbenzamides in Wild-Type SHP2 Cellular Cancer Models and in Patient-Derived AML Samples

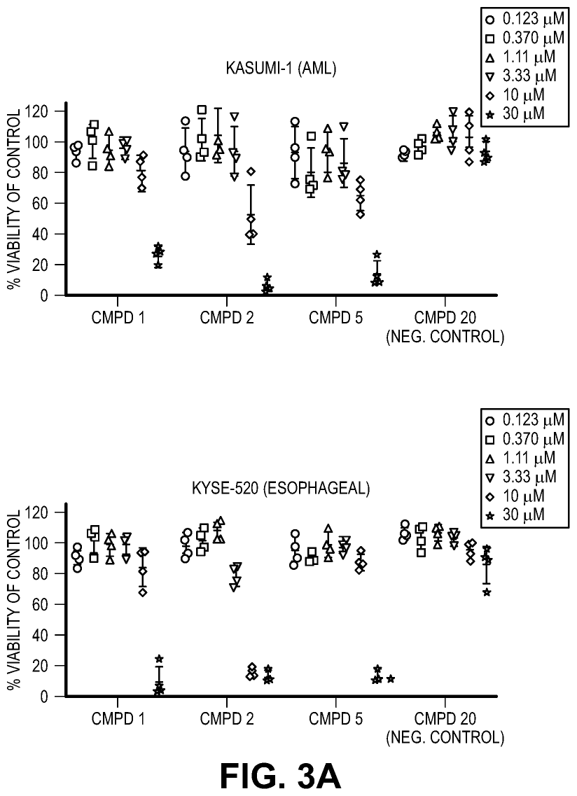

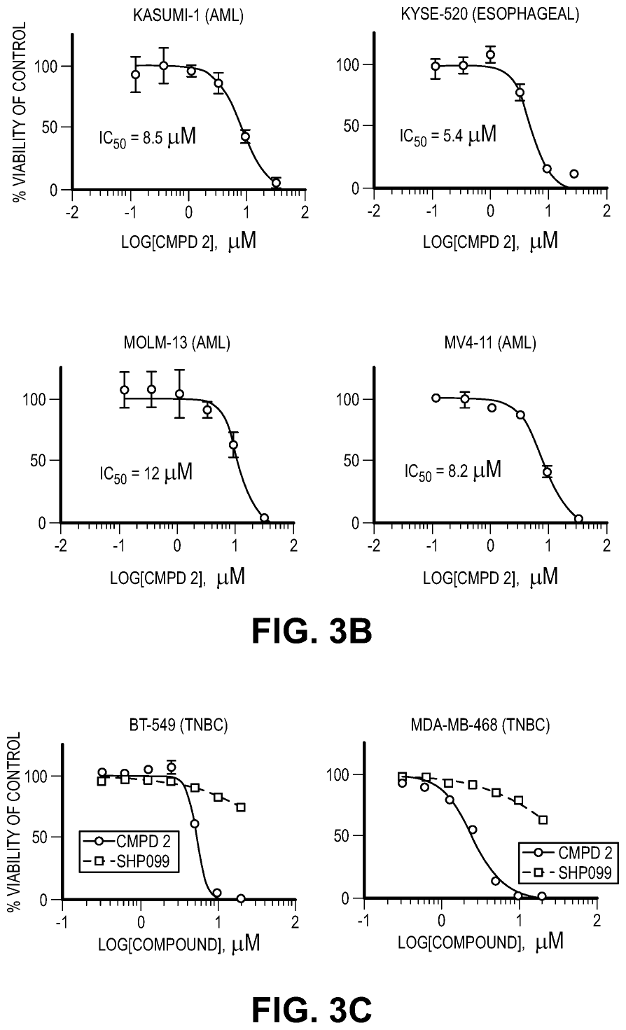

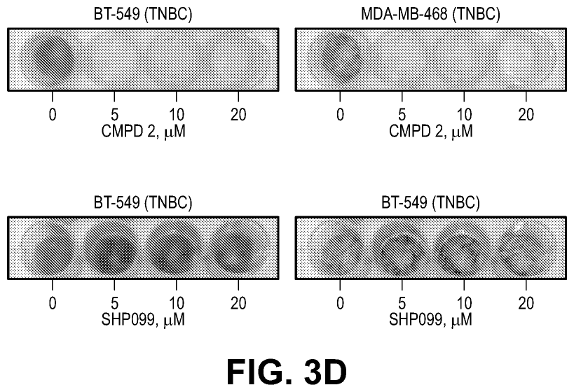

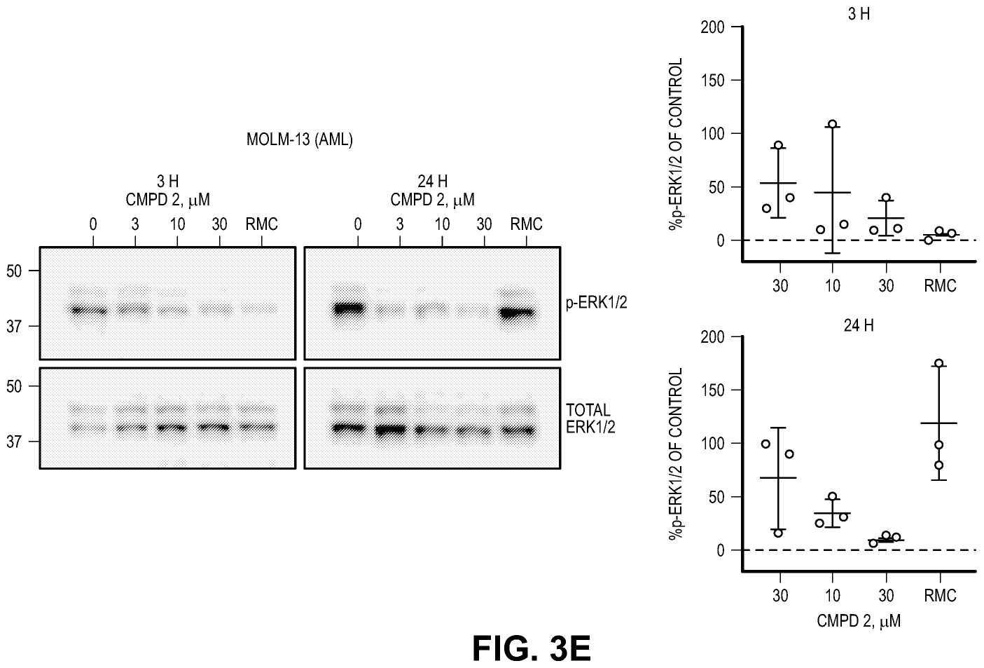

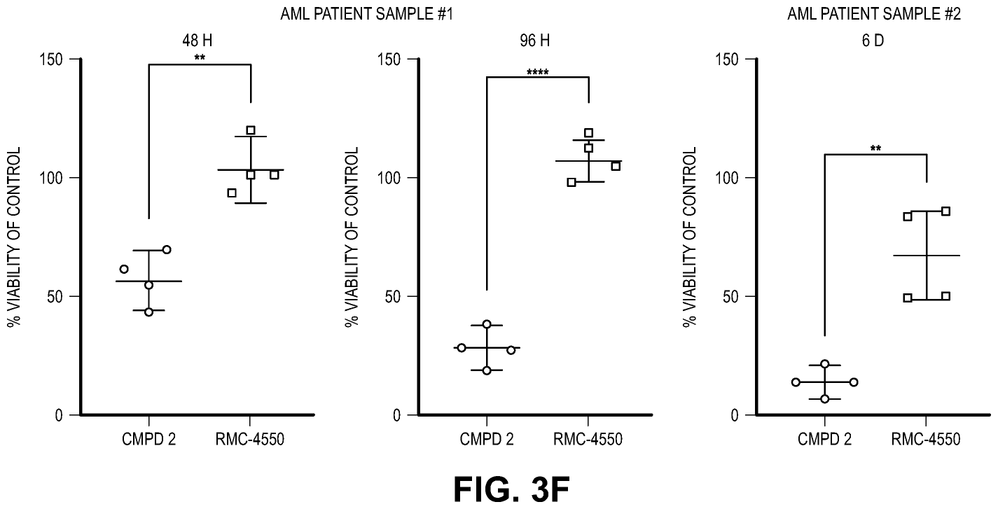

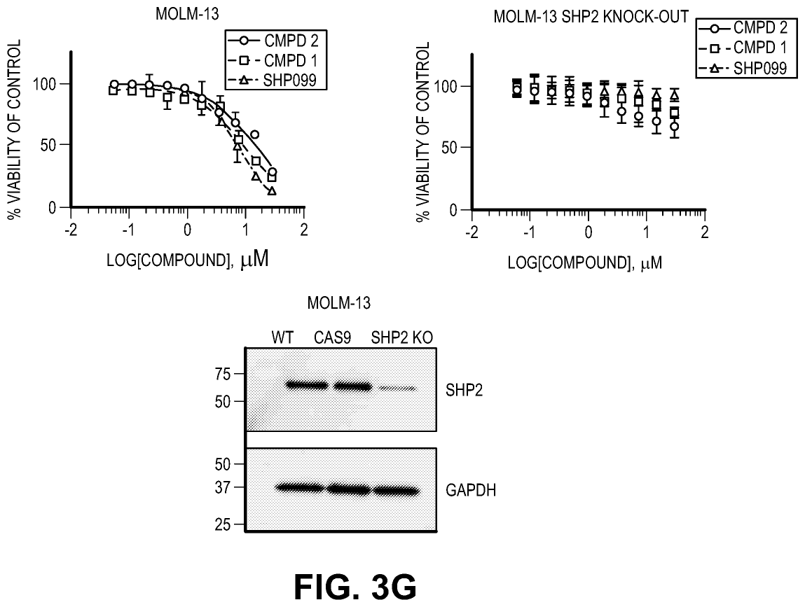

Inhibition of SHP2 has been reported to inhibit the growth of cancer cells including Kasumi-1 acute myeloid leukemia (AML) and KYSE-520 esophageal cancer cells. Thus, we tested our most potent and selective SHP2 inhibitors (#02, #01, and #05) in cell viability assays using the Kasumi-1 and KYSE-520 cell lines (FIG. 3A). We also included an inactive analog (#20; SHP2 IC₅₀>100 μM) as a negative control. We found that all three SHP2 inhibitors dose-dependently inhibited cancer cell growth, while the inactive analog showed no notable effects. #02 exhibited the greatest effect on cell growth in both Kasumi-1 cells (IC₅₀=8.5 μM) and KYSE-520 cells (IC₅₀=5.4 μM) (FIG. 3B). FIG. 3 shows A) Viability of acute myeloid leukemia (AML, Kasumi-1) and esophageal cancer (KYSE-520) cells in the presence of various concentrations of SHP2 active (#01, #02, and #05) and non-active (negative control) furanylbenzamides after 3 d in culture. Cell viability is shown as a percentage of the vehicle (DMSO) control and represents the mean±SD. B) #02 dose-response curves in cell viability assays using Kasumi-1, KYSE-520, MOLM-13, and MV4-1 cells after 3 d in culture. The percentages compared to DMSO vehicle control were curve fitted using nonlinear regression (log[inhibitor] vs. normalized response, variable slope) and represent the mean±SD. C) Cell viability of BT-459 and MDA-MB-468 triple-negative breast cancer (TNBC) cells in the presence of various concentrations of #02 or the SHP2 allosteric inhibitor SHP099 after 5 d in culture. Cell viability is shown as a percentage of DMSO vehicle control, representing mean±SD, and curve fitted as in B). D) Colony formation assay (11 d) of BT-459 and MDA-MB-468 TNBC cells in the presence of various concentrations of #02 or the SHP2 allosteric inhibitor SHP099. E) Phospho-ERK1/2 (p-ERK1/2) immunoblot analysis from total cell lysates of MOLM-13 AML cells treated with #02 at the indicated concentrations, or with SHP2 allosteric inhibitor RMC-4550 (RMC, 1 μM) for 3 h or 24 h. The quantitation of p-ERK1/2 levels is shown as the percentage of the DMSO (vehicle) control and represents data from three independent experiments (mean±SD). F) Viability of AML patient-derived cells in the presence of 10 μM #02 or 10 μM SHP2 allosteric inhibitor RMC-4550 after 2 d, 4 d, or 6 d in culture. Cell viability is shown as a percentage of the DMSO vehicle control and represents the mean±SD (*p<0.05; **p<0.01; ****p<0.0001; unpaired test with Welch's correction) G) Viability of MOLM-13 cells and MOLM-13-Cas9-mCherry cells with SHP2 knockout (KO) in the presence of #01, #02, and allosteric inhibitor SHP099 at various concentrations (10-point dose-response). The percentages compared to the DMSO vehicle control were curve fitted using nonlinear regression (log[inhibitor] vs. normalized response, variable slope) and represent the mean±SD. SHP2 protein levels in regular MOLM-13 cells (WT), MOLM-13-Cas9-mCherry cells (Cas9), and MOLM-13-Cas9-mCherry cells with SHP2 KO (SHP2 KO) were evaluated by immunoblot analysis using SHP2 antibodies.

A dose-dependent effect of #02 on cell viability was also determined in additional AML cell lines, including MOLM-13 (IC₅₀=12 μM) and MV4-11 (IC₅₀=8.2 μM). Several studies have revealed that SHP2 is upregulated or hyperactivated in breast cancer, including triple-negative breast cancer (TNBC) (16,42-44). Thus, we treated two TNBC cell lines, BT-549 and MDA-MB-468, with #02 or the SHP2 allosteric inhibitor SHP099 at various concentrations and determined cell viability after 72 h, compared to the vehicle control (FIG. 3C). #02 dose-dependently inhibited the growth of TNBC cells with IC₅₀ values of 5.4 μM (BT-549) and 2.5 μM (MDA-MB-468), respectively, while SHP099 had a very minor effect on TNBC cell viability. Next, we performed an 11-day colony formation assay with TNBC cells treated with #02 or SHP099 at 5, 10, or 20 μM (FIG. 3D). While vehicle-treated cells retained their capacity to produce colonies, colonies were undetectable for seeded cells treated with #02. As with the cell viability assays, the SHP2 allosteric inhibitor SHP099 exhibited a very weak effect on colony formation in the TNBC cell lines.

Because SHP2 activity is critical for ERK activation, we used phospho-ERK1/2 (p-ERK1/2) as a more direct readout of SHP2 inhibitor efficacy. MOLM-13 AML cells were treated with either #02 at 3, 10, or 30 μM or with RMC-4550, a highly potent SHP099-like SHP2 allosteric inhibitor, for 3 or 24 h, and p-ERK1/2 levels were detected by immunoblot analysis (FIG. 3E). RMC-4550 was used at 1 μM, a concentration that previously was shown to be effective in similar experiments. We found that #02 dose-dependently inhibits ERK1/2 activation in cells treated for either 3 or 24 h. However, a greater effect of #02 was observed after 24 h treatment. Interestingly, while RMC-4550 was highly effective in impeding ERK1/2 activation at the 3 h time point, p-ERK levels in MOLM-13 cells after 24 h treatment were similar to control cells treated with vehicle. This p-ERK rebound in MOLM-13 cells treated with RMC-4550 is reminiscent of previously reported data from human hepatoma Hep3B cells treated with SHP099.

We tested the effects of #02 orRMC-4550 on two patient-derived AML samples (FIG. 3F). #02 (10 μM) inhibited the growth of both AML patient samples, while RMC-4550 (10 μM) only affected the growth of sample 2, albeit to a lesser extent than #02. Collectively, these data demonstrate the efficacy of the novel inhibitors in various cell culture models of cancer, including in primary, patient-derived cells.

Finally, we evaluated the selectivity of our top two compounds, #01 and #02, on MOLM-13 AML cells in which SHP2 was depleted using CRISPR-Cas9 knock-out (KO). High-efficiency Cas9-editing MOLM-13 cells were generated by transducingMOLM-13 cells with a Cas9 lentiviral construct. Stable clones were tested for editing efficiency by performing TIDE analysis (45), and these MOLM13-Cas9 cells were transduced with a lentiviral construct containing an AAVS1 single guide RNA (sgRNA) and an mCherry reporter for bulk-sorting of cells with successful AAVS1 editing. SHP2 KO was achieved by transducing MOLM-13-Cas9-mCherry cells with a lentivirus containing SHP2 dual sgRNAs, resulting in ˜80% reduction of SHP2 protein levels (FIG. 3G). #01, #02, and the SHP2 allosteric inhibitor SHP099 were tested in parallel on regular MOLM-13 cells (expressing WT SHP2) and MOLM-13-Cas9-mCherry cells with SHP2 KO. Cells were treated with inhibitors at various concentrations (ranging from 30 mM to 58 nM) or vehicle control (DMSO) for 72 h, before cell viability was assessed. All three SHP2 inhibitors inhibited the cell viability of MOLM-13 cells with IC50 values of 15 mM (#02), 7.6 mM (#01), and 9.4 mM (SHP099). In contrast, the inhibitors had either no effect, or a greatly reduced effect, on the viability of MOLM-13-Cas9-mCherry SHP2 KO cells, demonstrating the selectivity of the compounds for SHP2 under physiological conditions (FIG. 3G). In summary, the data demonstrate both efficacy and selectivity of our inhibitors in cancer cells expressing WT SHP2.

### Evaluation of #02 in Oncogenic Mutant SHP2 Cellular Models

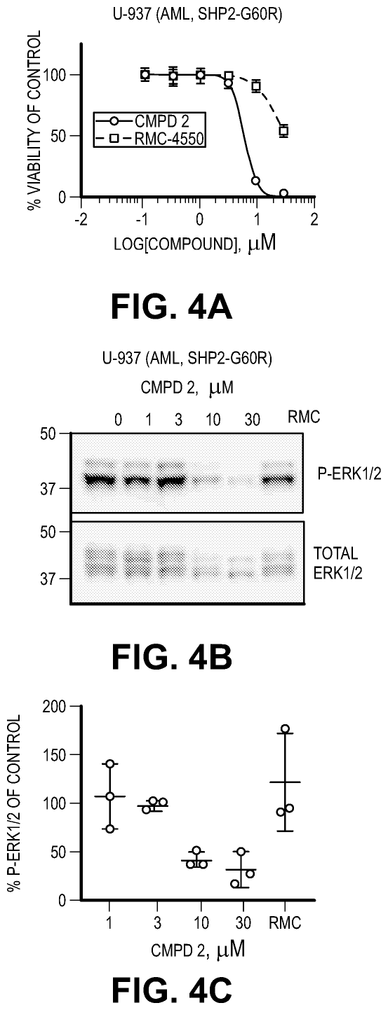

Oncogenic gain-of-function mutations in SHP2 drive leukemogenesis in a significant number of leukemia patients. However, due to their unique mechanism of action, the existing SHP2 allosteric inhibitors lack activity against the most common SHP2 oncogenic variants. Thus, we tested whether #02 can inhibit the growth of leukemia cells expressing oncogenic SHP2 variants. Importantly, we previously established the direct target engagement of #02 with mutant SHP2 in live cells using a cellular protein thermal shift assay (CETSA). Using the U-937 AML cell line, which harbors a G60R oncogenic mutation in SHP2, we treated cells with either #02 or RMC-4550 at various concentrations, ranging from 0.123 to 30 μM (FIG. 4A). In agreement with prior reports, the SHP099-like allosteric inhibitor RMC-4550 had a very weak effect on the viability of cells expressing the SHP2 gain-of-function variant (IC₅₀=33 μM). By contrast, #02 inhibited U-937 cell growth with an IC₅₀ value of 6.3 μM, which is comparable to the potency found in AML cells expressing WT SHP2. We also assessed the effects of #02 and RMC-4550 on ERK1/2 activation in the SHP2 mutant U-937 cell line. Similar to the immunoblot experiments described above, U-937 AML cells were treated with either vehicle control (DMSO), #02 (1, 3, 10, or 30 μM), or with RMC-4550 (1 μM) for 24 h and processed for p-ERK1/2 immunoblot analysis (FIG. 4B).

FIG. 4 shows A) Cell viability of U-937 AML cells in the presence of various concentrations of #02 or the SHP2 allosteric inhibitor RMC-4550 after 3 d in culture. Cell viability is shown as a percentage of the DMSO vehicle control, representing the mean±SD, and curve fitted using nonlinear regression (log[inhibitor] vs. normalized response, variable slope). B) Phospho-ERK1/2 (p-ERK1/2) immunoblot analysis from total cell lysates of U-937 AML cells treated with #02 at the indicated concentrations, or with SHP2 allosteric inhibitor RMC-4550 (RMC, 1 μM) for 24 h. The quantitation of p-ERK1/2 levels is shown as the percentage of the DMSO (vehicle) control and represents data from three independent experiments (mean SD).

While #02 dose-dependently inhibited ERK1/2 activation, a decrease in p-ERK1/2 levels was not detectable after treatment with RMC-4550. Collectively, our data demonstrate similar efficacy of #02 on AML cells expressing a common SHP2 oncogenic variant compared to AML cells expressing WT SHP2.

### Chemistry and Synthetic Procedures

All reactions were performed in oven-dried glassware under an atmosphere of nitrogen with magnetic stirring. All solvents and chemicals used were purchased from Sigma-Aldrich or Acros and were used as received. Purity and characterization of compounds were established by a combination of liquid chromatography-mass spectroscopy (LC-MS) and NMR analytical techniques and was >95% for all tested compounds. Silica gel column chromatography was carried out using prepacked silica cartridges from RediSep® (ISCO Ltd.) and eluted using an Isco Companion system. ¹H- and ¹³C-NMR spectra were obtained on a JEOL 400 spectrometer at 400 MHz and 100 MHz, respectively. Chemical shifts are reported in δ (ppm) relative to residual solvent peaks or TMS as internal standards. J-coupling constants are reported in Hz. High-resolution ESI-TOF mass spectra were acquired from the Mass Spectrometry Core at Sanford Burnham Prebys Medical Discovery Institute. HPLC-MS analyses were performed on a Shimadzu 2010EV LCMS using the following conditions: Kromisil® C18 column (reverse phase, 4.6 mm×50 mm); a linear gradient from 10% acetonitrile and 90% water to 95% acetonitrile and 5% water over 4.5 min; flow rate of 1 mL/min; UV photodiode array detection from 200 to 300 nm.

### (Z)-2-((5-Bromofuran-2-yl)methylene)benzo[b]thiophen-3(2H)-one (Intermediate III)

To a vigorously stirred solution of LDA (22 mmol) in anhydrous THE (25 mL) at 0° C., a solution of N,N-diethyl-2-(methylthio)benzamide (2.23 g, 10 mmol) and 5-bromo-2-furaldehyde (1.74 g, 10 mmol) in anhydrous THF (5 mL) was added under nitrogen atmosphere. The resulting mixture was stirred for 2 h, and then gradually warmed to room temperature. The reaction mixture was then poured into H₂O. The pH was adjusted to 4-5 by addition of diluted HCl. The organic layer was separated, and the aqueous layer was extracted with ethyl acetate. The organic phases were combined and dried over anhydrous Na₂SO₄. Evaporation of the solvent followed by silica gel chromatography using hexanes/ethyl acetate (20:1) yielded (Z)-2-((5-bromofuran-2-yl)methylene)benzo[b]thiophen-3(2H)-one III. Orange solid (2 g, 65.7%). ¹H NMR (400 MHz, CDCl₃): δ7.88 (d, J=7.8 Hz, 1H), 7.61 (s, 1H), 7.55 (t, J=7.8 Hz, 1H), 7.48 (d, J=7.8 Hz, 1H), 7.28 (t, J=7.3 Hz, 1H), 6.78 (d, J=3.7 Hz, 1H), 6.50 (d, J=3.7 Hz, 1H). ¹³CNMR (101 MHz, CDCl₃): δ188.2, 152.9, 146.1, 135.2, 130.6, 128.9, 126.9, 125.5, 123.9, 119.5, 117.9, 115.3. LC-MS (ESI) [M+H]⁺: 307.

### 2-Hydroxy-5-(5-((2-oxocyclopentylidene)methyl)furan-2-yl)-N-phenylbenzamide (#03)

To a stirred solution of 2-hydroxy-5-(5-((2-oxocyclopentylidene)methyl)furan-2-yl)benzoic acid (0.050 g, 0.17 mmol) in DMF (3 mL) was added aniline (0.023 g, 025 mmol), N,N-diisopropylethylamine (0.088 mL, 0.50 mmol), and chlorodipyrrolidinocarbenium hexafluorophosphate (PyClU) (0.084 g, 0.25 mmol) at room temperature. The resulting mixture was stirred at for 2 h. The reaction mixture was diluted with EtOAc and quenched with the addition of saturated NH₄Cl. The layers were separated, and the aqueous layer was washed with ethyl acetate three times. The combined organic layer was dried over anhydrous Na₂SO₄, concentrated to give the crude product which was purified by reverse phase HPLC. Dark red solid (0.033 g, 53%). ¹HNMR (400 MHz, DMSO-d₆): δ8.29 (t, J=2.7 Hz, 1H), 7.83 (dd, J=8.9, 2.5 Hz, 1H), 7.75-7.69 (m, 2H), 7.66 (d, J=8.0 Hz, 1H), 7.43-7.35 (m, 2H), 7.14 (t, J=6.9 Hz, 1H), 7.09-7.00 (m, 4H), 3.04 (dt, J=8.1, 4.2 Hz, 2H), 2.03-1.97 (m, 2H), 1.96-1.87 (m, 2H). LC-MS (ESI) [M+H]⁺:374.

### (Z)-2-((5-(4-Hydroxy-3-(2H-tetrazol-5-yl)phenyl)furan-2-yl)methylene)benzo[b]thiophen-3(2H)-one (#04)

(Z)-2-Hydroxy-5-(5-((3-oxobenzo[b]thiophen-2(3H)-ylidene)methyl)furan-2-yl)benzonitrile (0.070 g, 0.2 mmol), sodium azide (0.156 g, 2.4 mmol), and ammonium chloride (0.127 g, 2.4 mmol) were taken in DMF (4 mL) and the resultant mixture was heated at 100° C. for 1 h. The reaction mixture was cooled to room temperature and diluted with water and extracted with ethyl acetate (3×10 mL). The organic phases were combined and dried over anhydrous Na₂SO₄. Evaporation of the solvent followed by reverse phase HPLC yielded title compound. Dark red solid (0.049 g, 64%). ¹HNMR (400 MHz, DMSO-d₆): δ8.52 (d, J=2.3 Hz, 1H), 7.99-7.95 (m, 1H), 7.85 (d, J=7.8 Hz, 1H), 7.80 (d, J=7.8 Hz, 1H), 7.79 (s, 1H), 7.72 (t, J=8.2 Hz, 1H), 7.39 (t, J=7.8 Hz, 1H), 7.35 (d, J=3.7 Hz, 1H), 7.27 (d, J=3.7 Hz, 1H), 7.24 (d, J=7.8 Hz, 1H). LC-MS (ESI) [M+H]⁺: 389.

### (Z)—N-(3-aminophenyl)-2-hydroxy-5-(5-((3-oxobenzo[b]thiophen-2(3H)-ylidene)methyl)furan-2-yl)benzamide (#08)

(Z)-2-hydroxy-5-(5-((3-oxobenzo[b]thiophen-2(3H)-ylidene)methyl)furan-2-yl)benzoic acid (0.05 g, 0.137 mmol) and CDI (0.022 g, 0.137 mmol) were taken in DMF (2 mL) and stirred at for 1 h. to this benzene-1,3-diamine (0.030 g, 0.274 mmol) was added and stirring was continued for another 1-2 hr. After complete consumption of the starting material, the reaction mixture was diluted with water and extracted with EtOAc. The combined organic extracts were washed with water, dried over anhydrous Na₂SO₄ and concentrated under reduced pressure to yield the crude product which was further purified by reverse phase HPLC. Red solid (0.036 g, 58%). ¹H-NMR (400 MHz, DMSO-d₆) δ 10.23 (s, 1H), 8.23 (1H), 7.61-8.03 (9H), 7.24-7.40 (3H), 7.14 (s, 1H), 6.95 (d, J=8.2 Hz, 1H). ¹³C-NMR (101 MHz, DMSO-d₆) δ 187.4, 163.3, 160.1, 157.7, 157.2, 149.9, 145.9, 145.8, 136.0, 131.6, 131.5, 129.7, 127.0, 126.8, 126.6, 126.4, 126.2, 125.1, 124.9, 123.6, 123.4, 119.9, 109.5. LC-MS (ESI) [M+H]⁺: 455.

### (Z)-2-((5-(4-Hydroxyphenyl)furan-2-yl)methylene)benzo[b]thiophen-3(2H)-one (#17)

To a mixture of 2-((5-bromofuran-2-yl)methylene)benzo[b]thiophen-3(2H)-one (0.307 g, 1 mmol), (4-hydroxyphenylboronic acid (0.206 g, 1.5 mmol) and tetrakistriphenylphosphinepalladium(0) (0.115 g, 0.1 mmol) in DME (5 mL) was added a 2 M Na₂CO₃ solution (0.5 mL). The resultant solution was heated at reflux in an atmosphere of N₂ for 12 h. The reaction mixture was cooled to room temperature and diluted with water and then acidified using 1 N HCl. The aqueous phase was extracted with ethyl acetate (3×10 mL) and the combined organic layer was washed with brine, followed by drying over anhydrous Na₂SO₄. Filtration and removal of the solvent afforded crude product which was further purified by automated prep-HPLC to yield the desired compound. Dark red solid (0.236 g, 73.7%). ¹HNMR (400 MHz, DMSO-d₆): δ10.03 (s, 1H), 7.86-7.81 (m, 2H), 7.77 (d, J=8.3 Hz, 3H), 7.70-7.68 (m 1H), 7.42-7.35 (m, 1H), 7.33-7.30 (m, 1H), 7.18 (d, J=3.6 Hz, 1H), 6.90 (d, J=8.7 Hz, 2H). ¹³C NMR (101 MHz, DMSO-d₆): δ187.4, 159.2, 158.7, 149.4, 145.9, 135.8, 130.9, 126.8, 126.7, 126.3, 126.2, 126.1, 125.0, 123.6, 120.6, 119.3, 116.6, 108.5. LC-MS (ESI) [M+H]⁺: 320.95.

### 5-(Furan-2-yl)-2-hydroxy-N-phenylbenzamide (#21)

To a solution of 5-bromo-2-hydroxybenzoic acid (0.434 g, 1 mmol) in DMF (10 mL) was added HOBt (0.459 g, 3 mmol) and EDC (0.575 g, 3 mmol) and the resulting mixture was stirred at for 30 min. To this mixture aniline (0.279 g, 3 mmol), N,N-diisopropylethylamine (1.05 mL, 6 mmol) were added. The resulting mixture was stirred at for 2 h. The reaction mixture was diluted with ethyl acetate and quenched with the addition of saturated NH₄Cl. The layers were separated, and the aqueous layer was washed three times with ethyl acetate. The combined organic layer was dried over anhydrous Na₂SO₄, concentrated to give the crude product as a light-yellow solid, which was used for the next step without further purifications. White solid (0.569 g, 97.4%). LC-MS (ESI) [M+H]⁺: 292.95. A mixture of 5-bromo-2-hydroxy-N-phenylbenzamide (0.569 g, 1.948 mmol), 2-furanboronic acid 7 (0.326 g, 2.92 mmol), and (Ph₃P)₄Pd (0.0225 g, 0.0.018 mmol) in Dioxane (25 mL) and 2M aqueous Na₂CO₃ (3.9 mL) was flushed with nitrogen for 5 min and heated at 80° C. for 12 h under nitrogen atmosphere. The solvents were removed under reduced pressure, the residue was dissolved in water (1000 mL), the mixture obtained was filtered through celite, and the filtrate was neutralized with 2 N hydrochloric acid. The solids were filtered, washed with water, dried, and recrystallized from ethanol to give 5-(furan-2-yl)-2-hydroxybenzoic acid. Pale yellow solid (0.350 g, 64.3%). ¹H NMR (400 MHz, DMSO-d₆): δ11.87 (s, 1H), 10.49 (s, 1H), 8.25 (d, J=2.2 Hz, 1H), 7.80-7.66 (m, 4H), 7.39 (t, J=7.6 Hz, 2H), 7.15 (t, J=7.5 Hz, 1H), 7.05 (dd, J=8.6, 1.7 Hz, 1H), 6.84 (d, J=3.1 Hz, 1H), 6.62-6.53 (m, 1H). ¹³C NMR (101 MHz, DMSO-d₆): δ166.1, 157.6, 152.6, 142.32, 138.1, 128.8, 124.3, 124.2, 122.1, 122.0, 121.0, 120.8, 118.3, 117.8, 112.0, 104.5. LC-MS (ESI) [M+H]⁺: 279.95.

### (Z)-2-((5-phenylfuran-2-yl)methylene)benzo[b]thiophen-3(2H)-one (#25)

To a mixture of 2-((5-bromofuran-2-yl)methylene)benzo[b]thiophen-3(2H)-one (0.077 g, 0.25 mmol), phenylboronic acid (0.0.37 g, 0.3 mmol) and tetrakistriphenylphosphinepalladium(0) (0.029 g, 0.025 mmol) in DME (5 mL) was added a 2 M Na₂CO₃ solution (0.5 mL). The resulting solution was heated at reflux in an atmosphere of N₂ for 6-12 h. The reaction mixture was cooled to room temperature and diluted with water and then acidified using 1 N HCl. The aqueous phase was extracted with ethyl acetate (3×10 mL) and the combined organic layer was washed with brine, followed by drying over anhydrous Na₂SO₄. Filtration and removal of the solvent afforded crude product that was further purified by automated prep-HPLC to yield the desired compound. Orange solid (0.063 g, 83%). ¹H NMR (400 MHz, CDCl₃): δ 7.90 (d, J=8.7 Hz, 1H), 7.83 (d, J=9.1 Hz, 2H), 7.73 (s, 1H), 7.56-7.53 (m, 2H), 7.47 (t, J=7.8 Hz, 2H), 7.36 (t, J=7.8 Hz, 1H), 7.28-7.26 (m, 1H), 6.94 (d, J=3.7 Hz, 1H), 6.85 (d, J=3.7 Hz, 1H). ¹³CNMR (101 MHz, CDCl₃): δ188.2, 157.6, 150.3, 146.4, 134.8, 130.9, 129.4, 128.9, 128.8, 127.9, 126.8, 125.3, 124.6, 123.9, 120.7, 118.9, 108.9. LC-MS (ESI) [M+H]⁺: 305.

### (Z)-2-Hydroxy-5-(5-((3-oxobenzofuran-2(3H)-ylidene)methyl)furan-2-yl)benzoic acid (#07): Method A

To an ice cooled solution of benzofuranone (0.134 g, 1 mmol) in EtOH (8 mL) was added a solution of NaOH (0.200 g, 5 mmol) in 2 mL water dropwise. To this 5-(5-formylfuran-2-yl)-2-hydroxybenzoic acid (0.116 g, 0.5 mmol) was added and the resulting mixture was gradually warmed to room temperature and stirred for another 1 h. Reaction mixture was then diluted with water and acidified using 1 N HCl. The precipitated product was collected by filtration and washed with water and dried to yield the product as a dark red solid (0.244 g, 70%). Due to the low solubility of the product, only a few milligrams of the crude material were purified by reverse phase HPLC to yield pure material for the biochemical assays. ¹H-NMR ((400 MHz, DMSO-d₆) δ 8.23 (d, J=2.3 Hz, 1H), 7.96 (dd, J=8.7, 2.3 Hz, 1H), 7.76-7.73 (m, 2H), 7.47 (d, J=8.2 Hz, 1H), 7.30-7.26 (m, 3H), 7.18 (d, J=3.7 Hz, 1H), 7.06 (d, J=8.7 Hz, 1H), 6.92 (s, 1H). ¹³C-NMR (101 MHz, DMSO-d₆) δ 183.0, 171.9, 165.4, 161.9, 156.0, 147.9, 144.7, 137.7, 132.0, 126.6, 124.7, 124.4, 122.0, 121.4, 118.7, 113.6, 109.2, 101.4. LC-MS (ESI) [M+H]⁺: 349.

### 2-Hydroxy-5-(5-((2-oxocyclohexylidene)methyl)furan-2-yl)benzoic acid (#14)

This compound was prepared according to Method A using cyclohexanone (0.098 g 1 mmol), NaOH (0.200 g, 5 mmol), and 5-(5-formylfuran-2-yl)-2-hydroxybenzoic acid (0.116 g, 0.5 mmol). Dark red solid (0.126 g, 80.7%). Due to the low solubility of the product, only few milligrams were purified by reverse phase HPLC, yielding pure compound for testing. ¹H NMR (400 MHz, DMSO-d₆): δ8.16 (d, J=2.3 Hz, 1H), 7.93 (dd, J=8.7, 2.4 Hz, 1H), 7.27 (t, J=2.3 Hz, 1H), 7.12 (d, J=3.6 Hz, 1H), 7.08 (d, J=8.7 Hz, 1H), 7.02 (d, J=3.6 Hz, 1H), 2.98-2.88 (m, 2H), 2.43 (t, J=6.3 Hz, 2H), 1.90-1.74 (m, 4H). LC-MS (ESI) [M+H]⁺: 313.

### 2-Hydroxy-5-(5-((1-oxo-1,3-dihydro-2H-inden-2-ylidene)methyl)furan-2-yl)benzoic acid (#09)

This compound was prepared according to method A using 2,3-dihydro-1H-inden-1-one (0.066 g, 0.5 mmol), NaOH (0.080 g, 2 mmol) and 5-(5-formylfuran-2-yl)-2-hydroxybenzoic acid (0.116 g, 0.5 mmol) Red (0.154 g, 93.2%). Due to the low solubility of the product, only few milligrams were purified by reverse phase HPLC, yielding pure compound for testing. Yield represents crude material yield. ¹H-NMR (400 MHz, DMSO-d₆) δ 8.26 (d, J=2.4 Hz, 1H), 8.04 (dd, J=8.7, 2.3 Hz, 1H), 7.95 (s, 1H), 7.77 (d, J=7.6 Hz, 1H), 7.75-7.65 (m, 3H), 7.49 (td, J=7.5, 1.3 Hz, 1H), 7.40 (t, J=2.0 Hz, 1H), 7.21 (q, J=3.7 Hz, 2H), 7.12 (d, J=8.7 Hz, 1H), 4.15 (d, J=2.0 Hz, 2H). LC-MS (ESI) [M+H]⁺: 331.

### (Z)-2-Hydroxy-5-(5-((3-oxobenzo[b]thiophen-2(3H)-ylidene)methyl)thiophen-3-yl)benzoic acid (#10): Method B

To a vigorously stirred solution of LDA (4 mmol) in anhydrous THF (5 mL) at 0° C., a solution of N,N-diethyl-2-(methylthio)benzamide (0.223 g, 1 mmol) and 5-(5-formylfuran-2-yl)-2-hydroxybenzoic acid (0.232 g, 1 mmol) in anhydrous THF (5 mL) was added under nitrogen atmosphere. The resulting mixture was stirred for 2 h, and then gradually warmed to room temperature. The reaction mixture was then poured into H₂O. The pH was adjusted to 4-5 by addition of diluted HCl. The organic layer was separated, and the aqueous layer was extracted with ethyl acetate (3×10 mL). The organic phases were combined and dried over anhydrous Na₂SO₄. Evaporation of the solvent followed by reverse phase HPLC yielded (Z)-2-hydroxy-5-(5-((3-oxobenzo[b]thiophen-2(3H)-ylidene)methyl)thiophen-3-yl)benzoic acid. Red solid (0.222 g, 61%). ¹HNMR (400 MHz, DMSO-d₆): δ8.30 (d, J=2.3 Hz, 1H), 8.02 (dd, J=2.3 Hz, 8.7 Hz, 1H), 7.83 (d, J=7.8 Hz, 1H), 7.74 (s, 1H), 7.72-7.68 (m, 2H), 7.38 (t, J=7.8 Hz, 1H), 7.31 (d, J=3.7 Hz, 1H), 7.24 (d, J=3.7 Hz, 1H), 7.13 (d, J=8.7 Hz, 1H). ¹³CNMR (101 MHz, DMSO-d₆): δ186.9, 171.3, 161.5, 156.5, 149.4, 145.3, 135.5, 131.3, 130.3, 126.5, 126.3, 126.2, 125.9, 124.4, 122.8, 120.5, 118.8, 118.3, 113.9, 109.1. LC-MS (ESI) [M+H]⁺: 365. HRMS (ESI) Calculated for C₂₀H₁₂O₅S [M+H]⁺: 365.0417. Found: 365.0481.

### (Z)-2-Hydroxy-5-(5-((3-oxobenzo[b]thiophen-2(3H)-ylidene)methyl)furan-2-yl)benzonitrile (#05)

This compound was prepared according to Method B using N,N-diethyl-2-(methylthio)benzamide (0.223 g, 1 mmol) and 5-(5-formylfuran-2-yl)-2-hydroxybenzonitrile (0.214 g, 1 mmol). Red solid (0.210 g, 61%). ¹HNMR (400 MHz, DMSO-d₆): δ11.68 (brs, 1H), 8.13 (d, J=1.8 Hz, 1H), 8.01 (dd, J=2.3 Hz, 8.7 Hz, 1H), 7.83-7.81 (m, 2H), 7.79 (s, 1H), 7.71 (t, J=7.3 Hz, 1H), 7.38 (t, J=7.8 Hz, 1H), 7.31 (d, J=3.7 Hz, 1H), 7.26 (d, J=3.7 Hz, 1H), 7.18 (d, J=8.7 Hz, 1H). LC-MS (ESI) [M+H]⁺: 346.

### 2-Hydroxy-5-(5-((2-oxocyclopentylidene)methyl)furan-2-yl)benzoic acid (#06)

This compound was prepared according to Method B using cyclopentanone (0.084 g, 1 mmol), NaOH (0.200 g, 5 mmol), and 5-(5-formylfuran-2-yl)-2-hydroxybenzoic acid (0.116 g, 0.5 mmol). Dark red solid (0.110 g, 73.8%). ¹H NMR (400 MHz, DMSO-d₆): δ8.13 (dd, J=4.1, 2.3 Hz, 1H), 7.92-7.88 (m, 1H), 7.86 (dd, J=6.4, 3.0 Hz, 1H), 7.09 (d, J=3.7 Hz, 1H), 7.04 (d, J=8.7 Hz, 1H), 7.01 (dd, J=5.6, 3.3 Hz, 2H), 6.76 (s, OH), 2.81-2.73 (m, 2H), 2.00-1.92 (m, 2H), 1.92-1.82 (m, 2H). LC-MS (ESI) [M+H]⁺: 299.

### General procedure for the synthesis of amide derivatives: Method C

To a stirred solution of acid (0.2 mmol, 1 equiv.) in DMF (2 mL) at room temperature was added HOBt (0.24 mmol, 1.2 equiv.) in one portion followed by EDC (0.24 mmol, 1.2 equiv.). The resulting mixture was stirred at room temperature for 30 min. To this mixture, amine (0.24 mmol, 1.2 equiv.) and DIEA (0.24 mmol, 1.2 equiv.) were added and stirred for 2 h, After complete consumption of the starting material, the reaction mixture was diluted with water and extracted with EtOAc. The combined organic extracts were washed with water, dried over anhydrous Na₂SO₄ and concentrated under reduced pressure to yield the crude product which was further purified by reverse phase HPLC.

### (Z)—N-(4-Aminophenyl)-2-hydroxy-5-(5-((3-oxobenzo[b]thiophen-2(3H)-ylidene)methyl)furan-2-yl)benzamide (#01)

This compound was prepared according to Method C. Red solid (0.053 g, 58%). ¹H NMR (400 MHz, DMSO-d₆): δ10.22 (s, 1H), 8.41 (s, 1H), 7.94 (d, J=7.3 Hz, 1H), 7.85 (t, J=7.8 Hz, 1H), 7.79 (s, 1H), 7.72-7.67 (m, 2H), 7.40-7.32 (m, 5H), 67.22-7.13 (m, 3H), 6.58 (dd, J=2.2 Hz, 8.7 Hz, 1H). LC-MS (ESI) [M+H]⁺: 455.

### (Z)-2-hydroxy-5-(5-((3-oxobenzo[b]thiophen-2(3H)-ylidene)methyl)furan-2-yl)-N-phenylbenzamide (#02)

This compound was prepared according to Method C. Orange solid (0.065 g, 74%). ¹H NMR (400 MHz, DMSO-d₆): δ10.50 (s, 1H), 8.36 (d, J=2.3 Hz, 1H), 7.87 (dd, J=2.3 Hz, 8.7 Hz, 1H), 7.82-7.75 (m, 3H), 7.75 (s, 1H), 7.71-7.65 (m, 3H), 7.37-7.32 (m, 4H), 7.14 (d, J=3.7 Hz, 1H), 7.08 (t, J=7.3 Hz, 1H), 7.06 (d, J=8.7 Hz, 1H). ¹³CNMR (101 MHz, DMSO-d₆): δ186.9, 165.4, 158.4, 157.1, 149.4, 145.3, 138.3, 135.5, 130.3, 128.9, 128.8, 126.2, 126.1, 126.0, 125.8, 124.5, 124.2, 122.9, 120.7, 120.3, 119.9, 118.8, 118.1, 108.9. LC-MS (ESI) [M+H]⁺: 440. HR-MS (ESI) Calculated for C₂₆H₁₇NO₄S [M+H]⁺: 440.0872. Found: 440.0945.

### (Z)-2-Hydroxy-5-(5-((3-oxobenzo[b]thiophen-2(3H)-ylidene)methyl)furan-2-yl)-N′-phenylbenzohydrazide (#11)

This compound was prepared according to Method C. Red solid (0.052 g, 57%). ¹H NMR (400 MHz, DMSO-d₆): δ12.16 (s, 1H), 10.55 (s, 1H), 8.44 (d, J=2.3 Hz, 1H), 7.87 (d, J=7.8 Hz, 2H), 7.81 (s, 1H), 7.74 (t, J=7.3 Hz, 2H), 7.43 (d, J=7.8 Hz, 1H), 7.37 (d, J=3.7 Hz, 1H), 7.24-7.16 (m, 5H), 6.87 (d, J=8.2 Hz, 1H), 6.76 (t, J=6.9 Hz, 1H). LC-MS (ESI) [M+H]⁺: 455.

### (Z)-2-((5-(4-Hydroxy-3-(piperidine-1-carbonyl)phenyl)furan-2-yl)methylene)benzo[b]thiophen-3(2H)-one (#12)

This compound was prepared according to Method C. Reddish orange solid (0.037 g, 43%). ¹HNMR (400 MHz, DMSO-d₆): δ8.36 (s, 1H), 7.78 (t, J=7.8 Hz, 2H), 7.72 (d, J=8.2 Hz, 1H), 7.71 (s, 1H), 7.67 (t, J=7.3 Hz, 1H), 7.59 (s, 1H), 7.33 (t, J=7.3 Hz, 1H), 7.27 (d, J=3.7 Hz, 1H), 7.14 (d, J=3.7 Hz, 1H), 7.01 (d, J=8.2 Hz, 1H), 3.62 (brs, 4H), 1.52 (brs, 2H), 1.48 (brs, 4H). LC-MS (ESI) [M+H]⁺: 432.

### (Z)-2-Hydroxy-N-isopropyl-5-(5-((3-oxobenzo[b]thiophen-2(3H)-ylidene)methyl)furan-2-yl)benzamide (#13)

This compound was prepared according to Method C. Red solid (0.047 g, 58%). ¹H NMR (400 MHz, DMSO-d₆): δ8.70 (d, J=4.1 Hz, 1H), 8.37 (s, 1H), 7.92 (d, J=8.2 Hz, 1H), 7.85-7.71 (m, 4H), 7.41-7.39 (m, 2H), 7.17-7.10 (m, 2H), 4.17 (m, 1H), 1.24 (d, J=6.4 Hz, 6H). ¹³C NMR (101 MHz, DMSO-d₆): δ186.9, 167.3, 160.6, 157.0, 149.3, 145.3, 133.5, 130.2, 129.1, 126.3, 126.1, 125.8, 124.8, 124.4, 122.7, 119.9, 118.7, 118.3, 116.3, 108.8, 41.2, 22.1. LC-MS (ESI) [M+H]⁺: 406.

### (Z)-3-(5-((3-Oxobenzo[b]thiophen-2(3H)-ylidene)methyl)furan-2-yl)-N-phenylbenzamide (#15)

This compound was prepared according to Method C. Orange solid (0.056 g, 66%). ¹H NMR (400 MHz, DMSO-d₆): δ10.41 (s, 1H), 8.38 (s, 1H), 8.07 (d, J=7.8 Hz, 1H), 7.92 (d, J=7.8 Hz, 1H), 7.81 (d, J=7.8 Hz, 1H), 7.78-7.75 (m, 4H), 7.69 (t, J=7.8 Hz, 2H), 7.42 (d, J=3.7 Hz, 1H), 7.36-7.32 (m, 4H), 7.09 (t, J=8.7 Hz, 1H). ¹³C NMR (101 MHz, DMSO-d₆): δ 187.0, 165.1, 156.2, 150.2, 145.3, 139.0, 136.1, 135.7, 130.1, 129.5, 129.1, 128.6, 127.9, 127.2, 126.8, 126.3, 125.9, 124.5, 123.8, 123.7, 122.3, 120.4, 118.6, 110.9. LC-MS (ESI) [M+H]⁺: 424.

### (Z)—N-Benzyl-3-(5-((3-oxobenzo[b]thiophen-2(3H)-ylidene)methyl)furan-2-yl)benzamide (#16)

This compound was prepared according to Method C. Reddisch orange solid (0.055 g 63%). ¹HNMR (400 MHz, DMSO-d₆): δ9.59 (brs, 1H), 8.42 (d, J=1.8 Hz, 1H), 7.92 (dd, J=1.8 Hz, 8.2 Hz, 1H), 7.83 (d, J=7.8 Hz, 1H), 7.77 (s, 1H), 7.76 (d, J=7.8 Hz, 1H), 7.71 (t, J=8.4 Hz, 1H), 7.39-7.32 (m, 8H), 7.12 (d, J=3.7 Hz, 1H), 7.08 (d, J=8.7 Hz, 1H), 4.57 (d, J=5.5 Hz, 2H). ¹³CNMR (101 MHz, DMSO-d₆): δ186.8, 167.9, 156.3, 149.3, 145.3, 138.9, 135.4, 130.3, 129.2, 128.4, 127.3, 126.9, 126.2, 125.8, 125.1, 124.4, 122.8, 118.7, 118.6, 116.7, 108.6, 42.4. LC-MS (ESI) [M+H]⁺: 438.

### (Z)-2-Methoxy-5-(5-((3-oxobenzo[b]thiophen-2(3H)-ylidene)methyl)furan-2-yl)-N-phenylbenzamide (#18)

This compound was prepared according to Method C. Reddish orange solid (0.053 g, 58%). ¹HNMR (400 MHz, DMSO-d₆): δ10.29 (s, 1H), 8.12 (d, J=1.8 Hz, 1H), 8.05 (dd, J=1.8 Hz, 8.7 Hz, 1H), 7.86 (d, J=7.8 Hz, 1H), 7.80 (s, 1H), 7.78-7.70 (m, 4H), 7.42-7.34 (m, 6H), 7.13 (t, J=6.9 Hz, 1H), 3.97 (s, 3H). ¹³CNMR (101 MHz, DMSO-d₆): δ186.9, 164.0, 156.9, 156.6, 145.5, 145.2, 139.0, 135.5, 130.3, 128.7, 127.6, 126.5, 126.3, 125.9, 125.7, 124.5, 123.6, 122.9, 121.5, 119.6, 118.8, 112.9, 109.4, 56.2. LC-MS (ESI) [M+H]⁺: 454.

### (Z)-2-((5-(4-Hydroxy-3-(piperazine-1-carbonyl)phenyl)furan-2-yl)methylene)benzo[b]thiophen-3(2H)-one (#19)

This compound was prepared according to Method C. Reddish orange solid (0.046 g, 53%). ¹HNMR (400 MHz, DMSO-d₆): δ8.18 (s, 1H), 8.01 (s, 1H), 7.80-7.74 (m, 3H), 7.72 (s, 1H), 7.68 (d, J=7.3 Hz, 1H), 7.76 (d, J=1.8 Hz, 1H), 7.36 (t, J=7.3 Hz, 1H), 7.29 (d, J=3.7 Hz, 1H), 7.17 (d, J=3.7 Hz, 1H), 7.03 (d, J=8.2 Hz, 1H), 3.36-3.32 (m, 4H), 3.28-3.25 (m, 4H). ¹³C NMR (101 MHz, DMSO-d₆): δ187.3, 166.7, 157.9, 155.1, 149.6, 145.1, 135.9, 130.8, 127.0, 126.7, 125.4, 125.3, 125.0, 123.5, 120.7, 119.3, 117.1, 109.2, 45.9, 44.8. LC-MS (ESI) [M+H]⁺: 433.

### (Z)—N-Isopropyl-3-(5-((3-oxobenzo[b]thiophen-2(3H)-ylidene)methyl)furan-2-yl)benzamide (#20)

This compound was prepared according to Method C. Orange solid (0.049 g, 63%). ¹H NMR (400 MHz, DMSO-d₆): δ8.33 (d, J=7.3 Hz, 1H), 8.27 (s, 1H), 7.99 (d, J=7.8 Hz, 11H), 7.81 (d, J=7.8 Hz, 2H), 7.78 (s, 11H), 7.76 (d, J=7.8 Hz, 1H), 7.69 (t, J=7.3 Hz, 1H), 7.69 (t, J=7.3 Hz, 11H), 7.38-7.33 (m, 3H), 4.12-4.07 (m, 11H), 1.14 (d, J=6.4 Hz, 6H). ¹³C NMR (101 MHz, DMSO-d₆): δ187.0, 164.8, 156.4, 150.1, 145.3, 139.9, 135.7, 130.1, 129.2, 128.8, 127.7, 127.1, 126.3, 125.9, 124.5, 123.4, 122.4, 118.7, 110.7, 41.1, 22.3. LC-MS (ESI) [M+H]⁺: 390.

### (Z)-2-Hydroxy-N-(2-hydroxyethyl)-5-(5-((3-oxobenzo[b]thiophen-2(3H)-ylidene)methyl)furan-2-yl)benzamide (#22)

This compound was prepared according to Method C. Reddish orange solid (0.036 g, 44%). 1H NMR (400 MHz, DMSO-d₆): δ8.99 (t, J=5.6 Hz, 1H), 8.43 (d, J=2.3 Hz, 1H), 7.94 (dd, J=8.6, 2.2 Hz, 1H), 7.85 (dd, J=7.9, 1.3 Hz, 1H), 7.84-7.81 (m, 1H), 7.80 (s, 1H), 7.75-7.70 (m, 11H), 7.40 (td, J=7.4, 1.0 Hz, 11H), 7.37 (d, J=3.8 Hz, 11H), 7.17 (d, J=3.7 Hz, 11H), 7.12 (d, J=8.6 Hz, 1H), 4.88 (s, 1H), 3.60-3.57 (m, 2H), 3.45-3.41 (m, 2H). LC-MS (ESI) [M+H]⁺: 408.

### (Z)-2-((5-(4-Hydroxy-3-(4-methylpiperazine-1-carbonyl)phenyl)furan-2-yl)methylene)benzo[b]thiophen-3(2H)-one (#23)

This compound was prepared according to Method C. Reddish orange solid (0.052 g, 58%). ¹HNMR (400 MHz, DMSO-d₆): δ7.86-7.68 (m, 6H), 7.40 (t, J=7.3 Hz, 1H), 7.34 (d, J=3.7 Hz, 1H), 7.23 (d, J=3.7 Hz, 1H), 7.02 (d, J=8.2 Hz, 1H), 3.43 (brs, 4H), 2.31 (brs, 4H), 2.19 (s, 3H). LC-MS (ESI) [M+H]⁺: 447.

### (Z)-2-Fluoro-5-(5-((3-oxobenzo[b]thiophen-2(3H)-ylidene)methyl)furan-2-yl)-N-phenylbenzamide (#24)

This compound was prepared according to Method C. Reddish orange solid (0.052 g, 59%). ¹HNMR (400 MHz, DMSO-d₆): δ10.61 (s, 1H), 8.43 (s, 1H), 8.20 (dd, J=2.8 Hz, 7.6 Hz, 1H), 7.86 (d, J=7.8 Hz, 1H), 7.80 (s, 1H), 7.78-7.70 (m, 5H), 7.59 (t, J=9.2 Hz, 1H), 7.45 (d, J=3.7 Hz, 1H), 7.43-7.34 (m, 3H), 7.15 (t, J=7.3 Hz, 1H). LC-MS (ESI) [M+H]⁺: 442.

### Protein Expression and Purification

Recombinant human full-length SHP2 (1-594) WT and E76K mutant, as well as the SHP2 catalytic domain (248-527) were expressed and purified as described before (36,41). Recombinant human PTP1B (1-300) and the codon optimized STEP (280-566) catalytic domains were cloned into PET-15b and expressed as N-His tagged fusion proteins. For expression, transformed BL21(DE3) cells were grown and induced similar as described for SHP2. Collected cells were resuspended in lysis buffer (25 mM Tris pH 7.5, 300 mMNaCl, 50 mM imidazole, 10% glycerol) with 100 mg/L RNaseA and were lysed with two passages using a microfluidizer. The lysate was clarified by centrifugation at 15,000×g for 50 min and applied to Ni-NTA resin. The column resin was washed, and the PTP1B or STEP protein was eluted in lysis buffer at 300 mM imidazole. The PTP1B or STEP protein was further purified by S75 size exclusion chromatography in 50 mM Tris, pH 7.5, 50 mMNaCl. The eluted peak fractions were supplemented with TCEP to 10 mM, concentrated by ultrafiltration, and stored at −80° C. The purified yield of PTP1B was 33 mg/L cell culture; the yield of STEP was 16 mg/L cell culture.

### SHP2, PTP1B, and STEP Biochemical Inhibition Assays

SHP2 inhibitors were tested at room temperature (RT) in a 384-well plate format standard phosphatase fluorescence intensity assay using DiFMUP (Invitrogen/Thermo Fisher Scientific, Carlsbad, CA) as a substrate and a total reaction volume of 25 μL. SHP2 inhibitors or vehicle (DMSO) were spotted in triplicate into a black Greiner FLUOTRAC™ 200 384-well microplate (#781076, Greiner) for a 10-point dose-response assay using an Echo®555 Liquid Handler (Labcyte, Inc., San Jose, CA). PTP working solutions were prepared at a 0.625 nM concentration (for a final concentration of 0.5 nM) in buffer containing 50 mM Bis-Tris pH 6.0, 50 mM NaCl, 5 mM DTT, and 0.01% Tween® 20. Prior to the assay, a dually phosphorylated insulin receptor substrate 1 (IRS-1) peptide [625 nM (500 nMfinal) (30)] was added to full-length WT SHP2 working solutions and incubated for 20 min. DiFMUP working solutions at 5× final concentration were prepared in 50 mM Bis-Tris pH 6.0, 50 mMNaCl, and 0.01% Tween 20. 20 μL of PTP working solution was dispensed into the microplate and incubated with inhibitor for 20 min at RT. 5X DiFMUP working solutions were prepared for final concentrations corresponding to the respective Kₘ value for each protein (SHP2-WT, 60 μM; SHP2-E76K, 20 μM; SHP2cat, 20 μM; PTP1B, 25 μM; STEP, 4 μM). The reaction was initiated by addition of 5 μL DiFMUP working solutions. Fluorescence intensity was measured in kinetic mode (every minute for 7 or 10 min) using a Tecan Spark® Multimode Microplate Reader (Tecan, Groedig, Austria) with an excitation wavelength of 360 nm and an emission wavelength of 460 nm. The initial rates were determined from the linear progression curves of the PTP reaction. The nonenzymatic hydrolysis of the substrate was corrected by using a control without addition of enzyme. IC₅₀ values were calculated from the corrected initial rates by nonlinear regression using the program GraphPad Prism (GraphPad Software, Inc., San Diego, CA) Version 8. Dose-response inhibition assays of SHP2cat using the substrate OMFP were performed similarly as described above for DiFMUP. OMFP was used at a concentration corresponding to its Kₘ value for SHP2cat (50 μM final). Fluorescence intensity was measured in kinetic mode (every minute for 10 min) using a Tecan Spark Multimode Microplate Reader with an excitation wavelength of 485 nm and an emission wavelength of 535 nm. Initial rates were determined and IC₅₀ vales calculated as described above.

### Michaelis-Menten Kinetics and Mode of Inhibition Determination

#02 was tested with SHP2-E76K using a similar assay format as described above. #02 or vehicle (DMSO) were spotted in triplicate into a black Greiner FLUOTRAC™ 200 384-well microplate (#781076, Greiner) using an Echo 555 Liquid Handler (Labcyte, Inc., San Jose, CA). SHP2-E76K working solution was prepared and dispensed as described above and incubated with inhibitor for 20 min at RT. DiFMUP working solutions at 5×final concentration were prepared in 50 mM Bis-Tris pH 6.0, 50 mMNaCl, and 0.01% Tween 20. The reaction was initiated by addition of 5 μL DiFMUP working solutions for final DiFMUP concentrations of 100, 50, 25, 12.5, 6.25, 3.125 μM. Fluorescence intensity was measured in kinetic mode as described above. The initial rates were determined from the linear progression curves of each SHP2-E76K reaction. The nonenzymatic hydrolysis of the substrate was corrected for each DiFMUP concentration by using a control without addition of enzyme. Michaelis-Menten plots were generated for each inhibitor concentration using nonlinear regression and fitting initial rates to the Michaelis-Menten equations for competitive, noncompetitive, uncompetitive, or mixed inhibition using the program GraphPad Prism Version 8. For a comparison of the fitting results, the second-order corrected Akaike's Information Criterion (AICc) was calculated using equation 1, where N is the number of data points, SS the absolute sum of squares, and K the number of parameters fit by nonlinear regression plus 1.

$$AICc = Nln ( SS / N ) + 2 K + ( 2 K ( K + 1 ) ) / ( N - K - 1 ) \qquad [ Equation 1 ]$$

The probability for one mode of inhibition compared to another one was computed by using equation [2], where A is the difference between AICc scores of the two models being compared.

$$probability = exp ( - 0.5 Δ ) / ( 1 + exp ( - 0.5 Δ ) ) \qquad [ Equation 2 ]$$

### Jump Dilution Inhibition Assay

#01 was tested for reversibility of inhibition in a jump-dilution experiment using SHP2cat. A regular dose-response experiment (no jump-dilution) was performed in parallel. Working solutions of #01 in DMSO included 10, 3, 1, 0.3, 0.1, 0.03, 0.01, 0 mM for the jump-dilution experiment, and 1, 0.3, 0.1, 0.03, 0.01, 0.003, 0.001, 0 mM for the regular dose-response experiment. SHP2cat working solutions were prepared at concentrations of 6.25 nM for the jump-dilution experiment, and 0.625 nM for the regular dose-response experiment, in buffer containing 50 mM Bis-Tris pH 6.0, 50 mM NaCl, 0.5 mM EDTA, 5 mM DTT, and 0.01% Tween® 20. Separate tubes were prepared for each experimental condition, containing 99 μL of the respective SHP2cat working solution and 1 μL compound solution. SHP2cat and compound were incubated for 10 min at RT, before 900 μL buffer was added to the jump-dilution experiment tubes, resulting in a 10× dilution. SHP2cat/compound mixtures were incubated for another 10 min at RT, before 20 μL of each solution was transferred into a black Greiner FLUOTRAC™ 200 384-well microplate (#781076, Greiner) for a quadruplicate measurement. The enzyme reaction was started by the addition of 5 μL of DiFMUP working solution (100 μM in 50 mM Bis-Tris pH 6.0, 50 mMNaCl, 0.5 mM EDTA, and 0.01% Tween® 20), and fluorescence intensity was measured in kinetic mode (every minute for 10 min) using a Tecan Spark® Multimode Microplate Reader as described above. The final concentrations were as follows: SHP2cat, 0.5 nM; DiFMUP, 20 μM; #01; 8, 2.4, 0.8, 0.24, 0.08, 0.024, 0.008, 0 μM. The initial rates were determined from the linear progression curves of the SHP2cat reaction. The nonenzymatic hydrolysis of the substrate was corrected by using a control without addition of enzyme. IC₅₀ values were calculated from the corrected initial rates by nonlinear regression using the program GraphPad Prism (GraphPad Software, Inc., San Diego, CA) version 9.

### Protein Thermal Shift Assay (PTS)

Differential scanning fluorimetry (also known as protein thermal shift) measurements of SHP2cat were performed. #02 was spotted at different concentrations into MicroAmp®384-well real-time PCR plates (#4483285, Applied Biosystems, Waltham, MA) using an Echo 555 Liquid Handler. 5 μL of SHP2cat working solution (1.5 μM in 50 mM Tris-HCl pH 7.5, 50 mM NaCl, and 5 mM DTT) was added to each well using a Multidrop™ Combi Reagent Dispenser (Thermo Fisher Scientific, Waltham, MA). 5 μL of 5×SYPRO Orange (Invitrogen/Thermo Fisher Scientific, Carlsbad, CA) dissolved in molecular grade water was equally dispensed into the PCR plate wells, diluting the enzyme solution 1:2. The plate was then sealed with MicroAmp Optical Adhesive Film (Applied Biosystems, Waltham, MA) and spun to collect the reaction mix at the bottom of the plate. Plates were analyzed using a ViiA® 7 Real-Time PCR instrument (Applied Biosystems, Waltham, MA) and a 12 min temperature gradient with a temperature increase of 0.075° C./s. The melting temperature and thermal profiles were determined using Protein Thermal Shift™ Software v1.3 (Applied Biosystems, Waltham, MA).

### Cell Culture

The acute myeloid leukemia (AML) cell lines MOLM-13 and Kasumi-1 and the esophageal carcinoma cell line KYSE-520 were obtained from DSMZ (German Collection of Microorganisms and Cell Cultures). AML cell lines MV-4-11 and U937 were obtained from ATCC. Cell lines were cultured in RPMI 1640 media with L-Glutamine (Coming) supplemented with 10% FBS (Gibco) and 1% Antibiotic-Antimycotic solution (Gibco). Cells were incubated in flasks at 37° C. and 5% CO₂.

Patient-derived AML cells were obtained from Carol Burian and Dr. James Mason (Scripps MD Anderson, La Jolla, CA) under approved IRB protocol 13-6180. Peripheral blood mononuclear cells (PBMCs) were isolated by traditional Ficoll-Paque™ PLUS (17-1440-02, GE Healthcare) centrifugation according to the manufacturer's instructions, and red blood cells were lysed using RBC Lysis Buffer (Alfa Aesar, cat. #J62990). Final PBMCs were resuspended in Bambanker serum-free freezing medium (Wako Pure Chemical Industries, Ltd.) and stored frozen before cell viability was assessed as described below.

### Cell Viability Assays

Viability of AML, esophageal carcinoma, and TF-1 cells was assessed using the ATP-depletion assay CellTiter-Glo® (Promega). Cells were harvested at 1×10⁶/mL to 2×10⁶ cells/mL with cell numbers determined by trypan blue using the Countess Cell counter (Thermo Scientific) and resuspended in culture media. 3K cells (in 20 mL) were seeded in a 384-well format white, clear bottom microplate (#781098, Greiner) spotted with test compound or vehicle control (DMSO) using an Echo 555 Liquid Handler. Cells were incubated for 3 days, before 10 mL of CellTiter-Glo reagent mix was added to each well and incubated for 10 min at RT. Luminescence was read on a Tecan Spark® Multimode Microplate Reader and data analyzed using Graph Pad Prism software.

Triple-negative breast cancer cell viability was assessed using the ATP-depletion assay CellTiter-Glo (Promega). Briefly, 2.5K cells in 25 mL media (RPMI+10% FBS+1× Penicillin-Streptomycin (Omega Scientific)) were seeded per well of a 384-well tissue culture treated plate (Greiner). 25 nL of 1000× test compound were added using a Labcyte Echo acoustic dispenser and the cells incubated for 5 days before addition of 10 mL of CellTiter-Glo reagent as described by the manufacturer. Luminescence was detected on a BioTek Synergy 2 microplate reader and the values normalized to those of vehicle (DMSO) treated controls before being plotted using GraphPad Prism.

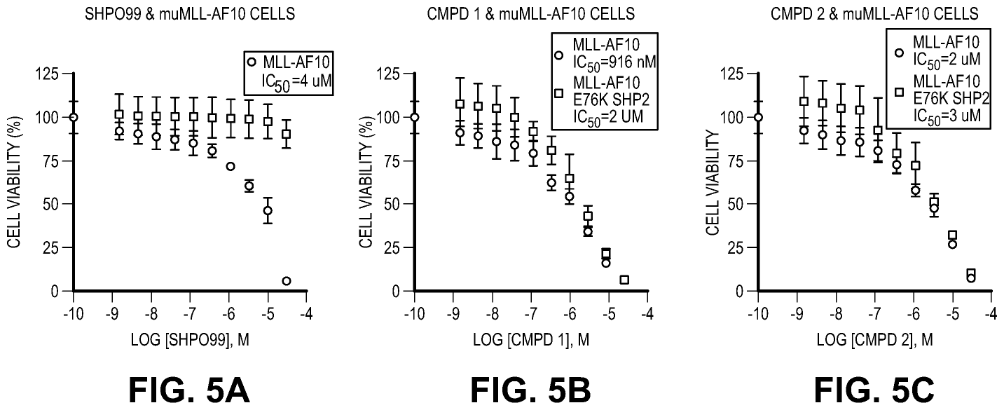

Cell viability assays of SHP099, #01, and #02 against mouse MLL-AF10 leukemia cells were conducted. Two MLL-AF10 cell lines were employed, one expressing wild-type SHP2, and the other expressing E76K mutant SHP2. Cell viability data from these experiments are presented in FIG. 5. While the IC₅₀ of SHP099 against the wild-type MLL-AF10 cells was found to be 4 μM, the activity of SHP099 against the E76K mutant MLL-AF10 cells was insufficient for an IC₅₀ value to be determined. In contrast, #01 displayed measurable activity against both lines of MLL-AF10 cells: the IC₅₀ of #01 against the wild-type MLL-AF10 cells was found to be 916 nM, and the IC₅₀ of #01 against the E76K mutant MLL-AF10 cells was found to be 2 μM. Further, #02 displayed measurable activity against both lines of MLL-AF10 cells: the IC₅₀ of #02 against the wild-type MLL-AF10 cells was found to be 2 μM, and the IC₅₀ of #02 against the E76K mutant MLL-AF10 cells was found to be 3 μM.

### Cell Colony Formation Assays

MDA-MB-468 and BT-459 triple-negative breast cancer cells were maintained in DMEM+10% FBS supplemented with 1× Penicillin-Streptomycin/L-Glutamine (Omega Scientific). 1.5K cells (in 300 mL) were seeded per well of a standard 24-well tissue culture plate (Falcon) and allowed to adhere for 24 h, before the addition of drug(s) as described. After a further 96 h, media was replaced with fresh media containing the same test compounds and concentrations. After 11 days, media was removed and cells stained with 0.5% Crystal Violet (in 20% methanol) for 20 min with agitation @ 80 rpm. Stain was removed and plates with stained cells were washed by being submerged in excess deionized water and allowed to air dry overnight before imaging.

### Immunoblot Assays

Cells cultured in RPMI medium were treated as indicated in figure legends. Total protein extracts were prepared in modified RIPA lysis buffer (25 mM Tris-HCl, pH 7.4, 10% glycerol, 0.2% Triton™ X-100, 150 mM NaCl, 2 mM Na3VO4, 1 mM EDTA) containing a protease inhibitor cocktail (Roche Applied Science, Indianapolis, IN). Equal amounts of protein were separated on 4-12% (p-ERK1/2 blots) or 4-20% (SHP2 blots) Bis-Tris gels by SDS-PAGE and transferred to nitrocellulose (p-ERK1/2 blots) or 0.2 μm PVDF (SHP2 blots) membranes. Membranes were blocked in 5% dry milk (Bio-Rad, Hercules, CA) in TBS-Tween 20 (0.1% vol/vol; TBS-T) for 1 h at room temperature. Protein-bound membranes were incubated with indicated primary antibody overnight at 4° C. After washing three times with TBS-T, membranes were incubated for 1 h with horseradish peroxidase-conjugated secondary antibody, then visualized by an ECL Prime detection system (GE Healthcare). Immunoblotting experiments were carried out at least three times and representative images are shown. Densitometric quantitation of immunoblots was performed using Image Studio Lite Software. Changes of phosphorylated ERK1/2 to total ERK1/2 rations are expressed as percentage of the vehicle-treated controls. The following antibodies were used: Phospho-Erk1/2 Ab, Cell Signaling, #9101S; total Erk1/2 Ab, Cell Signaling, #9102S; SHP2 Ab, Bethyl, A301-544A, lot #1; GAPDH (14C10) mAb, Cell Signaling, #2118S, lot #14.

### SHP2 CRISPR-Cas9 Knockout and Inhibitor Selectivity Assays

High-efficiency Cas9-editing MOLM-13 cells were generated by transducing MOLM-13 cells with the pLenti-Cas9-blasticidin construct (Addgene plasmid #52962) and selecting single stable clones using flow-sorting. Clones were then tested for editing efficiency by performing TIDE analysis (45). These MOLM13-Cas9 cells were then transduced with a lentiviral construct containing an AAVS sgRNA and an mCherry reporter out of frame and downstream of an AAVS sgRNA targeting site. The cells were bulk-sorted for mCherry+expression, indicative of successful AAVS editing, and used in subsequent experiments.

For SHP2 KO, dual sgRNAs targeting SHP2 were cloned in the pLentiGuide puro vector (Addgene plasmid #52963) using a published protocol (50). This SHP2 dual sgRNA plasmid was used to make virus using standard protocols with pPAX2 and pMD.2 as packaging vectors. The virus was used to stably transduce MOLM13-Cas9-mCherry cells, and cells were selected using puromycin (1 μg/mL). SHP2 KO was evaluated by immunoblotting. SHP2 inhibitors were tested in parallel in regular MOLM-13 cells expressing SHP2, and in MOLM-13-Cas9-mCherry cells with SHP2 KO. Experiments with regular MOLM-13 cells were performed as described above. For SHP2 KO, MOLM-13-Cas9-mCherry cells were seeded in nontreated 6-well-plates (2×10≢cells/well in 2.5 mL), with cell numbers determined by trypan blue using the Countess Cell counter (Thermo Scientific). Cells were transduced with 10 μL of SHP2 dual sgRNA lentivirus added to the culture media. Polybrene (Sigma-Aldrich, 10 mg/mL, TR-1003-G) at a concentration of 0.8 μg/mL was added to experimental wells. Cells were incubated overnight at standard cell culture conditions, before spun down and resuspended in fresh media (RPMI 1640 with L-Glutamine+10% FBS+1% Antibiotic-Antimycotic solution+1% L-Glutamine solution). Puromycin (Gibco, 10 mg/mL) at a concentration of 2.5 μg/mL was added to the cells 48 h after transduction for 3 d. 3K cells/well (in 20 μL) were seeded in a 384-well white, clear bottom microplate (#781098, Greiner) and spotted with test compounds or vehicle control (DMSO) using an Echo 555 Liquid Handler. Cells were incubated for 3 days at standard cell culture conditions. Cell viability was assessed using the ATP-depletion assay CellTiter-Glo® (Promega) as described above, and data were analyzed using GraphPad Prism software.

### Pharmaceutical Compositions

### Example A-1: Parenteral Pharmaceutical Composition

To prepare a parenteral pharmaceutical composition suitable for administration by injection (subcutaneous, intravenous), 1-1000 mg of a water-soluble salt of a compound described herein, or a pharmaceutically acceptable salt or solvate thereof, is dissolved in sterile water and then mixed with 10 mL of 0.9% sterile saline. A suitable buffer is optionally added as well as optional acid or base to adjust the pH. The mixture is incorporated into a dosage unit form suitable for administration by injection.

### Example A-2: Oral Solution

To prepare a pharmaceutical composition for oral delivery, a sufficient amount of a compound described herein, or a pharmaceutically acceptable salt thereof, is added to water (with optional solubilizer(s), optional buffer(s) and taste masking excipients) to provide a 20 mg/mL

### Solution

### Example A-3: Oral Tablet

A tablet is prepared by mixing 20-50% by weight of a compound described herein, or a pharmaceutically acceptable salt thereof, 20-50% by weight of microcrystalline cellulose, 1-10% by weight of low-substituted hydroxypropyl cellulose, and 1-10% by weight of magnesium stearate or other appropriate excipients. Tablets are prepared by direct compression. The total weight of the compressed tablets is maintained at 100-500 mg.

### Example A-4: Oral Capsule

To prepare a pharmaceutical composition for oral delivery, 1-1000 mg of a compound described herein, or a pharmaceutically acceptable salt thereof, is mixed with starch or other suitable powder blend. The mixture is incorporated into an oral dosage unit such as a hard gelatin capsule, which is suitable for oral administration.

In another embodiment, 1-1000 mg of a compound described herein, or a pharmaceutically acceptable salt thereof, is placed into Size 4 capsule, or size 1 capsule (hypromellose or hard gelatin) and the capsule is closed.

### Example A-5: Topical Gel Composition

To prepare a pharmaceutical topical gel composition, a compound described herein, or a pharmaceutically acceptable salt thereof, is mixed with hydroxypropyl celluose, propylene glycol, isopropyl myristate and purified alcohol USP. The resulting gel mixture is then incorporated into containers, such as tubes, which are suitable for topical administration.

While preferred embodiments have been shown and described herein, it will be understood by those skilled in the art that such embodiments are provided by way of example only. It is not intended that the embodiments be limited by the specific examples provided within the specification. Numerous variations, changes, and substitutions will now occur to those skilled in the art without departing from the embodiments. It should be understood that various alternatives to the embodiments described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

What is claimed is:

1. A compound of Formula (I): 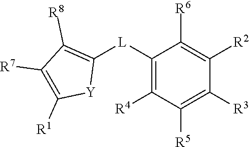
or a pharmaceutically acceptable salt thereof, wherein,
R¹ is: 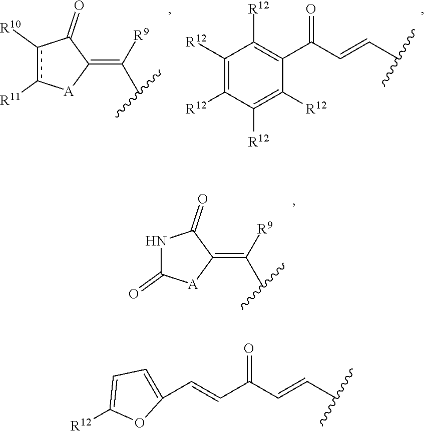
or hydrogen;
R² is —H, halogen, —OH, —C(O)N(R¹²)₂, —CN, —COOH, —C(O)NR¹²N(R¹²)₂, —C(O)R¹², 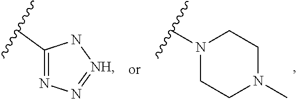
A is —CH₂—, —S—, —O—, —CH₂—CH₂—, —C(R¹²)₂—, or —C(R¹²)₂—C(R¹²)₂—;
Y is —O—, —S—, —NH— or —CH═CH—;
L is a bond, —CH═, or —C(═O)CH═CH—.
R³ is hydrogen, —OH, —F, —Cl, —CF₃, —OCF₃, or —Br;
R⁴, R⁵, R⁶, R⁷, R⁸, and R⁹ are each independently hydrogen, halogen, —OR¹³, —SR¹³, —N(R¹³)₂, —NO₂, —C(O)OR¹³, —C(O)N(R¹³)₂, —C(S)OR¹³, —C(S)SR¹³, —C(O)SR¹³, C₁-C₁₆ substituted or unsubstituted alkyl, C₁-C₁₆ substituted or unsubstituted alkenyl, substituted or unsubstituted C₆ aryl, substituted or unsubstituted C₃-C₈ cycloalkyl, substituted or unsubstituted C₃-C₈ cycloalkenyl, substituted or unsubstituted C₃-C₈ cycloalkynyl, substituted or unsubstituted heterocycloalkyl, substituted or unsubstituted heterocycloalkenyl, substituted or unsubstituted heterocycloalkynyl, or C₁-C₁₆ substituted or unsubstituted alkynyl;
R¹⁰ and R¹¹ either together form a substituted or unsubstituted 6 membered aryl ring, or are each independently hydrogen, —OR¹³, —SR¹³, —N(R¹³)₂, —NO₂, —CN, —C(O)OR¹³, —C(O)N(R¹³)₂, —C(S)OR¹³, —C(S)SR¹³, —C(O)SR¹³, C₁-C₁₆ substituted or unsubstituted alkyl, C₁-C₁₆ substituted or unsubstituted alkenyl, substituted or unsubstituted C₆ aryl, substituted or unsubstituted C₃-C₈ cycloalkyl, substituted or unsubstituted C₃-C₈ cycloalkenyl, substituted or unsubstituted C₃-C₈ cycloalkynyl, substituted or unsubstituted heterocycloalkyl, substituted or unsubstituted heterocycloalkenyl, substituted or unsubstituted heterocycloalkynyl, or C₁-C₁₆ substituted or unsubstituted alkynyl;
R¹² are each independently hydrogen, halogens, —OH, —CN, —SH, —NO₂, 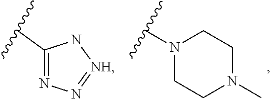
C₁-C₁₆ substituted or unsubstituted alkyl, C₁-C₁₆ substituted or unsubstituted alkyl, C₁-C₁₆ substituted or unsubstituted alkenyl, substituted or unsubstituted C₆ aryl, substituted or unsubstituted C₃-C₈ cycloalkyl, substituted or unsubstituted C₃-C₈ cycloalkenyl, substituted or unsubstituted C₃-C₈ cycloalkynyl, substituted or unsubstituted heterocycloalkyl, substituted or unsubstituted heterocycloalkenyl, substituted or unsubstituted heterocycloalkynyl, or C₁-C₁₆ substituted or unsubstituted alkynyl;
R¹³ are each independently C₁-C₁₆ substituted or unsubstituted alkyl, C₁-C₁₆ substituted or unsubstituted alkyl, C₁-C₁₆ substituted or unsubstituted alkenyl, substituted or unsubstituted C₆ aryl, substituted or unsubstituted C₃-C₈ cycloalkyl, substituted or unsubstituted C₃-C₈ cycloalkenyl, substituted or unsubstituted C₃-C₈ cycloalkynyl, substituted or unsubstituted heterocycloalkyl, substituted or unsubstituted heterocycloalkenyl, substituted or unsubstituted heterocycloalkynyl, or C₁-C₁₆ substituted or unsubstituted alkynyl;
wherein
when R¹ is hydrogen, R² is 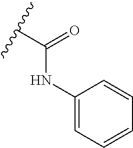 [SMILES: *C(=O)Nc1ccccc1]
and R³ is —OH;
when A is —C(R¹²)₂—C(R¹²)₂—, R² is —C(O)OH and R³ is —OH or R³ is —C(O)OH and R² is —OH;
with the proviso that the compound is not a molecule from Table 1.

2. The compound of claim 1 wherein R¹ is

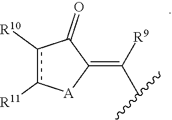

3. The compound of claim 2, wherein R⁹ is hydrogen.

4. The compound of claim 2, wherein A is —O—, —S—, —CH₂—, or —CH₂CH₂—.

5. The compound of claim 1, wherein R¹ is

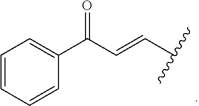 [SMILES: */C=C/C(=O)c1ccccc1]

6. The compound of claim 5 or any one of claims 1-5, wherein R³ is —OH or a halogen.

7. The compound of claim 5 or any one of claims 1-5, wherein R² is —C(═O)—N(R¹²)₂, or —C(═O)—OR¹².

8. The compound of claim 1, wherein R¹ is H.

9. The compound of claim 1, wherein R³ is —OH or a halogen.

10. The compound of claim 1, wherein R² is —C(═O)N(R²)₂.

11. The compound of claim 6, wherein R³ is —OH.

12. The compound of claim 6, wherein R² is —C═O—NHR¹².

13. The compound of claim 12, wherein R² is

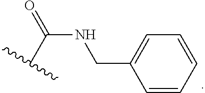 [SMILES: *C(=O)NCc1ccccc1]

14. The compound of claim 4 or any one of claims 1-4, wherein R¹⁰ and R¹¹ together form a substituted or unsubstituted 6 membered aryl ring.

15. The compound of claim 4 or any one of claims 1-4, wherein R³ is —OH.

16. The compound of claim 4 or any one of claims 1-4, wherein R¹⁰ and R¹¹ are each independently selected from the group of hydrogen, —OR¹², —SR¹², —N(R¹²)₂, —NO₂, —CN, —C(═O)OR¹², —C(═O)N(R¹²)₂, —C(═S)OR¹², —C(═S)SR¹², —C(═O)SR¹², C₁-C₁₆ substituted or unsubstituted alkyl, C₁-C₁₆ substituted or unsubstituted alkenyl, substituted or unsubstituted C₆ aryl, substituted or unsubstituted C₃-C₈ cycloalkyl, substituted or unsubstituted C₃-C₈ cycloalkenyl, substituted or unsubstituted C₃-C₈ cycloalkynyl, substituted or unsubstituted heterocycloalkyl, substituted or unsubstituted heterocycloalkenyl, substituted or unsubstituted heterocycloalkynyl, or C₁-C₁₆ substituted or unsubstituted alkynyl.

17. The compound of claim 14, wherein R³ is —OH or a halogen.

18. The compound of claim 14, wherein R⁹ is hydrogen.

19. The compound of claim 14, wherein R⁹ is hydrogen and R³ is —OH or a halogen.

20. The compound of claim 19, wherein R² is —C(═O)—N(R¹²)₂, or —C(═O)—OR¹².

21. The compound of claim 16, wherein R³ is —OH or a halogen.

22. The compound of claim 16, wherein R⁹ is hydrogen.

23. The compound of claim 16, wherein R⁹ is hydrogen and R³ is —OH or a halogen.

24. The compound of claim 22, wherein R² is —C(═O)—N(R¹²)₂, or —C(═O)—OR¹².

25. A compound selected from: 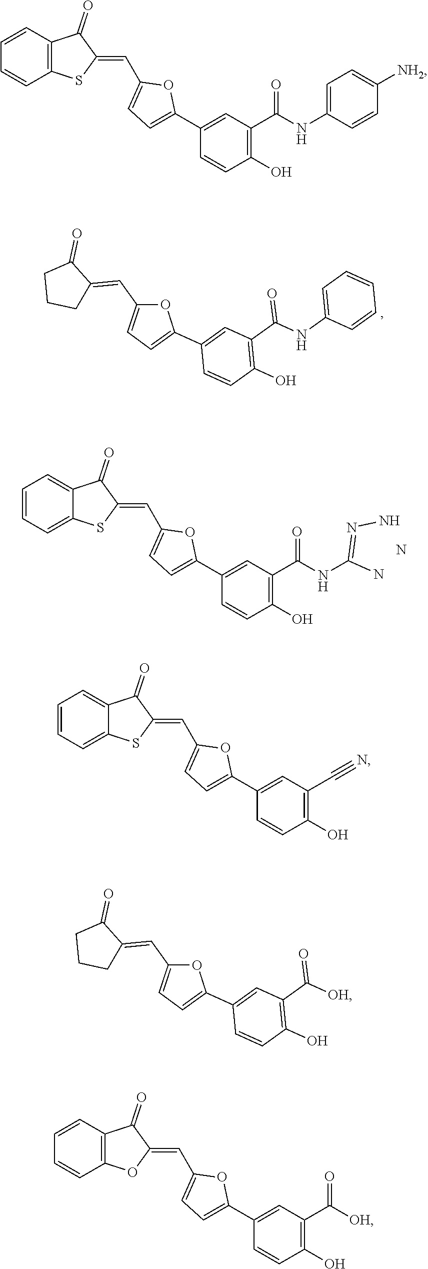 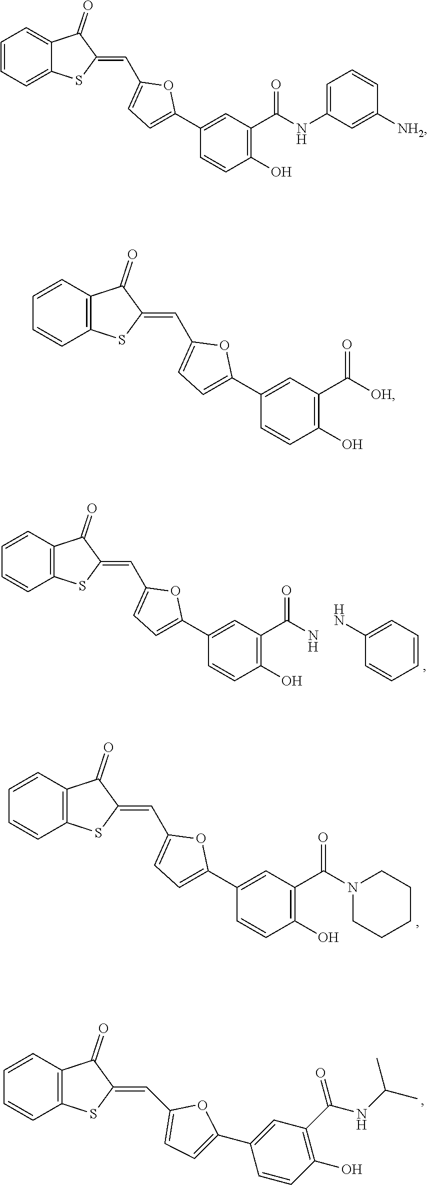 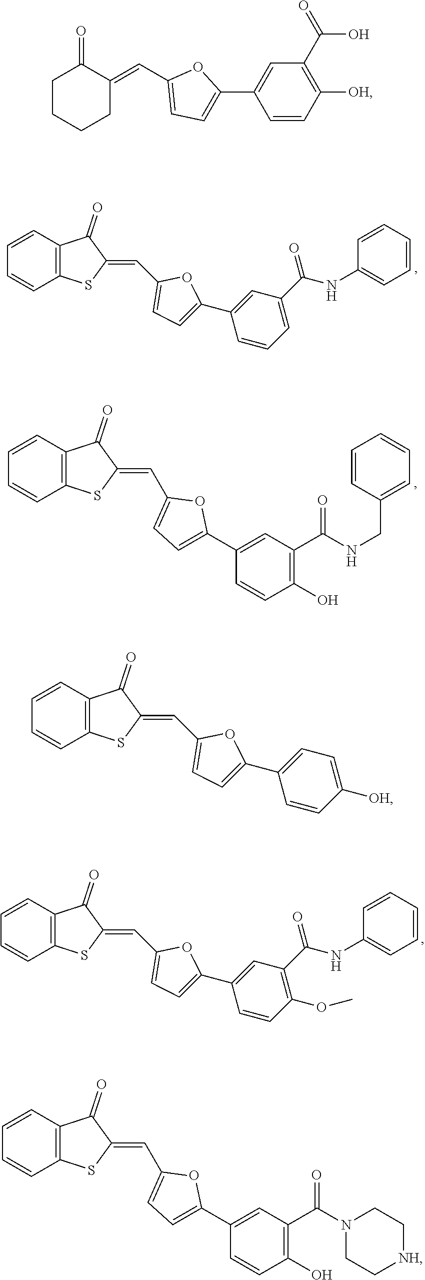 [SMILES: O=C1CCCC/C1=C\c1ccc(-c2ccc(O)c(C(=O)O)c2)o1 ; O=C(Nc1ccccc1)c1cccc(-c2ccc(/C=C3\Sc4ccccc4C3=O)o2)c1 ; O=C(NCc1ccccc1)c1cc(-c2ccc(/C=C3\Sc4ccccc4C3=O)o2)ccc1O ; O=C1/C(=C/c2ccc(-c3ccc(O)cc3)o2)Sc2ccccc21 ; COc1ccc(-c2ccc(/C=C3\Sc4ccccc4C3=O)o2)cc1C(=O)Nc1ccccc1 ; O=C1/C(=C/c2ccc(-c3ccc(O)c(C(=O)N4CCNCC4)c3)o2)Sc2ccccc21] 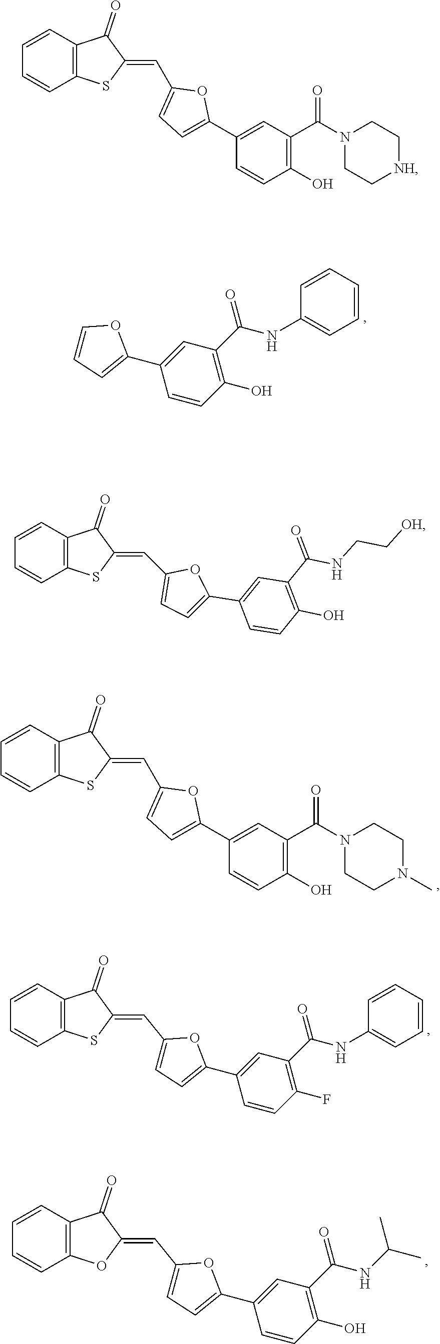 [SMILES: O=C1/C(=C/c2ccc(-c3ccc(O)c(C(=O)N4CCNCC4)c3)o2)Sc2ccccc21 ; O=C(Nc1ccccc1)c1cc(-c2ccco2)ccc1O ; O=C(NCCO)c1cc(-c2ccc(/C=C3\Sc4ccccc4C3=O)o2)ccc1O ; CN1CCN(C(=O)c2cc(-c3ccc(/C=C4\Sc5ccccc5C4=O)o3)ccc2O)CC1 ; O=C(Nc1ccccc1)c1cc(-c2ccc(/C=C3\Sc4ccccc4C3=O)o2)ccc1F ; CC(C)NC(=O)c1cc(-c2ccc(/C=C3\Oc4ccccc4C3=O)o2)ccc1O] 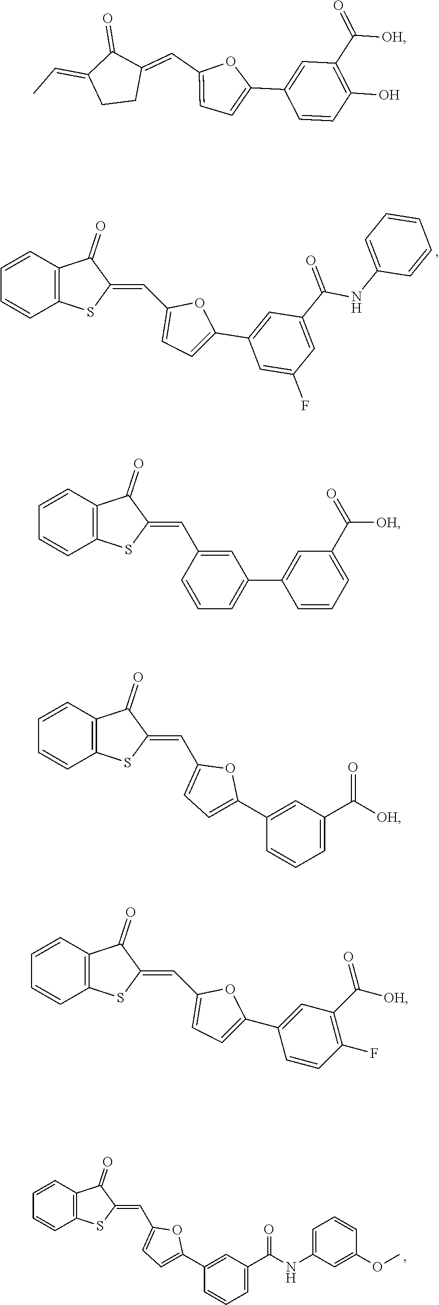 [SMILES: C/C=C1\CC/C(=C\c2ccc(-c3ccc(O)c(C(=O)O)c3)o2)C1=O ; O=C(Nc1ccccc1)c1cc(F)cc(-c2ccc(/C=C3\Sc4ccccc4C3=O)o2)c1 ; O=C(O)c1cccc(-c2cccc(/C=C3\Sc4ccccc4C3=O)c2)c1 ; O=C(O)c1cccc(-c2ccc(/C=C3\Sc4ccccc4C3=O)o2)c1 ; O=C(O)c1cc(-c2ccc(/C=C3\Sc4ccccc4C3=O)o2)ccc1F ; COc1cccc(NC(=O)c2cccc(-c3ccc(/C=C4\Sc5ccccc5C4=O)o3)c2)c1] 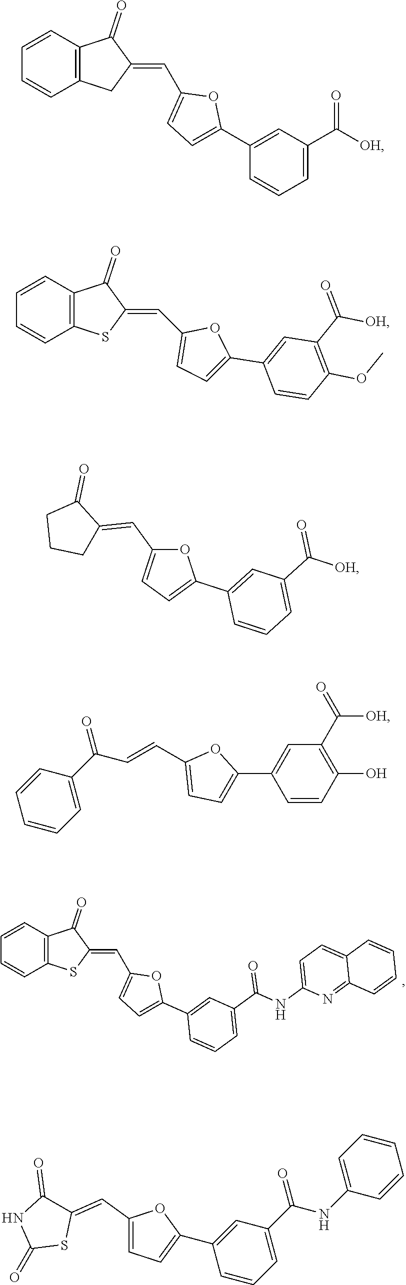 [SMILES: O=C(O)c1cccc(-c2ccc(/C=C3\Cc4ccccc4C3=O)o2)c1 ; COc1ccc(-c2ccc(/C=C3\Sc4ccccc4C3=O)o2)cc1C(=O)O ; O=C1CCC/C1=C\c1ccc(-c2cccc(C(=O)O)c2)o1 ; O=C(/C=C/c1ccc(-c2ccc(O)c(C(=O)O)c2)o1)c1ccccc1 ; O=C(Nc1ccc2ccccc2n1)c1cccc(-c2ccc(/C=C3\Sc4ccccc4C3=O)o2)c1 ; O=C1NC(=O)/C(=C/c2ccc(-c3cccc(C(=O)Nc4ccccc4)c3)o2)S1] 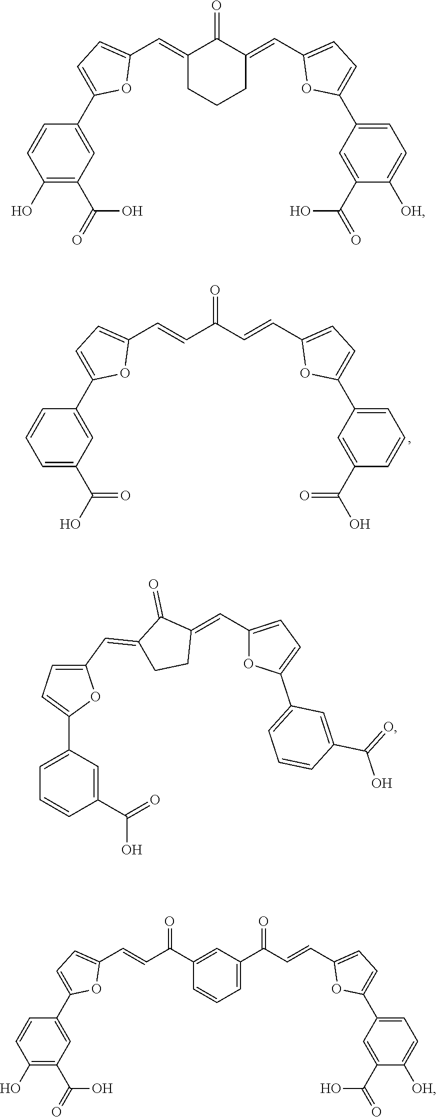 [SMILES: O=C1/C(=C/c2ccc(-c3ccc(O)c(C(=O)O)c3)o2)CCC/C1=C\c1ccc(-c2ccc(O)c(C(=O)O)c2)o1 ; O=C(/C=C/c1ccc(-c2cccc(C(=O)O)c2)o1)/C=C/c1ccc(-c2cccc(C(=O)O)c2)o1 ; O=C1/C(=C/c2ccc(-c3cccc(C(=O)O)c3)o2)CC/C1=C\c1ccc(-c2cccc(C(=O)O)c2)o1 ; O=C(/C=C/c1ccc(-c2ccc(O)c(C(=O)O)c2)o1)c1cccc(C(=O)/C=C/c2ccc(-c3ccc(O)c(C(=O)O)c3)o2)c1] 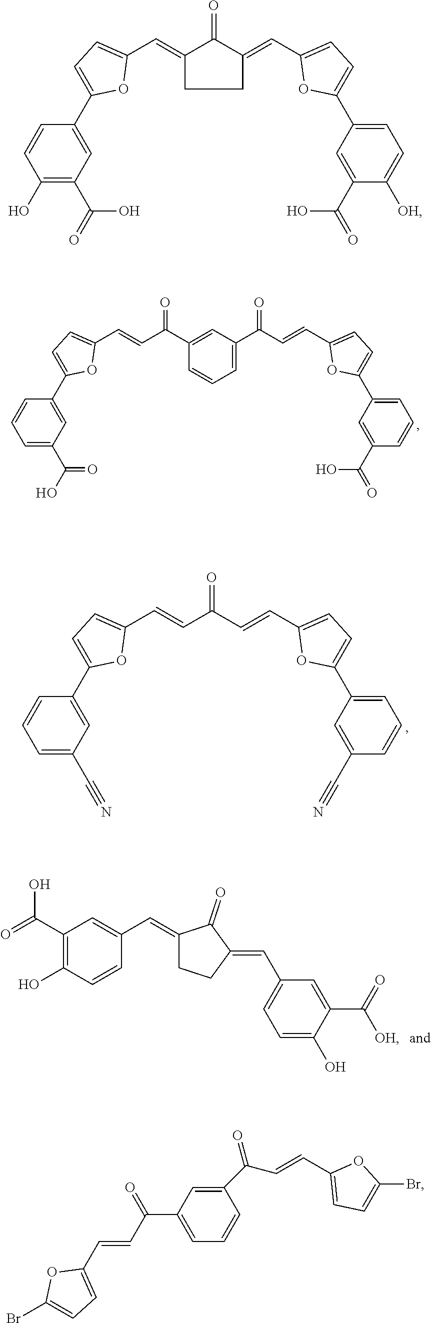 [SMILES: O=C1/C(=C/c2ccc(-c3ccc(O)c(C(=O)O)c3)o2)CC/C1=C\c1ccc(-c2ccc(O)c(C(=O)O)c2)o1 ; O=C(O)c1cccc(-c2ccc(/C=C/C(=O)c3cccc(C(=O)/C=C/c4ccc(-c5cccc(C(=O)O)c5)o4)c3)o2)c1 ; N#Cc1cccc(-c2ccc(/C=C/C(=O)/C=C/c3ccc(-c4cccc(C#N)c4)o3)o2)c1 ; O=C1/C(=C/c2ccc(O)c(C(=O)O)c2)CC/C1=C\c1ccc(O)c(C(=O)O)c1 ; "and" ; O=C(/C=C/c1ccc(Br)o1)c1cccc(C(=O)/C=C/c2ccc(Br)o2)c1]
or a pharmaceutically acceptable salt thereof.

26. A method of treating cancer comprising administering a therapeutically effective dose of an SHP2 inhibiting compound of Formula Ia: 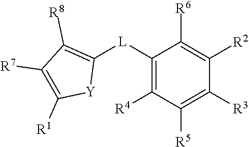
or a pharmaceutically acceptable salt thereof, wherein,
R¹ is: 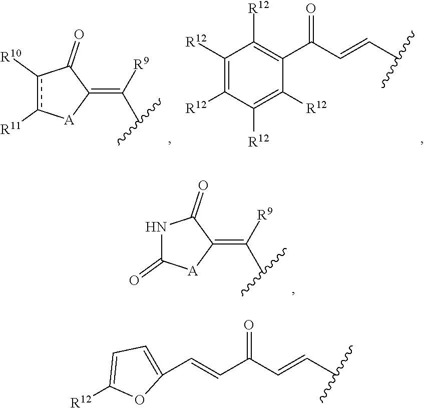
or hydrogen;
R² is selected from the group of: —H, halogen, —OH, —C═O—N(R¹³)₂, —CN, —C═O—OH, —C═O—NR¹³—N(R¹³)₂, —C═O—R¹², —C(═O)O—R¹³, —R¹², —O—R¹³, C₁-C₁₆ substituted or unsubstituted alkyl, C₁-C₁₆ substituted or unsubstituted alkenyl, substituted or unsubstituted C₆ aryl, substituted or unsubstituted C₃-C₈ cycloalkyl, substituted or unsubstituted C₃-C₈ cycloalkenyl, substituted or unsubstituted C₃-C₈ cycloalkynyl, substituted or unsubstituted heterocycloalkyl, substituted or unsubstituted heterocycloalkenyl, substituted or unsubstituted heterocycloalkynyl, C₁-C₁₆ substituted or unsubstituted alkynyl, 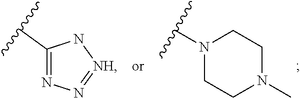
A is —CH₂—, —S—, —O—, —CH₂—CH₂—, —C(R¹²)₂—, or —C(R¹²)₂—C(R¹²)₂—;
Y is —O—, —S—, —NH— or —CH═CH—;
L is a bond, —CH═, or —C(═O)CH═CH—.
R³ is selected from the group of hydrogen, halogens, —C═O—N(R¹³)₂, —CN, —C═O—OH, —C═O—NR¹³—N(R¹³)₂, —C═O—R¹², —C(═O)O—R¹³, —R¹², —O—R¹³, —OH, —F, —Cl, —CF₃, —OCF₃, C₁-C₁₆ substituted or unsubstituted alkyl, C₁-C₁₆ substituted or unsubstituted alkenyl, substituted or unsubstituted C₆ aryl, substituted or unsubstituted C₃-C₈ cycloalkyl, substituted or unsubstituted C₃-C₈ cycloalkenyl, substituted or unsubstituted C₃-C₈ cycloalkynyl, substituted or unsubstituted heterocycloalkyl, substituted or unsubstituted heterocycloalkenyl, substituted or unsubstituted heterocycloalkynyl, or C₁-C₁₆ substituted or unsubstituted alkynyl;
R⁴, R⁵, R⁶, R⁷, R⁸, and R⁹ are each independently hydrogen, halogen, —OR¹³, —SR¹³, —N(R¹³)₂, —NO₂, —C(O)OR¹³, —C(O)N(R¹³)₂, —C(S)OR¹³, —C(S)SR¹³, —C(O)SR¹³, C₁-C₁₆ substituted or unsubstituted alkyl, C₁-C₁₆ substituted or unsubstituted alkenyl, substituted or unsubstituted C₆ aryl, substituted or unsubstituted C₃-C₈ cycloalkyl, substituted or unsubstituted C₃-C₈ cycloalkenyl, substituted or unsubstituted C₃-C₈ cycloalkynyl, substituted or unsubstituted heterocycloalkyl, substituted or unsubstituted heterocycloalkenyl, substituted or unsubstituted heterocycloalkynyl, or C₁-C₁₆ substituted or unsubstituted alkynyl;
R¹⁰ and R¹¹ either together form a substituted or unsubstituted 6 membered aryl ring, or are each independently hydrogen, —OR¹³, —SR¹³, —N(R¹³)₂, —NO₂, —CN, —C(O)OR¹³, —C(O)N(R¹³)₂, —C(S)OR¹³, —C(S)SR¹³, —C(O)SR¹³, C₁-C₁₆ substituted or unsubstituted alkyl, C₁-C₁₆ substituted or unsubstituted alkenyl, substituted or unsubstituted C₆ aryl, substituted or unsubstituted C₃-C₈ cycloalkyl, substituted or unsubstituted C₃-C₈ cycloalkenyl, substituted or unsubstituted C₃-C₈ cycloalkynyl, substituted or unsubstituted heterocycloalkyl, substituted or unsubstituted heterocycloalkenyl, substituted or unsubstituted heterocycloalkynyl, or C₁-C₁₆ substituted or unsubstituted alkynyl;
R¹² are each independently hydrogen, halogens, —OH, —CN, —SH, —NO₂, 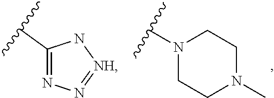
C₁-C₁₆ substituted or unsubstituted alkyl, C₁-C₁₆ substituted or unsubstituted alkyl, C₁-C₁₆ substituted or unsubstituted alkenyl, substituted or unsubstituted C₆ aryl, substituted or unsubstituted C₃-C₈ cycloalkyl, substituted or unsubstituted C₃-C₈ cycloalkenyl, substituted or unsubstituted C₃-C₈ cycloalkynyl, substituted or unsubstituted heterocycloalkyl, substituted or unsubstituted heterocycloalkenyl, substituted or unsubstituted heterocycloalkynyl, or C₁-C₁₆ substituted or unsubstituted alkynyl;
R¹³ are each independently C₁-C₁₆ substituted or unsubstituted alkyl, C₁-C₁₆ substituted or unsubstituted alkyl, C₁-C₁₆ substituted or unsubstituted alkenyl, substituted or unsubstituted C₆ aryl, substituted or unsubstituted C₃-C₈ cycloalkyl, substituted or unsubstituted C₃-C₈ cycloalkenyl, substituted or unsubstituted C₃-C₈ cycloalkynyl, substituted or unsubstituted heterocycloalkyl, substituted or unsubstituted heterocycloalkenyl, substituted or unsubstituted heterocycloalkynyl, or C₁-C₁₆ substituted or unsubstituted alkynyl;
with the proviso that the compound is not:

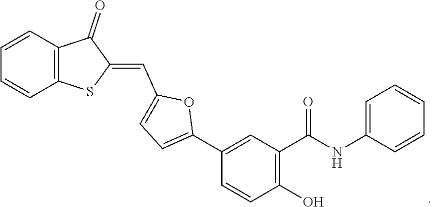 [SMILES: O=C(Nc1ccccc1)c1cc(-c2ccc(/C=C3\Sc4ccccc4C3=O)o2)ccc1O]

27. The method of claim 26, wherein R¹ is

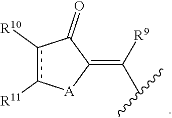

28. The method of claim 27, wherein R⁹ is hydrogen.

29. The method of claim 27, wherein A is —O—, —S—, —CH₂—, or —CH₂CH₂—.

30. The method of claim 26, wherein R¹ is

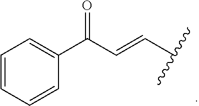 [SMILES: */C=C/C(=O)c1ccccc1]

31. The method of claim 30, wherein R³ is —OH or a halogen.

32. The method of claim 30, wherein R² is —C(═O)—N(R¹²)₂, or —C(═O)—OR¹².

33. The method of claim 26, wherein R¹ is H.

34. The method of claim 26, wherein R³ is —OH or a halogen.

35. The method of claim 26, wherein R² is —C(═O)N(R¹²)₂.

36. The method of claim 31, wherein R³ is —OH.

37. The method of claim 31, wherein R² is —C═O—NHR¹².

38. The method of claim 37, wherein R² is

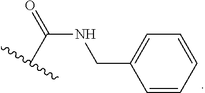 [SMILES: *C(=O)NCc1ccccc1]

39. The method of claim 29, wherein R¹⁰ and R¹¹ together form a substituted or unsubstituted 6 membered aryl ring.

40. The method of claim 29, wherein R³ is —OH.

41. The method of claim 29, wherein R¹⁰ and R¹¹ are each independently selected from the group of hydrogen, —OR¹², —SR¹², —N(R¹²)₂, —NO₂, —CN, —C(═O)OR¹², —C(═O)N(R¹²)₂, —C(═S)OR¹², —C(═S)SR¹², —C(═O)SR¹², C₁-C₁₆ substituted or unsubstituted alkyl, C₁-C₁₆ substituted or unsubstituted alkenyl, substituted or unsubstituted C₆ aryl, substituted or unsubstituted C₃-C₈ cycloalkyl, substituted or unsubstituted C₃-C₈ cycloalkenyl, substituted or unsubstituted C₃-C₈ cycloalkynyl, substituted or unsubstituted heterocycloalkyl, substituted or unsubstituted heterocycloalkenyl, substituted or unsubstituted heterocycloalkynyl, or C₁-C₁₆ substituted or unsubstituted alkynyl.

42. The method of claim 39, wherein R³ is —OH or a halogen.

43. The method of claim 39, wherein R⁹ is hydrogen.

44. The method of claim 39, wherein R⁹ is hydrogen and R³ is —OH or a halogen.

45. The method of claim 44, wherein R² is —C(═O)—N(R¹²)₂, or —C(═O)—OR¹².

46. The method of claim 41, wherein R³ is —OH or a halogen.

47. The method of claim 41, wherein R⁹ is hydrogen.

48. The method of claim 41, wherein R⁹ is hydrogen and R³ is —OH or a halogen.

49. The method of claim 47, wherein R² is —C(═O)—N(R¹²)₂, or —C(═O)—OR¹².

50. A method of treating cancer comprising administering a therapeutically effective dose of a compound selected from: 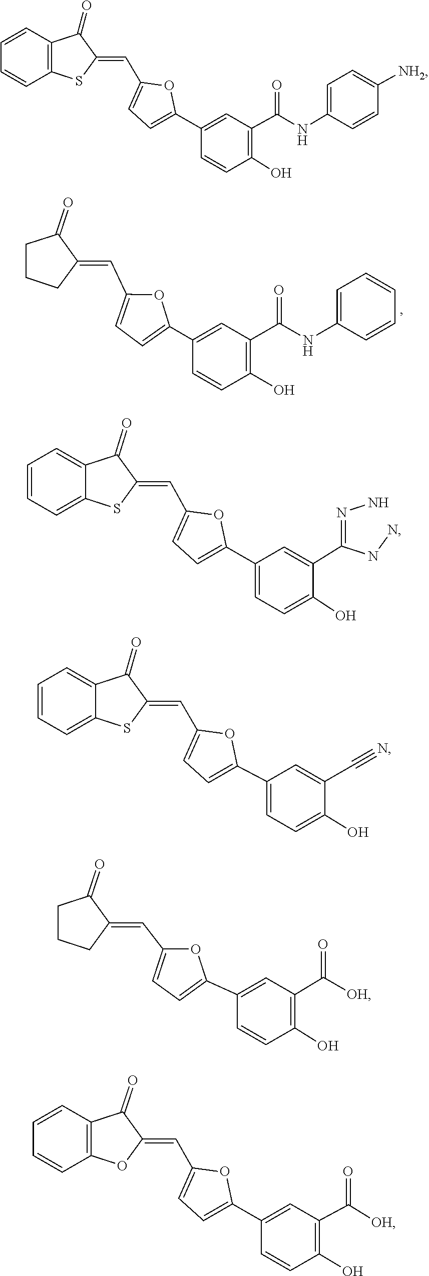 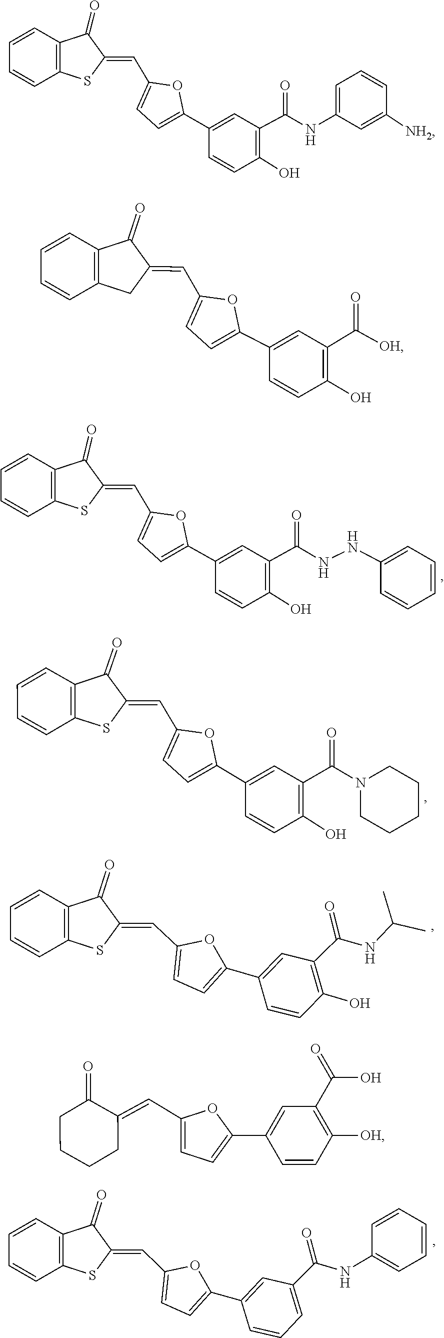 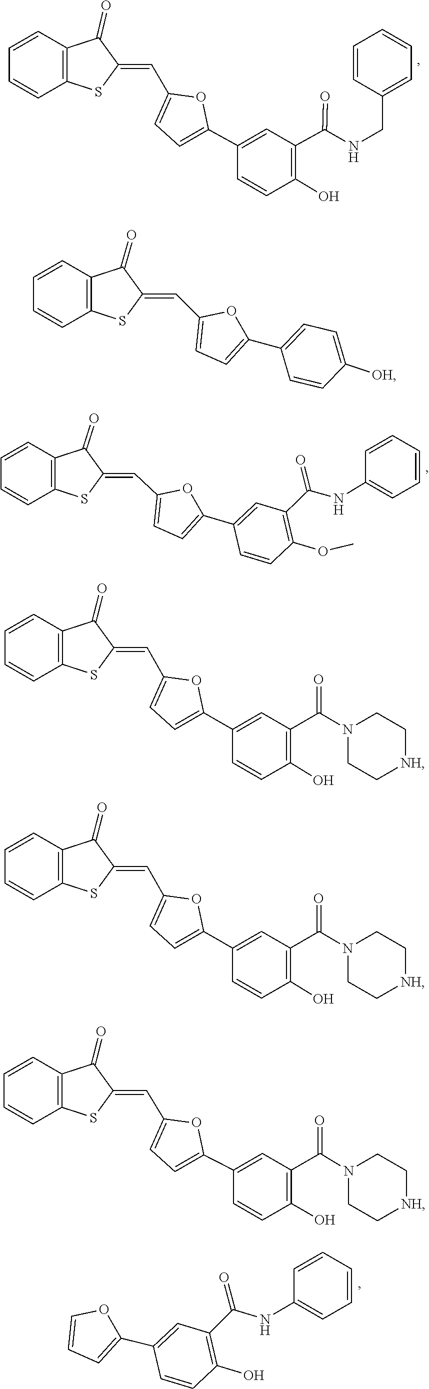 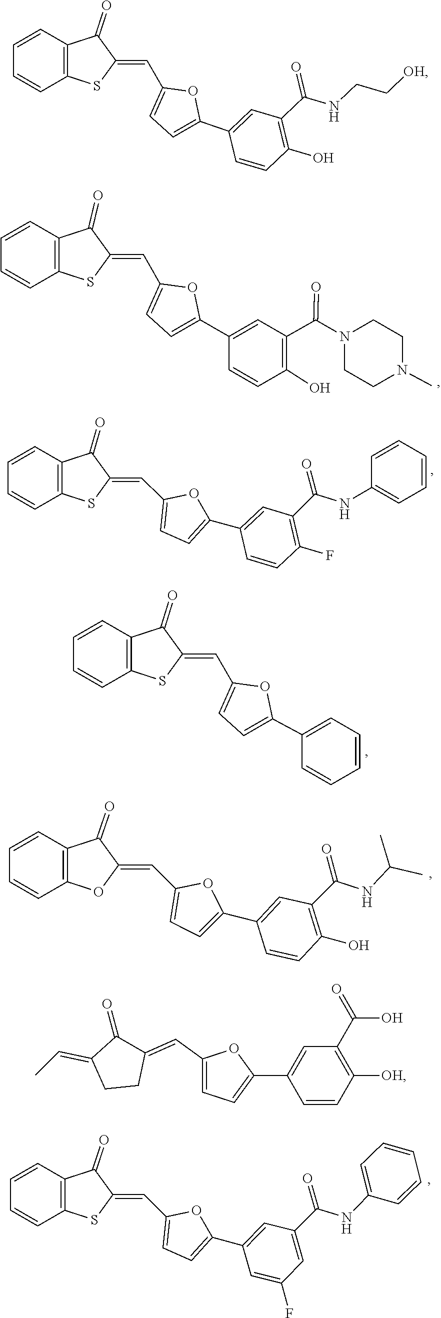 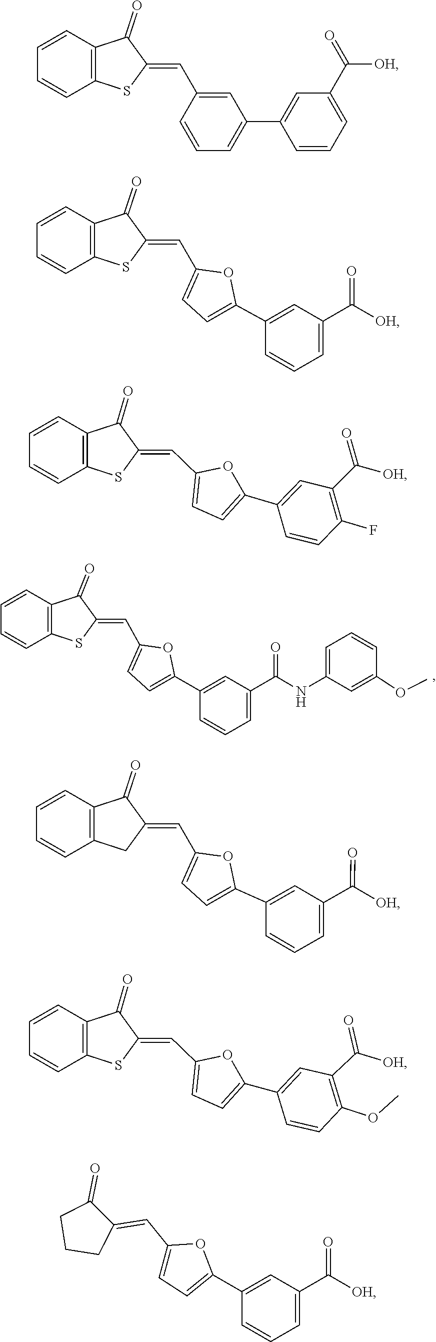 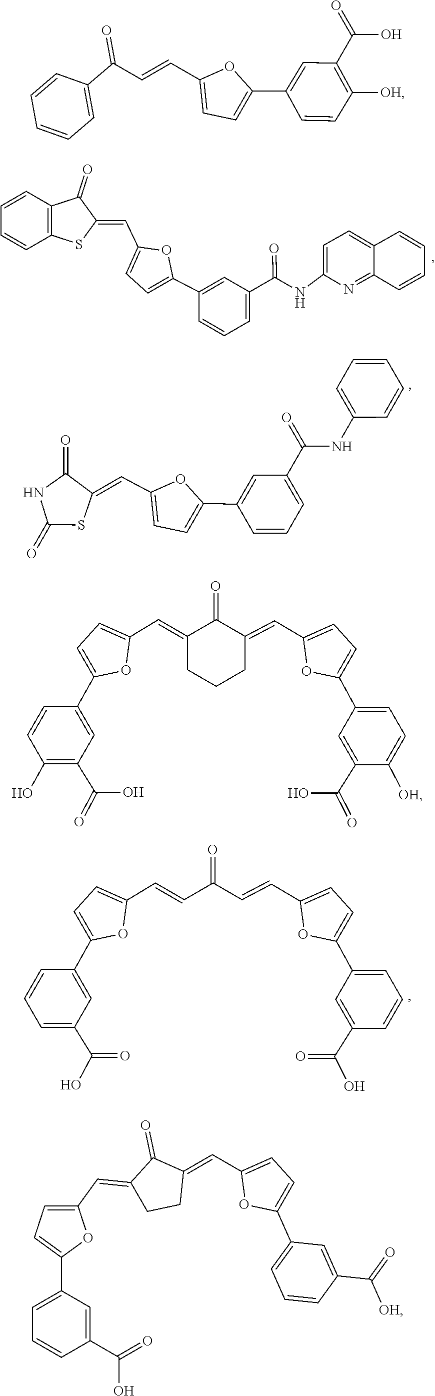 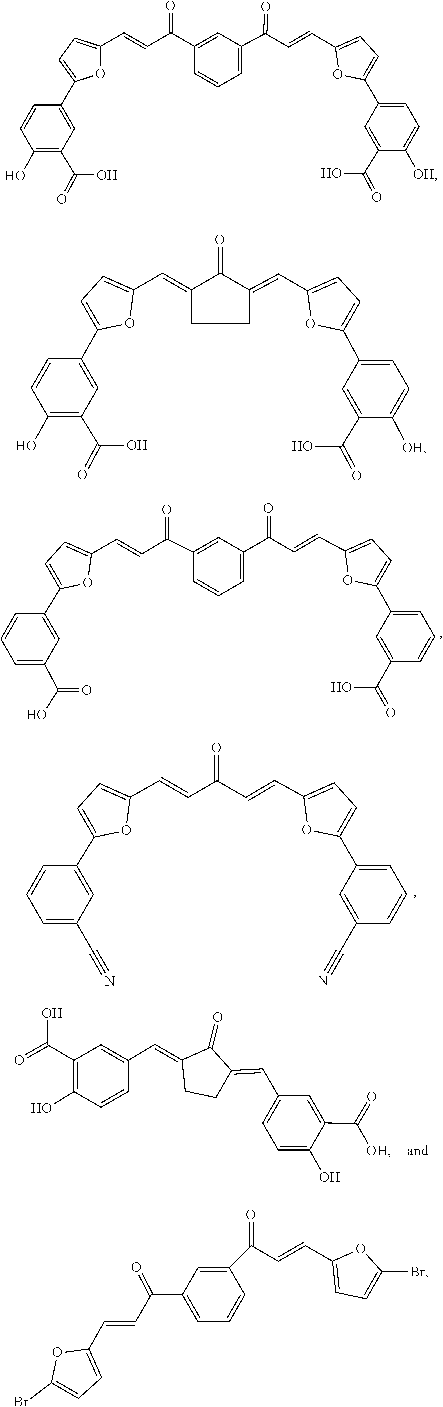 [SMILES: O=C(/C=C/c1ccc(-c2ccc(O)c(C(=O)O)c2)o1)c1cccc(C(=O)/C=C/c2ccc(-c3ccc(O)c(C(=O)O)c3)o2)c1 ; O=C1/C(=C/c2ccc(-c3ccc(O)c(C(=O)O)c3)o2)CC/C1=C\c1ccc(-c2ccc(O)c(C(=O)O)c2)o1 ; O=C(O)c1cccc(-c2ccc(/C=C/C(=O)c3cccc(C(=O)/C=C/c4ccc(-c5cccc(C(=O)O)c5)o4)c3)o2)c1 ; N#Cc1cccc(-c2ccc(/C=C/C(=O)/C=C/c3ccc(-c4cccc(C#N)c4)o3)o2)c1 ; O=C1/C(=C/c2ccc(O)c(C(=O)O)c2)CC/C1=C\c1ccc(O)c(C(=O)O)c1 ; O=C(/C=C/c1ccc(Br)o1)c1cccc(C(=O)/C=C/c2ccc(Br)o2)c1]
or a pharmaceutically acceptable salt thereof.

51. The compound of claim 1, wherein R¹ is —CH═Z;
Z is 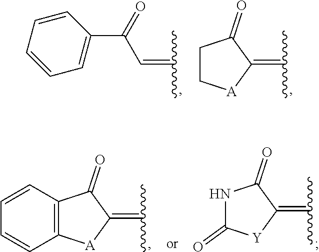
A is —CH₂—, —S—, —O—, or —CH₂—CH₂—;
Y is —O— or —S—,
R² is —CN, —C(O)N(R¹²)₂, —C(O)N(R¹²)N(R¹²)₂, —C(O)R¹² or 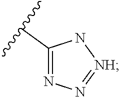
R⁴, R⁵, and R⁶ are each independently selected from hydrogen, halogen, and C₁₋₆ alkyl;
R⁷ and R⁸ are each independently selected from hydrogen, halogen, C₁₋₆ alkyl, —OR¹³, and —SR¹³;
R¹² are each independently selected from hydrogen, C₁₋₆ alkyl, C₆-C₁₀ aryl, C₃₋₆ cycloalkyl, 3-10 membered heterocycloalkyl, wherein each alkyl, aryl, cycloalkyl, or heterocycloalkyl is optionally substituted with 1-5 substituents independently selected from halogen, hydroxyl, amino, C₁₋₆ alkyl, C₁₋₆ alkoxyl, and phenyl; and
R¹³ are each independently C₁-C₆ alkyl.

52. The compound of claim 51, wherein the compound is selected from: 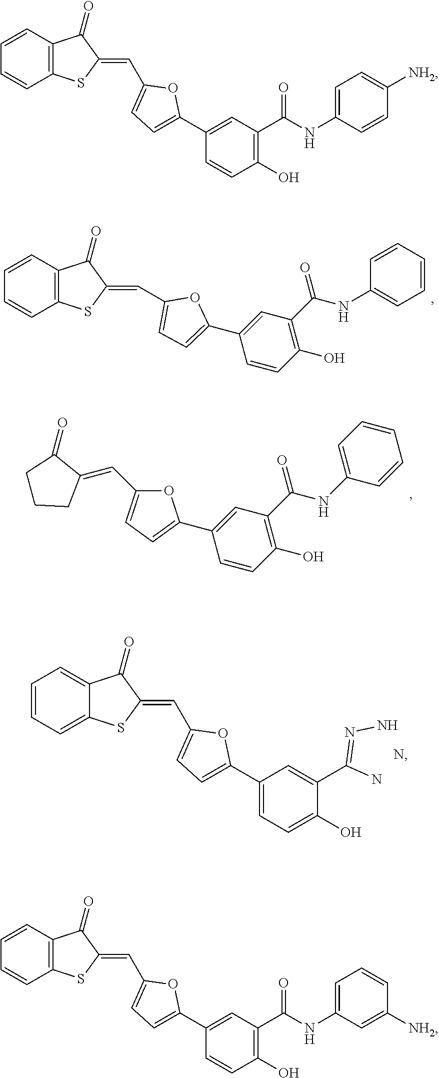 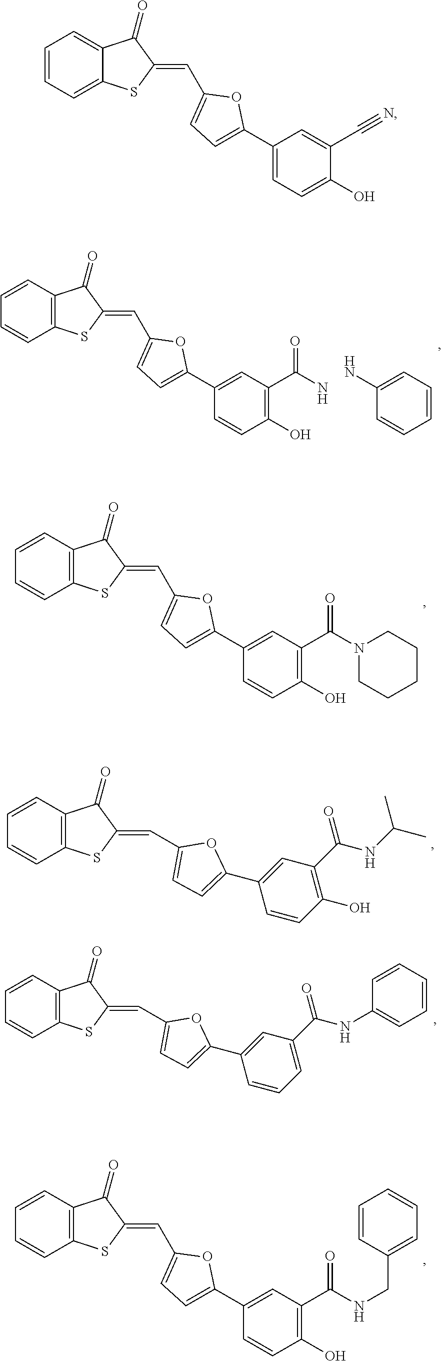 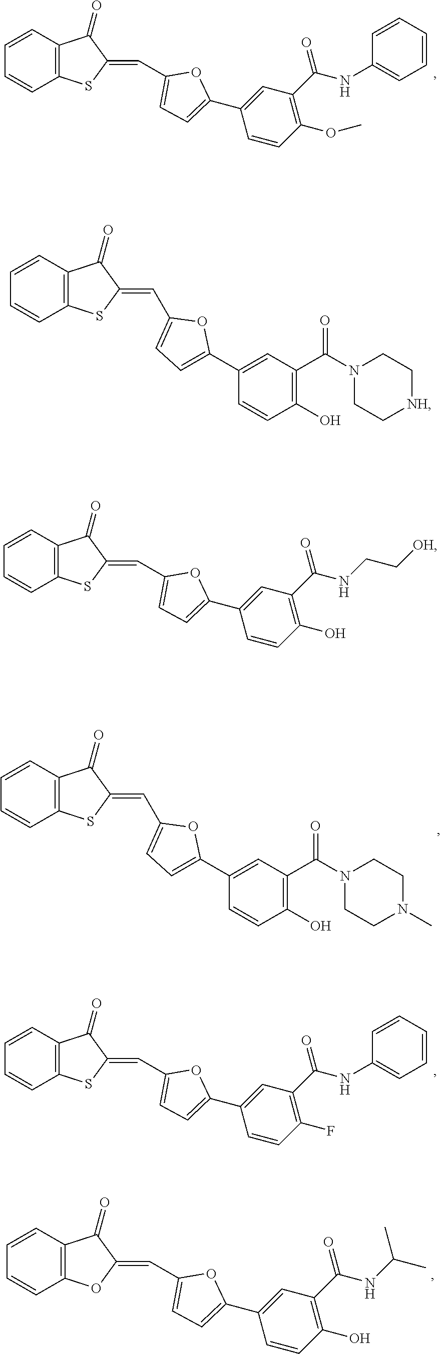 [SMILES: COc1ccc(-c2ccc(/C=C3\Sc4ccccc4C3=O)o2)cc1C(=O)Nc1ccccc1 ; O=C1/C(=C/c2ccc(-c3ccc(O)c(C(=O)N4CCNCC4)c3)o2)Sc2ccccc21 ; O=C(NCCO)c1cc(-c2ccc(/C=C3\Sc4ccccc4C3=O)o2)ccc1O ; CN1CCN(C(=O)c2cc(-c3ccc(/C=C4\Sc5ccccc5C4=O)o3)ccc2O)CC1 ; O=C(Nc1ccccc1)c1cc(-c2ccc(/C=C3\Sc4ccccc4C3=O)o2)ccc1F ; CC(C)NC(=O)c1cc(-c2ccc(/C=C3\Oc4ccccc4C3=O)o2)ccc1O] 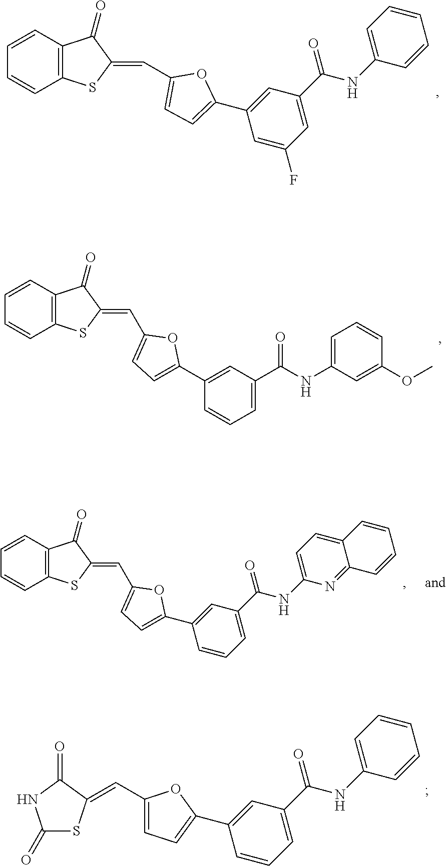 [SMILES: O=C(Nc1ccccc1)c1cc(F)cc(-c2ccc(/C=C3\Sc4ccccc4C3=O)o2)c1 ; COc1cccc(NC(=O)c2cccc(-c3ccc(/C=C4\Sc5ccccc5C4=O)o3)c2)c1 ; O=C(Nc1ccc2ccccc2n1)c1cccc(-c2ccc(/C=C3\Sc4ccccc4C3=O)o2)c1 ; O=C1NC(=O)/C(=C/c2ccc(-c3cccc(C(=O)Nc4ccccc4)c3)o2)S1]
or a pharmaceutically acceptable salt thereof.

53. A compound selected from: 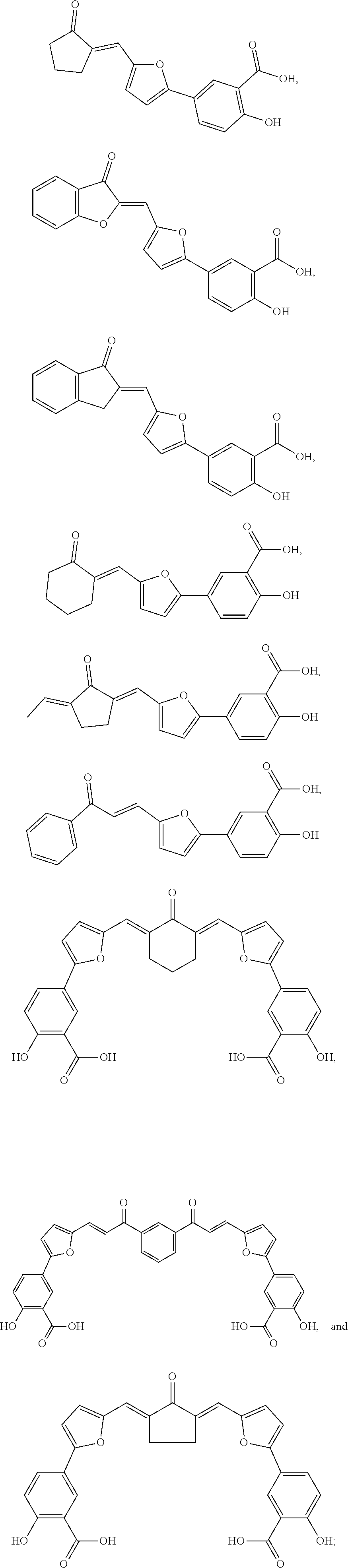
or a pharmaceutically acceptable salt thereof.

54. A compound selected from: 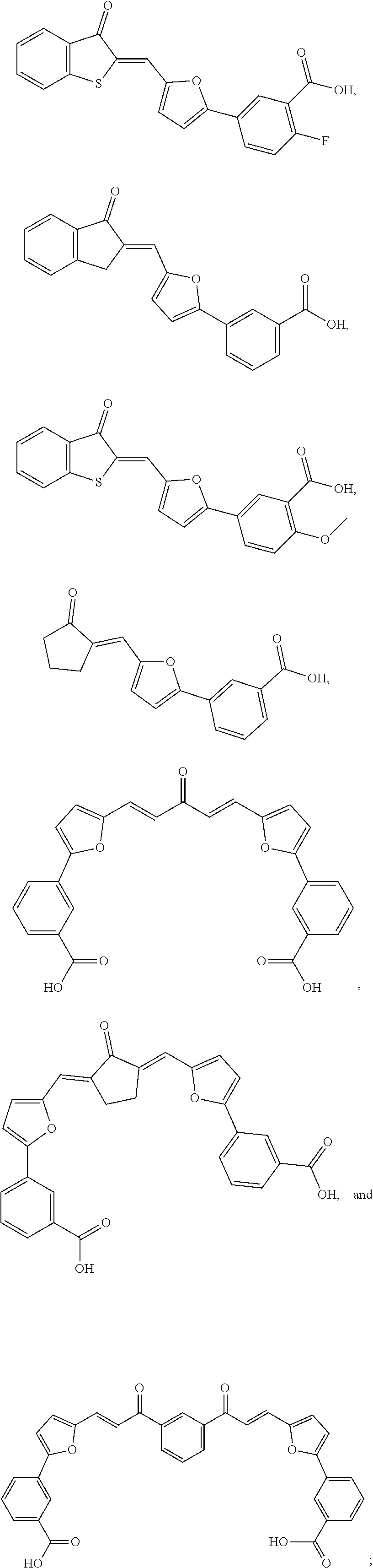
or a pharmaceutically acceptable salt thereof.

55. A compound, selected from: 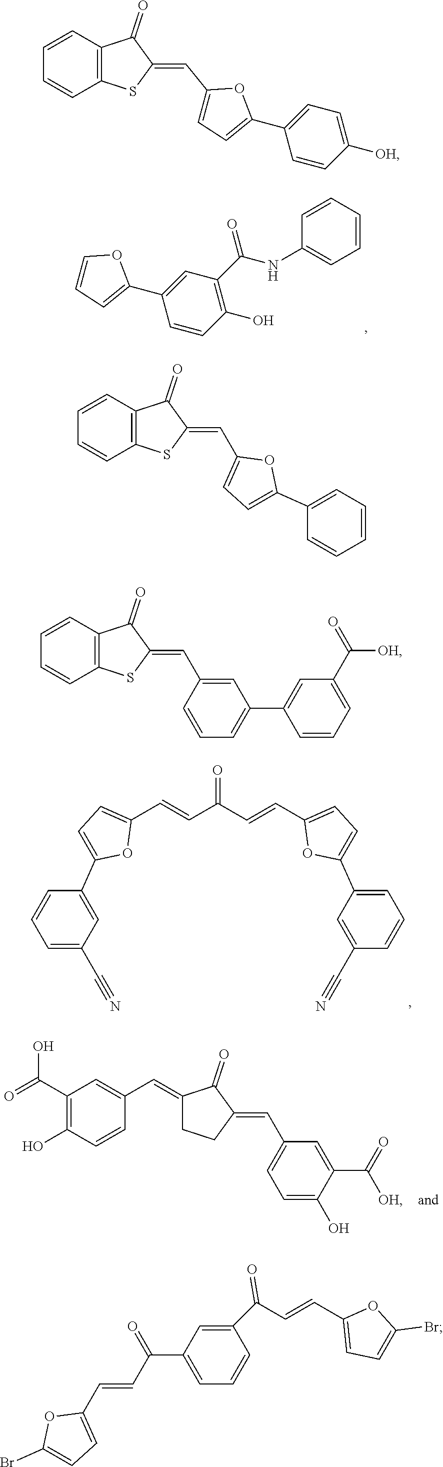
or a pharmaceutically acceptable salt thereof.

56. A pharmaceutical composition comprising a compound of any one of claims 1-25 and 51-55, or a pharmaceutically acceptable salt thereof, and a pharmaceutically acceptable excipient.

57. A crystalline form of a compound of any one of claims 1-25 and 51-55 or a pharmaceutically acceptable salt thereof, or a solvate or hydrate thereof.

58. An amorphous form of a compound of any one of claims 1-25 and 51-55 or a pharmaceutically acceptable salt thereof, or a solvate or hydrate thereof.

59. A solid form of a compound of any one of claims 1-25 and 51-55 or a pharmaceutically acceptable salt thereof, or a solvate or hydrate thereof.

60. A method of treating cancer comprising administering a therapeutically effective dose of an SHP2 inhibiting compound to a patient in need thereof, wherein the SHP2 inhibiting compound is a compound of any one of claims 51-55 or a pharmaceutically acceptable salt thereof.

61. The method of claim 60, wherein the cancer comprises cells expressing SHP2 that comprises a E76K mutation.

62. The method of claim 60 wherein the SHP2 inhibiting compound inhibits wild type SHP2.

63. The method of any one of claims 60-62 wherein the SHP2 inhibiting compound inhibits SHP2 oncogenic variant E76K.

64. Use of a compound of any one of claims 51-55 as a treatment for cancer.

65. The use of claim 64 wherein the cancer comprises cells expressing SHP2 oncogenic variant E76K.

66. Use of a compound of any one of claims 51-55 in the manufacture of a medicament for the treatment of cancer.

67. The use of claim 66 wherein the cancer comprises cells expressing SHP2 oncogenic variant E76K.
